(19) United States
(12) Patent Application Publication
(10) Pub. No.: US 2023/0270845 A1
(43) Pub. Date: Aug. 31, 2023

(54) TREATMENT AND PREVENTION OF CONDITIONS ASSOCIATED WITH RESPIRATORY DISEASES

(71) Applicant: UNIVERSITY OF HOUSTON SYSTEM, Houston, TX (US)

(72) Inventors: Navin Varadarajan, Houston, TX (US); Xinli Liu, Houston, TX (US)

(73) Assignee: UNIVERSITY OF HOUSTON SYSTEM, Houston, TX (US)

(21) Appl. No.: 18/017,659

(22) PCT Filed: Jul. 22, 2021
(86) PCT No.: PCT/US2021/042684

§ 371 Date: Jan. 23, 2023

Related U.S. Application Data
(60) Provisional application No. 63/055,165, filed on Jul. 22, 2020.
(60) Provisional application No. 63/060,503, filed on Aug. 3, 2020.
(60) Provisional application No. 63/108,980, filed on Nov. 3, 2020.

(51) Int. Cl. A61K 39/215; A61K 39/39; A61P 31/14

(52) U.S. Cl. CPC A61K 39/215; A61K 39/39; A61P 31/14; A61K 2039/55555

(57) ABSTRACT

Described herein are compositions and methods for the prevention of pathogens such as conditions in an animal (e.g., a subject). In some cases, a composition or method described herein can comprise a liposome, which may be used to encapsulate one or more STING agonists. In some cases, a liposome of a composition or method described herein may comprise one or more antigens attached to, integrated into, or associated with a liposomal membrane of the liposome.

### CROSS-REFERENCE TO RELATED APPLICATIONS

The present application claims the benefit of U.S. Provisional Patent Application No. 63/055,165, filed on Jul. 22, 2020; U.S. Provisional Patent Application No. 63/060,503, filed on Aug. 3, 2020; and U.S. Provisional Patent Application No. 63/108,980, filed on Nov. 3, 2020. The entirety of each of the aforementioned applications is incorporated herein by reference.

### STATEMENT REGARDING FEDERALLY SPONSORED RESEARCH

This invention was made with government support under U01AI148118, awarded by the National Institutes of Health. The government has certain rights in the invention.

### BACKGROUND

Safe and durable preventative and therapeutic compositions are urgently needed to address various diseases, such as infections caused by pathogens and various types of cancer. Numerous embodiments of the present disclosure address the aforementioned need.

### SUMMARY

In various aspects, a composition for use in preventing a disease in an animal comprises: a lipid-based particle comprising an antigen and a modulator, wherein the modulator is a pattern recognition receptor agonist. In various aspects, a composition for use in treating a disease in a subject comprises: a lipid-based particle comprising an antigen and a modulator, wherein the modulator is a pattern recognition receptor agonist. In some cases, the lipid-based particle is a liposome. In some cases, the lipid-based particle comprises DPPG (1,2-dipalmitoyl-sn-glycero-3-phospho-(1′-rac-glycerol)), DPPE (1,2-bis(diphenylphosphino)ethane), DPPC (1,2-dipalmitoyl-sn-glycero-3-phosphocholine), DPPE-PEG2000 (1,2-dipalmitoyl-sn-glycero-3-phosphoethanolamine-N-[methoxy(polyethylene glycol)-2000]), cholesterol, or a combination thereof. In some cases, the lipid-based particle comprises DPPC, DPPG, cholesterol, and DPPE-PEG2000. In some cases, the lipid-based particle comprises the DPPC, the DPPG, the cholesterol, and the DPPE-PEG2000 in a 10:1:1:1 ratio, respectively. In some cases, the antigen is associated with an outer surface of the lipid-based particle. In some cases, the antigen is covalently or non-covalently bound to the outer surface of the lipid-based particle. In some cases, at least a portion of the antigen is integrated into a membrane of the lipid-based particle. In some cases, at least a portion of the antigen is encapsulated within the lipid-based particle. In some cases, the antigen elicits an immune response against the disease. In some cases, the antigen comprises an attenuated pathogen or a portion thereof. In some cases, the antigen comprises a killed pathogen or a portion thereof. In some cases, the antigen comprises a peptide or a protein of a pathogen. In some cases, the antigen comprises a spike protein or portion thereof. In some cases, the antigen comprises a nucleocapsid protein or portion thereof. In some cases, the antigen comprises a chimeric protein. In some cases, the antigen is trimeric. In some cases, the antigen comprises an attenuated or killed version of a tumor cell. In some cases, the tumor cell is associated with a cancer. In some cases, the antigen comprises a peptide or a protein associated with a cancer cell. In some cases, the modulator is capable of activating or inhibiting of the stimulator of interferon genes (STING) pathway in the subject. In some cases, the modulator is an agonist of the STING pathway. In some cases, the modulator is selected from the group consisting of bis-(3′,5′)-cyclic dimeric guanosine monophosphate (c-di-GMP), cyclic guanosine monophosphate-adenosine monophosphate (cGAMP), amidobenzimidazole, derivatives of amidobenzimidazole, nucleotide modulators, plasmid DNA modulators or a combination thereof. In some cases, the modulator is an antagonist of the STING pathway. In some cases, the disease comprises an infection caused by a pathogen. In some cases, the pathogen is a respiratory pathogen. In some cases, the respiratory pathogen is a virus. In some cases, the virus is selected from an influenza virus, a parainfluenza virus, an adenovirus, an enterovirus, a coronavirus, a respiratory syncytial virus, a rhinovirus, a DNA virus, an RNA virus, or a combination thereof. In some cases, the influenza virus is an influenza A virus or an influenza B virus. In some cases, the virus is a respiratory syncytial virus. In some cases, the virus is a coronavirus. In some cases, the coronavirus is selected from a severe acute respiratory syndrome coronavirus (SARS-CoV), a severe acute respiratory syndrome-related coronavirus (SARSr-CoV), a human coronavirus 229E (HCoV-229E), a human coronavirus NL63 (HCoV-NL63), a human coronavirus OC43 (HCoV-OC43), a human coronavirus HKU1 (HCoV-HKU1), a Middle East respiratory syndrome-related coronavirus (MERS-CoV), a severe acute respiratory syndrome-related coronavirus 2 (SARS-CoV-2), a variant of SARS-CoV-2, or combinations thereof. In some cases, the disease is a respiratory disease. In some cases, the disease comprises a cancer. In some cases, the cancer is selected from tracheal cancer, lung cancer, bronchial cancer, epithelial cancer, blood cancer, breast cancer, melanoma, ovarian cancer, gynecological cancer, leukemias, lymphomas, prostate cancer, bladder cancer, colon cancer, gliomas, sarcomas, glioblastoma, or a combination thereof. In some cases, the cancer is lung cancer. In some cases, the composition is in the form of a vaccine. In some cases, the composition is suitable for an administration mode selected from the group consisting of intravenous administration, intramuscular administration, intradermal administration, intraperitoneal administration, subcutaneous administration, spray-based administration, aerosol-based administration, in ovo administration, oral administration, intraocular administration, intratracheal administration, intranasal administration, inhalational administration, or combinations thereof. In some cases, the composition is lyophilized. In some cases, the composition is formulated in unit dose formulation as a monotherapy. In some cases, the composition comprises an adjuvant. In some cases, the composition is suitable for developing immunity in the subject against the disease. In some cases, the composition elicits an immune response against the disease. In some cases, the composition elicits an innate immune response in the subject against the disease. In some cases, the innate immune response leads to the activation of interferon regulator factors (IRFs). In some cases, the innate immune response leads to the activation of nuclear factor κB (NF-κB). In some cases, the innate immune response leads to the synthesis and secretion of type-I and type-III interferons and the subsequent upregulation of IFN-stimulated genes (ISGs).

In various aspects, a method of preventing a disease in a subject comprises: administering to the subject a composition described above or herein. In various aspects, a method of treating a disease in a subject comprises: administering to the subject a composition described above or herein. In some cases, the composition is suitable for developing immunity in the subject against the disease. In some cases, administering the composition to the subject elicits an immune response in the subject against the disease. In some cases, administering the composition elicits an innate immune response in the subject against the disease. In some cases, the innate immune response leads to the activation of interferon regulator factors (IRFs) in the subject. In some cases, the innate immune response leads to the activation of nuclear factor κB (NF-κB). In some cases, the innate immune response leads to the synthesis and secretion of type-I and type-III interferons and the subsequent upregulation of IFN-stimulated genes (ISGs). In some cases, the innate immune response leads to associated adaptive immunity. In some cases, the disease is caused by a pathogen. In some cases, the pathogen is a respiratory pathogen. In some cases, the respiratory pathogen is a virus. In some cases, the virus is selected from a severe acute respiratory syndrome coronavirus (SARS-CoV), a severe acute respiratory syndrome-related coronavirus (SARSr-CoV), a human coronavirus 229E (HCoV-229E), a human coronavirus NL63 (HCoV-NL63), a human coronavirus OC43 (HCoV-OC43), a human coronavirus HKU1 (HCoV-HKU1), a Middle East respiratory syndrome-related coronavirus (MERS-CoV), a severe acute respiratory syndrome-related coronavirus 2 (SARS-CoV-2), a variant of SARS-CoV-2, or combinations thereof. In some cases, the disease is a cancer. In some cases, the cancer is selected from tracheal cancer, lung cancer, bronchial cancer, epithelial cancer, blood cancer, breast cancer, melanoma, ovarian cancer, gynecological cancer, a leukemia, a lymphoma, prostate cancer, bladder cancer, colon cancer, a glioma, a sarcoma, glioblastoma, or a combination thereof. In some cases, the cancer is lung cancer. In some cases, the composition is administered to the subject by intravenous administration, intramuscular administration, intradermal administration, intraperitoneal administration, subcutaneous administration, spray-based administration, aerosol-based administration, in ovo administration, oral administration, intraocular administration, intratracheal administration, intranasal administration, inhalational administration, or combinations thereof. In some cases, the composition is administered to the subject by intranasal administration. In some cases, the composition is administered to the subject by inhalation administration. In some cases, the composition is administered to the subject in single dose. In some cases, the composition is administered to the subject in at least two doses, a first dose and a second dose of the at least two doses being administered at an interval of at least 6 hours, at least 12 hours, at least 24 hours, at least 36 hours, at least 2 days, at least 3 days, at least 4 days, at least 5 days, at least 6 days, at least 7 days, at least 14 days, or at least 28 days. In some cases, the composition is administered in combination with one or more adjuvant compositions or treatments. In some cases, the one or more adjuvant treatments are selected from immunotherapy, virotherapy, targeted inhibition, radiotherapy, chemotherapy, or a combination thereof. In some cases, the subject is a mammal. In some cases, the subject is at risk of having a respiratory infection or a cancer. In some cases, the method is used to prevent the establishment of the disease in the subject. In some cases, the method is used to prevent progression of the disease in the subject. In some cases, the method is used to prevent the transmission of disease to a second subject.

### BRIEF DESCRIPTION OF THE DRAWINGS

A better understanding of the features and advantages of the present subject matter will be obtained by reference to the following detailed description that sets forth illustrative embodiments, in which the principles of the invention are utilized, and the accompanying drawings of which:

FIG. 1 provides a depiction of a composition comprising a liposome, a payload, and an antigen for treating or preventing a disease, in accordance with various embodiments of the present disclosure.

FIG. 2A illustrates a method of preventing a disease in a subject (e.g., an animal, such as a mammal) by administering one or more therapeutic compositions of the present disclosure to a subject before exposure to a pathogen. FIG. 2B illustrates a method of treating a disease in a subject by administering one or more therapeutic compositions of the present disclosure to a subject after exposure to a pathogen.

FIG. 3 shows a schematic of administration of compositions described herein comprising a liposome, a trimeric antigen, and a STING agonist for treating or preventing disease or administration of control compositions lacking the trimeric antigen and the STING agonist, in accordance with embodiments.

FIG. 4A shows a schematic of a trimeric S-protein incorporated into compositions for treating or preventing disease, in accordance with embodiments. FIG. 4B shows an image from a denaturing sodium dodecyl-sulfate polyacrylamide gel electrophoresis (SDS-PAGE) gel assay performed on a purified trimeric S-protein represented in FIG. 4A, in accordance with embodiments.

FIG. 5A shows a timeline of method steps for in vivo testing of animal subjects using compositions described herein for treating or preventing a disease in a subject, in accordance with embodiments.

FIG. 5B shows a timeline of body weight measurements for in vivo mouse subject testing with compositions described herein for treating or preventing a disease, in accordance with embodiments.

FIG. 5C shows a timeline of body weight measurements for in vivo mouse subject testing with compositions described herein comprising a liposome, a trimeric antigen, and a STING agonist versus control treatments using a composition without the trimeric antigen or STING agonist, in accordance with embodiments.

FIG. 5D and FIG. 5E show assessment of humoral immune responses using antigen-based IgG ELISA analysis of blood serum on day 7 (FIG. 5D) and day 15 (FIG. 5E) following administration of a composition described herein comprising a liposome, a trimeric antigen, and a STING agonist versus control treatments using a composition without the trimeric antigen or STING agonist, in accordance with embodiments.

FIG. 5F shows assessment of humoral immune responses using antigen-based IgG ELISA analysis of bronchoalveolar lavage fluid obtained from mice on day 15 following administration of a composition described herein comprising a liposome, a trimeric antigen, and a STING agonist versus control treatments using a composition without the trimeric antigen or STING agonist, in accordance with embodiments.

FIG. 5G shows time-dependent kinetics of humoral immune responses in treated mice using antibody titers of blood serum collected at day 7, day 15, day 21, and day 24 after administration of a composition described herein comprising a trimeric antigen and STING agonist, in accordance with embodiments. FIG. 5H shows longitudinal analysis of the data for each mouse subject presented in FIG. 5G.

FIG. 5-I shows a quantification of antibody secreting cells (ASC) secreting IgA, detected in samples collected from spleens following treatment with a composition described herein comprising a liposome, a trimeric antigen, and a STING agonist versus control treatments using a composition without the trimeric antigen or STING agonist, in accordance with embodiments. FIG. 5J shows a quantification of antibody secreting cells (ASC) secreting S-protein specific IgA, detected in samples collected from spleens following treatment with a composition described herein comprising a liposome, a trimeric antigen, and a STING agonist versus control treatments using a composition without the trimeric antigen or STING agonist, in accordance with embodiments.

FIG. 5K shows a quantification of S-protein specific IgA levels detected in animal subjects' serum at day 24 following treatment with a composition described herein comprising a liposome, a trimeric antigen, and a STING agonist versus control treatments using a composition without the trimeric antigen or STING agonist, in accordance with embodiments.

FIG. 5L shows a quantification of S-protein specific IgA levels detected in bronchoalveolar lavage fluid obtained from mice treated with a composition described herein comprising a liposome, a trimeric antigen, and a STING agonist versus control treatments using compositions without the trimeric antigen or STING agonist, in accordance with embodiments.

FIG. 5M shows ID50 levels for antibody responses determined from pseudovirus neutralization assay measurements of serum from mice treated with a composition described herein comprising a liposome, a trimeric antigen, and a STING agonist, in accordance with embodiments.

FIG. 5N shows ID50 levels for bronchoalveolar lavage fluid antibody responses determined from pseudovirus neutralization assay measurements of bronchoalveolar lavage fluid (BALF) from animal subjects treated with a composition described herein comprising a liposome, a trimeric antigen, and a STING agonist, in accordance with embodiments.

FIG. 5-O shows ELISpot assay data of interferon gamma (IFNγ) levels detected in samples obtained from spleens following treatment with a composition described herein comprising a liposome, a trimeric antigen, and a STING agonist, in accordance with embodiments. FIG. 5P shows protein-level mapping of conserved peptides (15mers) used for ELISpot interferon gamma quantification assays, in accordance with embodiments.

FIG. 6A shows an experimental method for performing single-cell RNA (scRNA) sequencing (scRNA-seq) on samples obtained from nasal-associated lymphoid tissue (NALT), in accordance with embodiments.

FIG. 6B, FIG. 6C, and FIG. 6D show flow cytometry gating strategies for isolation of live cells for use in single-cell RNA sequencing (scRNA-seq) assays, in accordance with embodiments. In some cases, cells are identified using a flow cytometry gate as shown in FIG. 6B, single cells are identified from the population of cells obtained with the gate shown in FIG. 6B by using the gate in FIG. 6C, and live single cells are obtained from the population of single cells obtained with the gate shown in FIG. 6C by using the gate in FIG. 6D.

FIG. 7A shows a uniform manifold approximation and projection (UMAP) analysis of immune cells in the nasal-associated lymphoid tissue (NALT) of animal subjects treated with compositions described herein comprising an antigen, a liposome, and a STING agonist, in accordance with embodiments. FIG. 7B, FIG. 7C, FIG. 7D show violin plots of relative expressions of B-cell markers CD19, Ms4a1, and CD79a, respectively, in immune cell populations obtained from animal subjects treated with compositions described herein comprising an antigen, a liposome, and a STING agonist, in accordance with embodiments. FIG. 7E, FIG. 7F, and FIG. 7G show violin plots of relative expressions of T-cell markers CD3d, CD3e, and CD3g, respectively, in immune cell populations obtained from animal subjects treated with compositions described herein comprising an antigen, a liposome, and a STING agonist, in accordance with embodiments. FIG. 7H, FIG. 7-I, and FIG. 7J show violin plots of relative expressions of NK-cell markers GzmB, Ncr1, and Ccl5, respectively, in immune cell populations obtained from animal subjects treated with compositions described herein comprising an antigen, a liposome, and a STING agonist, in accordance with embodiments. FIG. 7K, FIG. 7L, and FIG. 7M show violin plots of relative expressions of myeloid cell markers CD14, S100a9, and IL-1b, respectively, in immune cell populations obtained from animal subjects treated with compositions described herein comprising an antigen, a liposome, and a STING agonist, in accordance with embodiments.

FIG. 8A shows identification of B-cell subpopulations based on uniform manifold approximation and projection (UMAP) analysis of scRNA-seq assays performed on animal subjects treated with compositions described herein comprising an antigen, a liposome, and a STING agonist, in accordance with embodiments.

FIG. 8B shows a quantification of the relative frequencies of B-cell subpopulations identified using UMAP analysis of scRNA-seq assays performed on animal subjects treated with compositions described herein comprising an antigen, a liposome, and a STING agonist, in accordance with embodiments.

FIG. 8C, FIG. 8D, FIG. 8E, FIG. 8F, FIG. 8G, and FIG. 8H show violin plots of relative expressions of CD69, CD38, CD83, CXCR4, Zfp36, and Erg1, respectively, in B-cell subpopulations obtained from animal subjects treated with compositions described herein comprising an antigen, a liposome, and a STING agonist, in accordance with embodiments.

FIG. 9A, FIG. 9B, FIG. 9C, FIG. 9D, FIG. 9E, FIG. 9F, FIG. 9G, FIG. 9H, FIG. 9-I, FIG. 9J, FIG. 9K, FIG. 9L, and FIG. 9M show violin plots of relative expressions of CD19, Ms4a1, Ighm, Irf4, Pax5, Sec61b, Fosb, Jun, Tram1, Igha, EGR3, Casp3, and Fos, respectively, in B-cell subpopulations identified using UMAP analysis using cells obtained from animal subjects treated with compositions described herein comprising an antigen, a liposome, and a STING agonist, in accordance with embodiments.

FIG. 10A shows identification of T-cell subpopulations based on uniform manifold approximation and projection (UMAP) analysis of scRNA-seq assays performed on animal subjects treated with compositions described herein comprising an antigen, a liposome, and a STING agonist, in accordance with embodiments.

FIG. 10B shows a quantification of the relative frequencies of T-cell subpopulations identified using UMAP analysis of scRNA-seq assays performed on animal subjects treated with compositions described herein comprising an antigen, a liposome, and a STING agonist, in accordance with embodiments.

FIG. 10C, FIG. 10D, FIG. 10E, FIG. 10F, FIG. 10G, and FIG. 10H show violin plots of relative expressions of CD69, Il6ra, CD27, Nr4a1, Tcf7, and Lef1, respectively, in T-cell subpopulations obtained from animal subjects treated with compositions described herein comprising an antigen, a liposome, and a STING agonist, in accordance with embodiments.

FIG. 11A, FIG. 11B, FIG. 11C, FIG. 11D, FIG. 11E, FIG. 11F, and FIG. 11G show violin plots of relative expressions of CD4, CD8a, CD28, IL7R, Npm1, Fos, and Jun, respectively, in T-cell subpopulations identified using UMAP analysis using cells obtained from animal subjects treated with compositions described herein comprising an antigen, a liposome, and a STING agonist, in accordance with embodiments.

FIG. 12A shows a cell-cell interaction network produced using data obtained from scRNA-seq analysis of cells obtained from nasal-associated lymphoid tissues of animal subjects treated with compositions described herein comprising an antigen, a liposome, and a STING agonist, in accordance with embodiments. FIG. 12B shows predicted ligand-receptor interactions (from left to right: TNFRSF13C to TNFSF13B; ICOSLG to ICOS; BTLA to TNFSF14; CD40 to CD40LG; IL-21 receptor to IL-21; CXCL12 to CXCR4; CD28 to CD86; CD28 to CD80) between GC B-cell subpopulation cells and Tfh subpopulation cells within nasal-associated lymphoid tissues (NALT).

FIG. 13A shows a schematic of a monomeric S-protein incorporated into compositions described herein that can be useful in immunization (e.g., vaccination) of animal subjects, in accordance with embodiments. FIG. 13B shows an image of a denaturing SDS-PAGE gel used to characterize the monomeric S-protein depicted in FIG. 13A, in accordance with embodiments.

FIG. 13C shows IgG ELISA quantification of humoral immune responses in serum obtained from animal subjects on day 15 (“d15”) following treatment with compositions described herein comprising a liposome, a monomeric antigen, and a STING agonist versus control treatments using a composition without the monomeric antigen or STING agonist, in accordance with embodiments.

FIG. 13D shows ID50 levels for antibody responses determined from pseudovirus neutralization assay measurements of serum from animal subjects treated with a composition described herein comprising a liposome, a monomeric antigen, and a STING agonist, in accordance with embodiments (limit of detection (LoD) is shown with a dotted line; horizontal line represents the data point mean; p-values were computed using Mann-Whitney statistical analysis).

FIG. 13E shows ELISpot assay data of interferon gamma (IFNγ) levels detected in samples obtained from animal subjects' spleens or lungs following treatment with a composition described herein comprising a liposome, a monomeric antigen, and a STING agonist, in accordance with embodiments.

FIG. 13F shows that monomeric protein-specific IgA was detectable in nasal wash of two animal subjects treated with a composition described herein comprising a liposome, a monomeric antigen, and a STING agonist, in accordance with embodiments (limit of detection (LoD) is shown with a dotted line; horizontal line represents the data point mean).

FIG. 14 shows mean body weight of animal subjects treated with compositions described herein comprising a liposome, a monomeric antigen, and a STING antigen, in accordance with embodiments (error bars denote standard error).

FIG. 15 shows a quantification of antibody secreting cells (ASC) secreting S-protein specific IgA, detected in samples collected from animal subjects' spleens following treatment with a composition described herein comprising a liposome, a monomeric antigen, and a STING agonist versus control treatments using a composition without the monomeric antigen or STING agonist, in accordance with embodiments (horizontal lines indicate mean; error bars denote standard error).

FIG. 16 shows a schematic of method steps comprising administration of compositions described herein comprising a liposome, a nucleocapsid protein antigen, and a STING agonist to an animal subject for treating or preventing disease or administration of control compositions lacking the nucleocapsid protein antigen and the STING agonist, in accordance with embodiments

FIG. 17 shows induction of interferon response factor (IRF) responses in THP-1 cells isolated from animal subjects at 6 hours, 12 hours, or 24 hours following treatment with compositions described herein comprising liposomes, nucleocapsid protein, and cGAMP, in accordance with embodiments.

FIG. 18 shows a schematic of a SARS-Cov2 nucleocapsid protein useful in compositions for treating or preventing disease in an animal subject, in accordance with embodiments.

FIG. 19 shows an image from a denaturing sodium dodecyl-sulfate polyacrylamide gel electrophoresis (SDS-PAGE) gel assay performed on a purified SARS-Cov2 nucleocapsid protein (N-protein) represented in FIG. 18, in accordance with embodiments.

FIG. 20A shows fluorescence emission spectra of DNA-bound DiYO-1 in the presence of the nucleocapsid protein represented in FIG. 18 at nucleocapsid concentrations of 0 μM, 0.1 μM, 0.5 μM, in accordance with embodiments. FIG. 20B shows fluorescence emission spectra of DNA-bound DiYO-1 in the presence of a DNA binding positive control (branched-chain polyethyleneimine (PEI)) at various molar ratios (R) of PEI nitrogen to DNA phosphate), in accordance with embodiments.

FIG. 21A shows a timeline of steps for intranasal treatment or vaccination of a mouse subject and sample collection, in accordance with embodiments.

FIG. 21B shows IgG ELISA quantification of humoral immune responses in serum obtained from animal subjects on day 27 (“D27”) following treatment with compositions described herein comprising a liposome, 10 μg nucleocapsid protein (N-protein)) antigen, and a STING agonist (“NanoSTING-N10”) versus treatment with compositions described herein comprising a liposome, 20 μg nucleocapsid protein (N-protein)) antigen, and a STING agonist (“NanoSTING-N20”), and versus control treatments using a composition without the nucleocapsid protein antigen or STING agonist (“Control”), in accordance with embodiments. FIG. 21C shows IgG ELISA quantification of humoral immune responses in bronchoalveolar lavage fluid (BALF) obtained from animal subjects on day 27 (“D27”) following treatment with compositions described herein comprising a liposome, 10 μg nucleocapsid protein (N-protein)) antigen, and a STING agonist (“NanoSTING-N10”) versus treatment with compositions described herein comprising a liposome, 20 μg nucleocapsid protein (N-protein)) antigen, and a STING agonist (“NanoSTING-N20”), and versus control treatments using a composition without the nucleocapsid protein antigen or STING agonist (“Control”), in accordance with embodiments. FIG. 21D shows IgA ELISA quantification of humoral immune responses in serum obtained from animal subjects on day 27 (“D27”) following treatment with compositions described herein comprising a liposome, 10 μg nucleocapsid protein (N-protein)) antigen, and a STING agonist (“NanoSTING-N10”) versus treatment with compositions described herein comprising a liposome, 20 μg nucleocapsid protein (N-protein)) antigen, and a STING agonist (“NanoSTING-N20”), and versus control treatments using a composition without the nucleocapsid protein antigen or STING agonist, in accordance with embodiments. Bars and columns in FIGS. 21B, 21C, and 21D show median values, significance was asserted at *p<0.05; **p<0.01 using a Mann-Whitney test.

FIG. 22 shows a quantification of ELISpot analysis of interferon gamma (IFNγ) and interleukin-4 (IL4) for cells obtained from spleen and lung samples after treatment with compositions described herein comprising a liposome, 10 μg nucleocapsid protein (N-protein)) antigen, and a STING agonist (“NanoSTING-N10”) versus treatment with compositions described herein comprising a liposome, 20 μg nucleocapsid protein (N-protein)) antigen, and a STING agonist (“NanoSTING-N20”), and versus control treatments using a composition without the nucleocapsid protein antigen or STING agonist, in accordance with embodiments. Bars and columns show median values, significance was asserted at *p<0.05; **p<0.01 using a Mann-Whitney test.

FIG. 23 shows a flow cytometry gating strategy for quantification of additional marker expression in CD8-positive cells obtained from animal subjects following treatment with a composition described herein comprising a liposome, a nucleocapsid protein (N-protein) antigen, and a STING agonist, in accordance with embodiments.

FIG. 24A shows a flow cytometry quantification of CD137 expression in splenic cells that are also CD8-positive (CD8⁺) from animal subjects having been treated with compositions described herein comprising a liposome, 10 μg nucleocapsid protein (N-protein)) antigen, and a STING agonist (“NanoSTING-N10”) versus treatment with compositions described herein comprising a liposome, 20 μg nucleocapsid protein (N-protein)) antigen, and a STING agonist (“NanoSTING-N20”), and versus control treatments using a composition without the nucleocapsid protein antigen or STING agonist, in accordance with embodiments. Bars and columns show median values, significance was asserted at *p<0.05 a Mann-Whitney test. FIG. 24B shows flow cytometry data of CD137 expression in CD8-positive splenic cells obtained from animal subjects following treatment with control compositions described herein comprising a liposome lacking nucleocapsid protein antigen and STING agonist, in accordance with embodiments. FIG. 24C shows flow cytometry data of CD137 expression in CD8-positive splenic cells obtained from animal subjects following treatment with compositions described herein comprising a liposome, 10 μg nucleocapsid protein (N-protein)) antigen, and a STING agonist (“NanoSTING-N10”), in accordance with embodiments.

FIG. 24D shows flow cytometry data of CD137 expression in CD8-positive splenic cells obtained from animal subjects following treatment with compositions described herein comprising a liposome, 20 μg nucleocapsid protein (N-protein)) antigen, and a STING agonist (“NanoSTING-N20”), and versus control treatments using a composition without the nucleocapsid protein antigen or STING agonist, in accordance with embodiments.

FIG. 25A shows a flow cytometry quantification of granzyme B (GrB) expression in splenic cells that are also CD8-positive (CD8⁺) from animal subjects having been treated with compositions described herein comprising a liposome, 10 μg nucleocapsid protein (N-protein)) antigen, and a STING agonist (“NanoSTING-N10”) versus treatment with compositions described herein comprising a liposome, 20 μg nucleocapsid protein (N-protein)) antigen, and a STING agonist (“NanoSTING-N20”), and versus control treatments using a composition without the nucleocapsid protein antigen or STING agonist, in accordance with embodiments. Bars and columns show median values, significance was asserted at *p<0.05 a Mann-Whitney test. FIG. 25B shows flow cytometry data of granzyme B expression in CD8-positive splenic cells obtained from animal subjects following treatment with control compositions described herein comprising a liposome lacking nucleocapsid protein antigen and STING agonist, in accordance with embodiments. FIG. 25C shows flow cytometry data of granzyme B expression in CD8-positive splenic cells obtained from animal subjects following treatment with compositions described herein comprising a liposome, 10 μg nucleocapsid protein (N-protein)) antigen, and a STING agonist (“NanoSTING-N10”), in accordance with embodiments FIG. 25D shows flow cytometry data of granzyme B expression in CD8-positive splenic cells obtained from animal subjects following treatment with compositions described herein comprising a liposome, 20 μg nucleocapsid protein (N-protein)) antigen, and a STING agonist (“NanoSTING-N20”), and versus control treatments using a composition without the nucleocapsid protein antigen or STING agonist, in accordance with embodiments.

FIG. 26A shows a flow cytometry quantification of granzyme B (GrB) expression in lung T cells that are also CD8-positive (CD8⁺) from animal subjects having been treated with compositions described herein comprising a liposome, 10 μg nucleocapsid protein (N-protein)) antigen, and a STING agonist (“NanoSTING-N10”) versus treatment with compositions described herein comprising a liposome, 20 μg nucleocapsid protein (N-protein)) antigen, and a STING agonist (“NanoSTING-N20”), and versus control treatments using a composition without the nucleocapsid protein antigen or STING agonist, in accordance with embodiments. FIG. 26B shows a flow cytometry quantification of CD137 expression in lung T cells that are also CD8-positive (CD8⁺) from animal subjects having been treated with compositions described herein comprising a liposome, 10 μg nucleocapsid protein (N-protein)) antigen, and a STING agonist (“NanoSTING-N10”) versus treatment with compositions described herein comprising a liposome, 20 μg nucleocapsid protein (N-protein)) antigen, and a STING agonist (“NanoSTING-N20”), and versus control treatments using a composition without the nucleocapsid protein antigen or STING agonist, in accordance with embodiments. FIG. 26C shows a flow cytometry quantification of CD103 expression in lung T cells that are also CD8-positive (CD8⁺) from animal subjects having been treated with compositions described herein comprising a liposome, 10 μg nucleocapsid protein (N-protein)) antigen, and a STING agonist (“NanoSTING-N10”) versus treatment with compositions described herein comprising a liposome, 20 μg nucleocapsid protein (N-protein)) antigen, and a STING agonist (“NanoSTING-N20”), and versus control treatments using a composition without the nucleocapsid protein antigen or STING agonist, in accordance with embodiments. FIG. 26D shows a flow cytometry quantification of CD8-positive (CD8⁺) lung T cells that also express both CD69 and CD103 from animal subjects having been treated with compositions described herein comprising a liposome, 10 μg nucleocapsid protein (N-protein)) antigen, and a STING agonist (“NanoSTING-N10”) versus treatment with compositions described herein comprising a liposome, 20 μg nucleocapsid protein (N-protein)) antigen, and a STING agonist (“NanoSTING-N20”), and versus control treatments using a composition without the nucleocapsid protein antigen or STING agonist, in accordance with embodiments.

FIG. 27 shows a quantification of interferon response factor (IRF) responses in THP-1 dual cells in response to type I interferon inducer poly(dA:dT) complexed with cationic lipid-based transfection reagent LyoVec™ (InvivoGen) (“poly(dA:dT)/LV”) at 6 hours, 12 hours, and 24 hours after exposure to poly(dA:dT)/LV, in accordance with embodiments. Bars are shown in relative light units (RLU).

FIG. 28 shows measurements of animal subject body weight subjected to intranasally administered NanoSTING-N10 and NanoSTING-N20 compositions, in accordance with embodiments.

FIG. 29 shows nucleocapsid protein-reactive IgG ELISA quantifications of humoral immune responses in serum obtained from animal subjects at day 7 (“d7”), day 14 (“d14”), day 21 (“d21”), and day 27 (“d27”) following treatment with compositions described herein comprising a liposome, 10 μg nucleocapsid protein (N-protein)) antigen, and a STING agonist (“NanoSTING-N10”) and treatment with compositions described herein comprising a liposome, 20 μg nucleocapsid protein (N-protein)) antigen, and a STING agonist (“NanoSTING-N20”), in accordance with embodiments.

FIG. 30A shows ELISA quantification of IgG reactivity to alpha (“α”), beta (¢β∞), and gamma (“γ”) variants of a chimeric spike protein trimer, or to the receptor binding domain of the alpha or gamma variants, using serum samples obtained from animal subjects at day 28 following treatment with compositions described herein comprising a liposome, the chimeric spike protein trimer antigen, and a STING agonist (“NanoSTING-ChimS”) versus treatment with control compositions described herein comprising a liposome lacking the chimeric spike protein trimer antigen and the STING agonist, in accordance with embodiments. FIG. 30B shows ELISA quantification of IgA reactivity to the alpha variant of the chimeric spike protein trimer using serum samples obtained from animal subjects at day 28 following treatment with compositions described herein comprising a liposome, the chimeric spike protein trimer antigen, and a STING agonist (“NanoSTING-ChimS”) versus treatment with control compositions described herein comprising a liposome lacking the chimeric spike protein trimer antigen and the STING agonist, in accordance with embodiments.

FIG. 31A shows interferon gamma (IFNγ) responses, as quantified using ELISpot assay, in lung samples of animal subjects treated with NanoSTING-ChimS compositions, each comprising a different spike protein (S-protein) variant of the peptide pool and together representing all of the S-protein-derived peptide pool versus treatment with a control composition described herein comprising a liposome lacking a chimeric spike protein, in accordance with embodiments. FIG. 31B shows interferon gamma (IFNγ) responses, as quantified using ELISpot assay, in lung samples of animal subjects treated with NanoSTING-ChimS composition described herein comprising a liposome, a gamma variant chimeric spike protein trimer antigen, and a STING agonist versus treatment with a control composition described herein comprising a liposome lacking a chimeric spike protein, in accordance with embodiments. Bars and columns show median values, significance was asserted at *p<0.05; **p<0.01 using a Mann-Whitney test.

FIG. 32A and FIG. 32B show reactivity of IgG to B1 strain respiratory syncytial virus F protein (RSV-F) in ELISA assays performed using serum obtained from animal subjects at day 7 and day 14, respectively, following treatment with compositions described herein comprising a liposome, an RSV-F-derived antigen, and a STING agonist (“NanoSTING-RSV”) versus treatment with a control composition described herein comprising a liposome lacking a chimeric spike protein, in accordance with embodiments.

FIG. 33A, FIG. 33B, and FIG. 33C show distributions of liposomal particle sizes measured by dynamic light scattering (DLS), in accordance with embodiments.

FIG. 33D, FIG. 33E, and FIG. 33F show Zeta potential of liposomes measured by electrophoretic light scattering (ELS), in accordance with embodiments.

FIG. 34A and FIG. 34C show distributions of liposomal particle sizes measured by dynamic light scattering (DLS) after composition storage at 4° C., in accordance with embodiments.

FIG. 34B and FIG. 34D show Zeta potential of liposomes measured by electrophoretic light scattering (ELS) after composition storage at 4° C., in accordance with embodiments.

FIG. 35A and FIG. 35B show distributions of liposomal particle sizes measured by dynamic light scattering (DLS) where the antigen is stored in solution (FIG. 35A) or as a lyophilized solid (FIG. 35B), in accordance with embodiments.

FIG. 36A shows UV-visible absorption spectrum of cGAMP (STINGa) compositions, in accordance with embodiments.

FIG. 36B shows a standard curve used in calculating the concentration of free STINGa, in accordance with embodiments.

### DETAILED DESCRIPTION

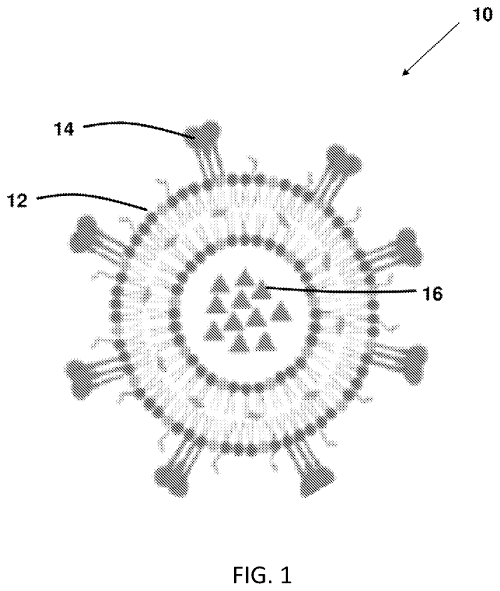

Disclosed herein are compositions and methods for the prevention and/or treatment of a disease or pathological condition of a subject (e.g., an animal, such as a mammal), such as a respiratory disease or a cancer. In some cases, preventing a disease or pathological condition comprises preventing the establishment of the disease or the pathological condition in a subject. In some cases, preventing a disease or pathological condition comprises preventing the establishment of the disease or the pathological condition in a subject (e.g., after exposure of the subject to a pathogen). In some cases, preventing a disease or pathological condition comprises preventing the development (e.g., progression) of the disease or the pathological condition (e.g., from a first state to a second, more severe or progressed state) in a subject (e.g., after establishment of a disease in the subject, for example, via infection from a pathogen). In some cases, preventing a disease or pathological condition comprises preventing the transmission of the disease or the pathological condition from a first subject to a second subject. A composition 10 useful for preventing or treating a disease or pathological condition of a subject can comprise a particle 12, a modulator 16, and/or an antigen 14 (e.g., as shown in FIG. 1). For example, compositions described herein can comprise a lipid-based liposome particle, modulator (such as a pattern recognition receptor agonist, e.g., a STING agonist), and an application-specific antigen (such as a coronavirus spike protein (“S-protein”), a nucleocapsid protein (“N-protein”), or a chimeric antigen protein) associated with the liposome particle. In some cases, association of an application-specific antigen with (e.g., an outer surface of) a liposome particle encapsulating a modulator payload can significantly increase the targeting and/or delivery of the modulator payload to a target tissue of interest (e.g., an intranasal compartment and/or a lung compartment of a subject). In some cases, association (e.g., direct association, such as through incorporation into the liposomal membrane or through coupling to the liposomal membrane surface) of an application-specific antigen with a liposome particle may increase the efficacy of the composition in preventing and/or treating a disease or pathological condition in a subject, for example, by spatially concentrating an antigen and a modulator of the composition (e.g., at a target tissue).

### Compositions

A composition (e.g., a therapeutic composition) for preventing or treating a disease or pathological condition in a subject can comprise a delivery vehicle (e.g., a particle). A delivery vehicle of a composition useful in preventing or treating a disease or pathological condition in a subject can be a particle, such as a lipid-based particle. For example, a composition useful in preventing or treating a disease or pathological condition in a subject can comprise a lipid-based particle, such as a liposome (e.g., having a membrane with an outer surface and an interior space). In some embodiments, the composition comprises one or more modulators (e.g., one or more types of modulators). In some cases, a modulator can comprise a small molecule, a protein or fragment thereof, and/or a polynucleotide or fragment thereof. In some cases, a modulator (e.g., a STING agonist) or combination of modulators can be selected for use in a composition described herein for its ability to induce activation or inhibition of a signaling pathway and/or a systemic response pathway (e.g., the stimulator of interferon genes (STING) pathway) in a subject. A composition useful in preventing or treating a disease or pathological condition in a subject can comprise one or more antigens. In some cases, an antigen of a composition described herein can elicit an immune response in a subject, which may be useful in preventing or abrogating a disease (e.g., a respiratory disease) or pathological condition (e.g., cancer) in the subject. In some embodiments, a composition for preventing or treating a disease or a pathological condition in a subject can comprise a modulator (e.g., a STING agonist, such as cGAMP) encapsulated within a particle (e.g., a liposome) wherein an antigen is associated with the liposome or a portion thereof (e.g., via incorporation into the liposomal membrane).

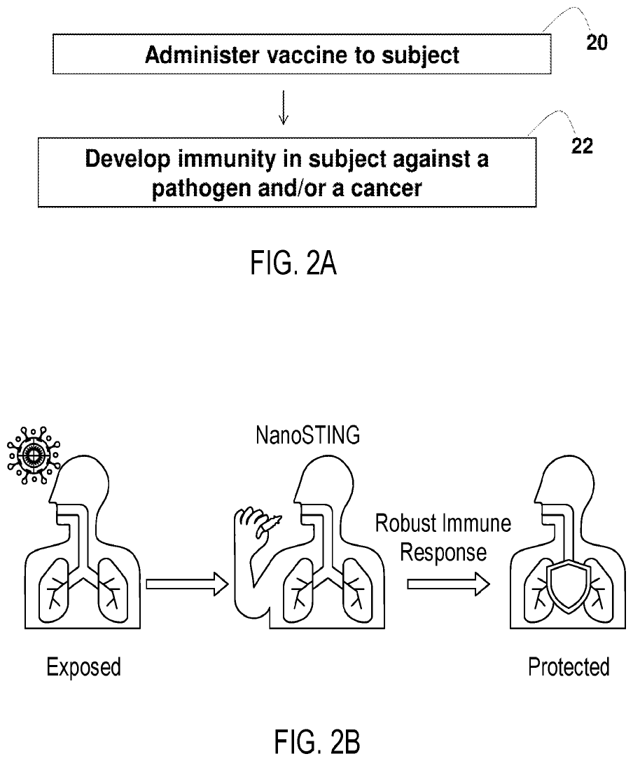

In some embodiments, a composition described herein can be used in a method for treating or preventing a disease or pathological condition in a subject (e.g., a mammal, such as a human). In some cases, a composition described herein can be administered to a subject before exposure to a pathogen (e.g., a pathogen causing a respiratory disease or cancer), for example, to prevent the subject from acquiring a disease or condition associated with exposure to (e.g., infection by) the pathogen (e.g., as illustrated in FIG. 2A). In some cases, a composition disclosed herein can be administered in the form of a vaccine. In some cases, a composition described herein can be administered to a subject after exposure to a pathogen (e.g., a pathogen causing a respiratory disease or cancer), for example, to treat (e.g., ameliorate or, in some cases, cure) a disease or condition associated with exposure to (e.g., infection by) the pathogen, for example, after the subject has acquired a disease or condition associated with the pathogen from exposure to the pathogen (e.g., as illustrated in FIG. 2B).

### Particles

A composition of the present disclosure can comprise a delivery vehicle, such as a particle. A particle can comprise a means of conveying a payload (e.g., a modulator payload) and/or one or more antigens (e.g., an antigen associated with a portion of the particle, such as a membrane or outer surface). In some cases, a particle can comprise a membrane or wall. In some cases, a membrane or wall of a particle can define an interior space. In some embodiments, an interior space of a particle can comprise one or more modulators. In some embodiments, one or more antigens can be associated with a membrane or wall of the particle (e.g., an outer surface of a membrane or wall of the particle). A particle can comprise a lipid-based particle, a carbon-based particle, a metal-based particle, or combinations thereof.

A particle of a composition described herein can be a lipid-based particle. In some embodiments, the lipid-based particle can be a liposome. A composition described herein can comprise a pulmonary surfactant-biomimetic particle. A particle of a composition disclosed herein (e.g., a lipid-based particle) can comprise a plurality of molecules, for example, assembled into a membrane. In some cases, a particle (e.g., a lipid-based particle) or a portion thereof can be anionic. In some cases, a particle (e.g., a lipid-based particle) or a portion thereof can be cationic. In some cases, a particle (e.g., a lipid-based particle can be zwitterionic. In some cases, a particle (e.g., a lipid-based particle) or a portion thereof can have a net zero charge. In some cases, a particle (e.g., a lipid-based particle) or a portion thereof can be uncharged. In some cases, a particle (e.g., liposome) of a composition described herein can comprise dipalmitoylphosphatidylcholine, dipalmitoylphosphatidylglycerol, 1,2-bis(diphenylphosphino)ethane (DPPE), cholesterol, or a combination thereof. In some embodiments, the a particle (e.g., liposome) of a composition described herein can comprise DPPC (1,2-dipalmitoyl-sn-glycero-3-phosphocholine), DPPG (1,2-dipalmitoyl-sn-glycero-3-phospho-(1′-rac-glycerol)), DPPE-PEG2000 (1,2-dipalmitoyl-sn-glycero-3-phosphoethanolamine-N-[methoxy(polyethylene glycol)-2000]), cholesterol, or a combination thereof. In some embodiments, a particle can comprise a combination of DPPC and DPPG, for example, at a molar ratio of about 10:1. In some cases, a particle can comprise a combination of DPPC and cholesterol, for example, at a molar ratio of about 10:1. In some cases, a particle can comprise a combination of DPPC and DPPE-2000, for example, at a molar ratio of about 10:1. In some cases, a particle can comprise a combination of DPPG and cholesterol, for example, at a molar ratio of about 1:1. In some cases, a particle can comprise a combination of cholesterol and DPPE-PEG2000, for example, at a molar ratio of about 1:1. In some cases, a particle can comprise a combination of DPPG and DPPE-PEG2000, for example, at a molar ratio of about 1:1. In some cases, a particle can comprise DPPC, DPPG, cholesterol, and DPPE-PEG2000. In some cases, a particle can be composed of a molar ratio of 10:1:1:1 of DPPC, DPPG, cholesterol, and DPPE-PEG2000, respectively. In some cases, a particle can be composed of a molar ratio of 20:1:1:1 of DPPC, DPPG, cholesterol, and DPPE-PEG2000, respectively. In some cases, a particle can be composed of a molar ratio of 5:1:1:1 of DPPC, DPPG, cholesterol, and DPPE-PEG2000, respectively. In some cases, a particle can be composed of a molar ratio of 10:2:1:1 of DPPC, DPPG, cholesterol, and DPPE-PEG2000, respectively. In some cases, a particle can be composed of a molar ratio of 10:1:2:1 of DPPC, DPPG, cholesterol, and DPPE-PEG2000, respectively. In some cases, a particle can be composed of a molar ratio of 10:1:1:2 of DPPC, DPPG, cholesterol, and DPPE-PEG2000, respectively. In some cases, a particle can be composed of a molar ratio of 10:2:2:1 of DPPC, DPPG, cholesterol, and DPPE-PEG2000, respectively. In some cases, a particle can be composed of a molar ratio of 10:1:2:2 of DPPC, DPPG, cholesterol, and DPPE-PEG2000, respectively. In some cases, a particle can be composed of a molar ratio of 10:2:1:2 of DPPC, DPPG, cholesterol, and DPPE-PEG2000, respectively. In some cases, a molar ratio of a first molecule comprising a particle (e.g., a lipid-based particle) to a second molecule comprising the particle can be from 1:100 to 1:1, from 1:50 to 1:2, from 1:25 to 1:3, from 1:10 to 1:5, from 1:50 to 1:1, from 1:25 to 1:1, from 1:10 to 1:1, from 1:5 to 1:1, from 1:3 to 1:1, from 1:25 to 1:10, from 1:50 to 1:25, from 1:100 to 1:50, or greater than 1:100. In some cases, a molar ratio of a first molecule comprising a particle (e.g., a lipid-based particle) of the present disclosure to a second molecule comprising the particle can be 1:1, 1:2, 1:3, 1:4, 1:5, 1:10, 1:15, 1:20, 1:25, 1:50, 1:100, or any range therebetween. In some cases, a molar ratio of a first molecule comprising a particle (e.g., a lipid-based particle) can be 1:5. In some cases, a molar ratio of a first molecule comprising a particle (e.g., a lipid-based particle) can be 1:10. In some cases, a molar ratio of a first molecule comprising a particle (e.g., a lipid-based particle) can be 1:20. In some cases, a molar ratio of a first molecule comprising a particle (e.g., a lipid-based particle) can be 1:50. In some cases, a first molecule comprising a particle (e.g., a lipid-based particle) can be 1:100. In some cases, a first molecule comprising a particle (e.g., a lipid-based particle) of a composition described herein can be dipalmitoylphosphatidylcholine, dipalmitoylphosphatidylglycerol, 1,2-bis(diphenylphosphino)ethane (DPPE), cholesterol, or a combination thereof. In some embodiments, a second molecule comprising a particle (e.g., lipid-based particle) of a composition described herein can be DPPC (1,2-dipalmitoyl-sn-glycero-3-phosphocholine), DPPG (1,2-dipalmitoyl-sn-glycero-3-phospho-(1′-rac-glycerol)), DPPE-PEG2000 (1,2-dipalmitoyl-sn-glycero-3-phosphoethanolamine-N-[methoxy(polyethylene glycol)-2000]), or cholesterol.

A particle of a composition described herein can be a nanoparticle. Maintaining a small particle diameter (e.g., mean hydrodynamic particle diameter less than 300 nanometers (nm), less than 200 nm, less than 150 nm, less than 120 nm, less than 115 nm, less than 111 nm, less than 110 nm, less than 105 nm, less than 100 nm, less than 95 nm, less than 90 nm, less than 85 nm, or less than 80 nm) and/or low polydispersity (e.g., polydispersity index less than 0.25, less than 0.24, less than 0.23, less than 0.22, less than 0.21, or less than 0.20) can improve particle stability and/or efficiency of delivery. In some cases, a particle can have an outer diameter of 1 nanometer to 500 nanometers, 1 nanometer to 750 nanometers, or 1 nanometer to 1,000 nanometers. In some cases, a particle can have an outer diameter of 1 nanometer to 10 nanometers, 1 nanometer to 15 nanometers, 1 nanometer to 20 nanometers, 1 nanometer to 30 nanometers, 1 nanometer to 50 nanometers, 1 nanometer to 75 nanometers, 1 nanometer to 100 nanometers, 1 nanometer to 150 nanometers, 1 nanometer to 200 nanometers, 1 nanometer to 250 nanometers, 1 nanometer to 300 nanometers, 1 nanometer to 400 nanometers, 1 nanometer to 500 nanometers, 10 nanometers to 15 nanometers, 10 nanometers to 20 nanometers, 10 nanometers to 30 nanometers, 10 nanometers to 50 nanometers, 10 nanometers to 75 nanometers, 10 nanometers to 100 nanometers, 10 nanometers to 150 nanometers, 10 nanometers to 200 nanometers, 10 nanometers to 250 nanometers, 10 nanometers to 300 nanometers, 15 nanometers to 20 nanometers, 15 nanometers to 30 nanometers, 15 nanometers to 50 nanometers, 15 nanometers to 75 nanometers, 15 nanometers to 100 nanometers, 15 nanometers to 150 nanometers, 15 nanometers to 200 nanometers, 15 nanometers to 250 nanometers, 15 nanometers to 300 nanometers, 20 nanometers to 30 nanometers, 20 nanometers to 50 nanometers, 20 nanometers to 75 nanometers, 20 nanometers to 100 nanometers, 20 nanometers to 150 nanometers, 20 nanometers to 200 nanometers, 20 nanometers to 250 nanometers, 20 nanometers to 300 nanometers, 30 nanometers to 50 nanometers, 30 nanometers to 75 nanometers, 30 nanometers to 100 nanometers, 30 nanometers to 150 nanometers, 30 nanometers to 200 nanometers, 30 nanometers to 250 nanometers, 30 nanometers to 300 nanometers, 50 nanometers to 75 nanometers, 50 nanometers to 100 nanometers, 50 nanometers to 150 nanometers, 50 nanometers to 200 nanometers, 50 nanometers to 250 nanometers, 50 nanometers to 300 nanometers, 75 nanometers to 100 nanometers, 75 nanometers to 150 nanometers, 75 nanometers to 200 nanometers, 75 nanometers to 250 nanometers, 75 nanometers to 300 nanometers, 100 nanometers to 150 nanometers, 100 nanometers to 200 nanometers, 100 nanometers to 250 nanometers, 100 nanometers to 300 nanometers, 150 nanometers to 200 nanometers, 150 nanometers to 250 nanometers, 150 nanometers to 300 nanometers, 200 nanometers to 250 nanometers, 200 nanometers to 300 nanometers, or 250 nanometers to 300 nanometers. In some cases, a particle can have an outer diameter of 1 nanometer, 10 nanometers, 15 nanometers, 20 nanometers, 30 nanometers, 50 nanometers, 75 nanometers, 100 nanometers, 150 nanometers, 200 nanometers, 250 nanometers, 300 nanometers, 400 nanometers, or 500 nanometers. In some cases, a particle can have an outer diameter of at least 1 nanometer, 10 nanometers, 15 nanometers, 20 nanometers, 30 nanometers, 50 nanometers, 75 nanometers, 100 nanometers, 150 nanometers, 200 nanometers, 250 nanometers, 300 nanometers, 400 nanometers, or 500 nanometers. In some cases, a particle can have an outer diameter of at most 1 nanometer, 10 nanometers, 15 nanometers, 20 nanometers, 30 nanometers, 50 nanometers, 75 nanometers, 100 nanometers, 150 nanometers, 200 nanometers, 250 nanometers, 300 nanometers, 400 nanometers, 500 nanometers, 750 nanometers, or 1,000 nanometers.

A modulator and/or an antigen of the present disclosure may be associated with the particles of the present disclosure. For instance, in some embodiments, the antigens and STING modulators of the present disclosure can be positioned on different regions of the particles of the present disclosure. In some embodiments, a modulator (e.g., STING modulator) of the present disclosure can be encapsulated in a particle (e.g., STING modulators 16 encapsulated in particle 12, as illustrated in FIG. 1). In some cases, all or a portion of an antigen of the present disclosure can be associated with (e.g., attached to, adhered to, adsorbed onto, covalently bound to, noncovalently bound to, integrated into) a surface of the particle (e.g., antigens 14 on surface of particle 12, as illustrated in FIG. 1). In some cases, all or a portion of an antigen is encapsulated within the lipid-based particle. In some embodiments, the association (e.g., adsorption) of an antigen with a surface of a particle can increase stability of the particle and/or increase delivery efficiency (e.g., to a target tissue) after administration.

In some cases, one or more antigens of the present disclosure can be encapsulated within a (e.g., lipid-based) particle of the present disclosure. In some cases, one or more modulators (e.g., including a STING modulator) of the present disclosure can be associated with an outer surface of the particle. In some embodiments, one or more antigens of the present disclosure can be encapsulated in a (e.g., lipid-based) particle while one or more modulator(s) (e.g., including a STING modulator) of the present disclosure are associated with an outer surface of the particle. In some cases, one or more antigens of the present disclosure can be associated with an outer surface of a (e.g., lipid-based) particle of this disclosure. In some cases, one or more modulators (e.g., including a STING modulator) of the present disclosure can be encapsulated within the particle. In some embodiments, one or more antigens of the present disclosure can be associated with an outer surface of a (e.g., lipid-based) particle of this disclosure while one or more modulators (e.g., including a STING modulator) of the present disclosure are encapsulated within the particle. In some embodiments, one or more antigens and one or more modulators (e.g., including a STING modulator) of the present disclosure can both be encapsulated within a (e.g., lipid-based) particle. In some embodiments, one or more antigens and one or more modulators (e.g., including a STING modulator) of the present disclosure may both be associated with a surface of a (e.g., lipid-based) particle of the present disclosure. In some cases, one or more antigens of the present disclosure can be integrated into a membrane of a (e.g., lipid-based) particle of the present disclosure. In some cases, one or more modulators (e.g., including a STING modulators) of the present disclosure can be integrated into a membrane of a (e.g., lipid-based) particle of the present disclosure. In some cases, one or more antigens of the present disclosure can be encapsulated within a (e.g., lipid-based) particle of the present disclosure while one or more modulators (e.g., including a STING modulator) are integrated into a membrane of a (e.g., lipid-based) particle of the present disclosure. In some cases, one or more antigens of the present disclosure can be associated with an outer surface of a (lipid-based) particle while one or more modulators (e.g., including a STING modulator) are integrated into a membrane of a (e.g., lipid-based) particle of the present disclosure. In some cases, one or more modulators (e.g., including a STING modulator) can be encapsulated within a (e.g., lipid-based) particle of the present disclosure while one or more antigens of the present disclosure are integrated into a membrane of the particle. In some cases, one or more modulators (e.g., including a STING modulator) can be associated with an outer surface of a (e.g., lipid-based) particle of the present disclosure while one or more antigens of the present disclosure are integrated into a membrane of the particle. In some embodiments, the incorporation of both an antigen and a modulator (e.g., a STING modulator) of the present disclosure within a (e.g., lipid-based) particle can facilitate coordinated cytosolic delivery.

### Antigens

A composition useful in preventing or treating a respiratory disease or pathological condition in a subject can comprise an antigen. An antigen (or a portion thereof) of the present disclosure can be associated with (e.g., attached to, adhered to, adsorbed onto, covalently bound to, noncovalently bound to, integrated into) a surface of the particle (e.g., a lipid-based particle, such as a liposome). In some cases, the antigen can be suitable for developing immunity in the subject against a disease. For instance, in some embodiments, one or more antigens in a composition of the present disclosure can be capable of eliciting an immune response in a subject against a disease.

An antigen of a composition described herein can include an attenuated or killed version of a pathogen, or portion thereof, that causes a disease (e.g., one or more of the pathogens described herein). In some embodiments, the antigen can comprise a peptide or a protein, or portion thereof, associated with the pathogen. In some embodiments, the antigen can comprise a surface protein (e.g., receptor protein), or portion thereof, of a pathogen. In some embodiments, the antigen may be in the form of a polynucleotide, for example, which may be used to express a second antigen (e.g., a plasmid DNA molecule expressing a second antigen).

An antigen of a composition described herein can comprise a spike protein or a portion thereof. In some cases, an antigen of a composition described herein can comprise at least one component of a coronavirus spike protein (S). In some cases, an antigen of a composition described herein can comprise a monomeric form of a coronavirus spike protein (S). In some cases, an antigen of a composition described herein can comprise a multimeric form of a coronavirus spike protein (S).

In some cases, an antigen of the present disclosure can comprise a monomeric form of the SARS-CoV2 spike protein (S), a monomeric form of the receptor binding domain (RBD) of the SARS-CoV2 spike protein (S), a multimeric form of the SARS-CoV2 spike protein (S), a multimeric form of the receptor binding domain (RBD) of the SARS-CoV2 spike protein (S), a dimeric form of the SARS-CoV2 spike protein (S), a dimeric form of the receptor binding domain (RBD) of the SARS-CoV2 spike protein (S), a trimeric form of the SARS-CoV2 spike protein (S), a trimeric form of the receptor binding domain (RBD) of the SARS-CoV2 spike protein (S), or combinations thereof. In some cases, an antigen of a composition can comprise a chimeric protein (e.g., a chimeric spike protein).

In some cases, an antigen can comprise a monomeric or multimeric form of the SARS-CoV2 spike protein (S) containing the D614G mutation, A222V mutation, S477N mutation, D80Y mutation, S98F mutation, or a combination thereof.

In some embodiments, the antigen can be a mixture of SARS-CoV2 spike proteins harboring different mutations. In some embodiments, the antigen can comprise the monomeric or multimeric form of a nucleocapsid protein. For example, an antigen of the present disclosure can comprise a monomeric or multimeric form of the SARS-CoV2 nucleocapsid (N) protein. In some embodiments, the antigen can be the monomeric or multimeric form of the SARS-CoV2 nucleocapsid (N) protein containing the A220V mutation.

In some embodiments, the antigen can be a mixture of SARS-CoV2 spike protein and nucleocapsid protein. In some embodiments, the antigen can be a mixture of SARS-CoV2 spike proteins harboring different mutations and a nucleocapsid protein.

In some cases, an antigen can be an influenza virus antigen or portion thereof, an influenza A virus antigen or portion thereof, an influenza B virus antigen or portion thereof, a parainfluenza virus antigen or portion thereof, an adenovirus antigen or portion thereof, an enterovirus antigen or portion thereof, a coronavirus antigen or portion thereof, a respiratory syncytial virus (RSV) antigen or portion thereof, a rhinovirus antigen or portion thereof, a DNA virus antigen or portion thereof, an RNA virus antigen or portion thereof, or a combination thereof.

In some embodiments, a composition of the present disclosure is suitable for developing immunity in the subject against a cancer. In some embodiments, an antigen of a composition of the present disclosure is suitable for developing immunity in the subject against a cancer. For instance, in some embodiments, the antigens in the therapeutic compositions of the present disclosure are capable of eliciting an immune response in a subject against a cancer (e.g., cancers described previously).

In some embodiments, an antigen of a composition of the present disclosure can be an attenuated or killed version of a tumor cell (or portion thereof) associated with a cancer. In some embodiments, the antigen can comprise a peptide or a protein associated with a cancer. In some embodiments, the antigen can comprise a surface protein (e.g., a receptor protein) of a cancer cell.

In some embodiments, the antigen can comprise a mutated protein of a cancer cell. In some embodiments, the antigen can be a synthetic long peptide targeting cancer mutations.

An antigen of a composition of the present disclosure can be in various forms. For instance, in some embodiments, the antigens can be recombinant peptides or proteins, or peptide epitopes recognized by T-cells. In some embodiments, antigens of a composition disclosed herein can comprise tandem minigenes. In some embodiments, the antigen can be in the form of a nucleotide expressing the antigen (e.g., a plasmid DNA molecule expressing the antigen).

### Modulators

A composition described herein for preventing or treating a disease in a subject (e.g., comprising a particle, such as a lipid-based particle, and, optionally, an antigen) can comprise a modulator. A modulator of a composition described herein can be a pattern recognition receptor modulator, such as a STING modulator (e.g., STING pathway modulator). In some cases, a modulator of a composition described herein can be a pattern recognition receptor agonist. In some cases, a modulator of a composition described herein can be a pattern recognition receptor antagonist. A composition of the present disclosure may include various types of STING modulators. For instance, in some embodiments, the STING modulator is an antagonist of the STING pathway. In some embodiments, the STING modulator is an agonist of the STING pathway. In some cases, a modulator of a composition described herein is capable of activating the STING pathway in a subject (e.g., to whom the composition is administered). In some cases, a modulator of a composition described herein is capable of inhibiting the STING pathway in a subject (e.g., to whom the composition is administered).

A STING modulator of a composition described herein can comprise bis-(3′,5′)-cyclic dimeric guanosine monophosphate (c-di-GMP), cyclic guanosine monophosphate-adenosine monophosphate (cGAMP), amidobenzimidazole, derivatives of amidobenzimidazole, nucleotide modulators, plasmid DNA modulators, nucleic acid modulators, or a combination thereof.

In some embodiments, the STING modulator can be an antagonist of the STING pathway. In some cases, a STING antagonist may be especially effective in treating and/or prevention of diseases, e.g., where a pathogen causing the disease (e.g., RNA viruses such as rhinoviruses) utilizes the STING pathway to promote viral replication. In some cases, a STING antagonist can be utilized to inhibit viral replication by reducing viral access to the STING pathway. In some embodiments, the STING antagonists include, without limitation, C-178, H-151, and combinations thereof.

In some cases, a modulator of a composition described herein can be disposed within an interior space of a particle of the composition. For instance, a modulator can be encapsulated within a particle (e.g., a lipid-based particle, such as a liposome) of a composition described herein. In some cases, a modulator of a composition described herein can be associated with a surface (e.g., an outer surface of a membrane) of a particle of the composition. In some cases, a modulator associated with a surface of a particle is associated by being covalently or non-covalently bound to the surface of the particle. In some cases, a modulator associated with the surface of the particle is associated by being integrated into the surface (e.g., membrane) of the particle.

In some cases, a nucleic acid sequence of an antigen of a composition described herein and a nucleic acid sequence of a modulator of the composition can be at least 85%, at least 90%, at least 95%, or 100% identical. In some cases, a portion of a nucleic acid sequence of an antigen of a composition described herein can be at least 85%, at least 90%, at least 95%, or 100% identical to a nucleic acid sequence of a modulator of the composition. In some cases, an antigen comprising a nucleic acid sequence that is at least 85%, at least 90%, at least 95%, or 100% identical to a nucleic acid sequence of a modulator described herein can be encapsulated within a particle (e.g., a lipid-based particle) of a composition described herein (e.g., wherein the modulator is also encapsulated within the lipid-based particle). In some cases, an antigen and a modulator described herein (e.g., wherein the antigen and the modulator comprise nucleic acid sequences that is at least 85%, at least 90%, at least 95%, or 100% identical) are both encapsulated within a lipid-based particle described herein. In some cases, a composition comprising an antigen and modulator comprising sequences that are at least 85%, at least 90%, at least 95%, or 100% identical can be delivered intranasally.

### Methods of Use

In additional embodiments, the present disclosure pertains to methods of treating or preventing a disease in a subject by administering the therapeutic compositions of the present disclosure to the subject. In more specific embodiments illustrated in FIG. 2A, the methods of the present disclosure include a step of administering a therapeutic composition to the subject (step 20) in order to treat or prevent a disease in the subject (step 22).

As set forth in more detail herein, the therapeutic compositions and methods of the present disclosure can have numerous embodiments. For instance, the therapeutic compositions of the present disclosure can include various types of STING modulators and antigens. Moreover, the methods of the present disclosure can be utilized to administer the therapeutic compositions of the present disclosure to numerous subjects in order to treat or prevent various diseases in various manners.

### Applications

In some cases, compositions and/or methods described herein may be useful in preventing or treating a respiratory disease or condition, such as a respiratory disease or condition related to influenza, coronavirus, respiratory syncytial viruses, rhinoviruses. In some cases, compositions and/or methods described herein can be useful in preventing or treating severe acute respiratory syndrome (SARS), acute respiratory distress syndrome (ARDS), and/or hypercytokinemia (e.g., cytokine storm). In some cases, compositions and/or methods described herein can be useful in preventing and/or treating a cancer, such as a lung cancer.

There is an urgent need for a safe and efficacious vaccine against respiratory diseases and cancer. In some cases, a composition described herein can comprise a vaccine or therapeutic treatment (e.g., delivered intranasally) containing a liposome containing agonists of the stimulator of interferon genes (STING) pathway to enable humoral immunity, T-cell immunity, systemic immunity and/or mucosal immunity.

Innate immunity is the first line of defense against invading pathogens. Innate immunity (unlike adaptive immunity, which can be customized for each pathogen) can be triggered by pattern recognition receptors (PRR) on the host cells that recognize conserved pathogen-associated molecular patterns (PAMPs)1. This mode of recognition can ensure that there is an immediate response that does not need customization.

In the context of RNA viruses, the recognition and activation of virus-specific RNA molecules can lead to the activation of interferon regulator factors (IRFs) and nuclear factor κB (NF-κB). Together, these transcriptional regulators launch broad antiviral programs, including the synthesis and secretion of type-I and type-III interferons and the subsequent upregulation of IFN-stimulated genes (ISGs).

This comprehensive antiviral program can apply a strong selection pressure on viral replication. Viruses have evolved elaborate countermeasures to interfere with interferon signaling. The interplay between the interferon mediated response and the viral countermeasures is heterogeneous and may be an explanation for the heterogeneity in morbidity and mortality seen in humans.

The type-1 interferon response can be suppressed in pathogen infections (e.g., coronavirus or other respiratory pathogens) and the balance between the ISGs and a pro-inflammatory response mediated by NF-κB can subsequently be dysregulated. Consequently, patients with advanced disease upon pathogen infection (e.g., respiratory pathogen infection) may have low interferon signaling but exaggerated tumor necrosis factor (TNF) and interleukin-6 (IL-6) secretion. Indeed, humans with autoantibodies against interferons that neutralize interferon function can be at high risk of developing advanced disease.

Similarly, humans with inborn errors of type I IFN immunity may develop life-threatening COVID-19 at a higher rate than those without such errors. In some cases, pretreatment of cell lines with type 1 interferon can inhibit viral replication in otherwise susceptible cells. Without being bound by theory, these considerations may help to explain that a lack of a robust type I IFN response can underlie advanced COVID-19.

The stimulator of interferon genes (STING) pathway is a PRR that senses cyclic DNA dinucleotides and activates IRF3 and NF-κB leading to the synthesis of ISGs. Although primarily thought to be important for sensing bacteria and DNA viruses, the role of STING in RNA virus-mediated type I IFN and cytokine production needs more detailed study. As shown herein, the role of STING in RNA virus-mediated type I IFN and cytokine production may be virus and cell type specific.

The sensing of double stranded RNA derived from viruses like the coronavirus within the cytoplasm of human cells can be accomplished by the RIG-I like receptors, including the retinoic acid inducible gene 1 (RIG-1) and melanoma differentiation gene 5 (MDA5). In some cases, the sensing pathways downstream of these receptors lead to the activation of the two IKK-related kinases, TANK-binding kinase 1 (TBK1) and inducible IκB kinase (IKKi). In some cases, these kinases are also engaged by the activated STING pathway, suggesting that while the PRRs have evolved to sense different molecules they may converge in their downstream responses.

Utilizing STING agonists as therapeutics can take advantage of this conserved downstream effector responses to promote a balanced activation of type I interferons, even in the context of SARS-CoV2 infection. Enabling the release of type I interferons with STING agonists independent of the RIG-1 like receptors increases the likelihood that this is not subjected to countermeasures evolved by the virus.

The release of interferons may enable a broad antiviral program limiting viral replication and thus mitigate disease severity. On the other hand, RNA viruses like rhinoviruses can hijack the STING pathway to promote viral replication, in some cases. In this context, it can be useful to inhibit viral replication by utilizing a STING antagonist to dampen inflammation.

Without being bound by theory, the therapeutic compositions of the present disclosure can be used to treat or prevent a disease through various mechanisms of action. For instance, in some embodiments, the therapeutic compositions of the present disclosure can be used to treat a disease in a subject. In some embodiments, the therapeutic compositions of the present disclosure can be used to prevent a disease in a subject. In some embodiments, the therapeutic compositions of the present disclosure can be used to treat and prevent a disease in a subject. In some embodiments, the therapeutic compositions of the present disclosure can be used to treat or prevent a disease in a subject by developing immunity in the subject against the disease.

For instance, in some embodiments, the therapeutic compositions of the present disclosure can elicit an immune response in the subject against the disease. In some embodiments, the therapeutic compositions of the present disclosure can elicit such immunity through at least one of innate immunity, mucosal immunity, systemic immunity, cellular immunity, humoral immunity, T-cell immunity, production of systemic neutralizing antibodies, induction of IgG responses, induction of IgA responses, induction of IgM responses, induction of T-cell responses, induction of mucosal IgA responses in lung and nasal compartments, induction of Th1 T-cell responses, induction of CD8+ T-cell responses, induction of CD4+ T cell responses, induction of NK cell responses, activation or inhibition of the stimulator of interferon genes (STING) pathway, or combinations thereof.

In some cases, a composition disclosed herein can elicit a mean dilution titer (e.g., of an IgG or an IgA specific to a pathogen antigen, or portion thereof) in the serum of a subject at least 1:25, greater than 1:25. In some cases, a composition disclosed herein can elicit a mean dilution titer (e.g., of an IgG or an IgA specific to a pathogen antigen, or portion thereof) in the bronchoalveolar lavage fluid (BALF) of a subject of from 1:25 to 1:50, from 1:30 to 1:50, from 1:40 to 1:50, from 1:50 to 1:60, or at least 1:25, greater than 1:25, or less than 1:60.

In some embodiments, compositions of the present disclosure can provoke or aid in the development of immunity against a disease in a subject, e.g., by eliciting an innate immune response in the subject against the disease. In some embodiments, the elicited innate immune response can lead to the activation of interferon regulator factors (IRFs), nuclear factor κB (NF-κB), or combinations thereof. In some embodiments, the elicited innate immune response can result in the synthesis and secretion of type-I and type-III interferons (IFNs) and the subsequent upregulation of IFN-stimulated genes (ISGs).

In some embodiments, the therapeutic compositions of the present disclosure can be used to develop immunity in a subject against a disease, e.g., through mucosal immunity and/or systemic immunity. In some embodiments, the therapeutic compositions of the present disclosure can aid in developing immunity in a subject through mucosal immunity, systemic immunity, and cellular immunity. In some embodiments, systemic immunity can be developed through the production of neutralizing antibodies against an antigen. In some embodiments, cellular immunity may be developed in spleen cells and/or lung cells. In some embodiments, mucosal immunity can be developed through production of IgA in the nasal compartment and lung, and IgA secreting cells in the spleen.

The methods and therapeutic compositions of the present disclosure can provide numerous advantages in various embodiments. For instance, in some embodiments, the therapeutic compositions of the present disclosure can be administered to large populations without the need for large clinical facilities.

Moreover, in some embodiments, the therapeutic compositions of the present disclosure can prevent establishment of an initial viral reservoir (e.g., in a nasal compartment) and may help to control viral dissemination between individuals and/or dissemination within an individual.

In some embodiments, the therapeutic compositions of the present disclosure can correct a deficiency in interferon activation, which may be one of the primary mechanisms of escape mediated by respiratory viruses. Additionally, the therapeutic compositions of the present disclosure can provide universal mucosal adjuvants for developing broad-spectrum therapeutic compositions against numerous pathogens, such as coronaviruses or other respiratory pathogens, e.g., because the therapeutic compositions of the present disclosure can be utilized in some embodiments to elicit one or more of IgM, IgG, IgA and T-cell responses in subjects.

In some embodiments, the methods and therapeutic compositions of the present disclosure can have veterinary applications. In some embodiments, the veterinary applications include the treatment or prevention of diseases in different types of animals and other similar applications.

### Formulation and Administration

Compositions of the present disclosure can be in various forms. In some cases, a composition described herein can be delivered as a solubilized liquid. In some embodiments, the therapeutic compositions of the present disclosure are suitable for intranasal and/or inhalational administration to a subject. In some cases, intranasal delivery of compositions disclosed herein can be used to target intranasal compartment tissues. In some cases, inhalational administration of compositions disclosed herein can be used to target lung compartment tissues.

In some cases, a syringe or liquid dropper can be used to administer a composition described herein intranasally to a subject. In some cases, administering a composition described herein to a subject can comprise the use of a spray nozzle, a nebulizer, and/or an atomizer. in some embodiments, the therapeutic compositions of the present disclosure can be self-administered.

In some embodiments, the therapeutic compositions of the present disclosure also include one or stabilizers. In some embodiments, the stabilizers include, without limitation, anti-oxidants, sequestrants, ultraviolet stabilizers, or combinations thereof.

In some embodiments, the therapeutic compositions of the present disclosure also include one or more surfactants. In some embodiments, the surfactants include, without limitation, anionic surfactants, sugars, cationic surfactants, zwitterionic surfactants, non-ionic surfactants, or combinations thereof.

In some embodiments, the therapeutic composition of the present disclosure also includes one or more excipients. In some embodiments, the excipients include, without limitation, lactose, sucrose, starch powder, cellulose esters of alkanoic acids, trehalose, cellulose alkyl esters, talc, stearic acid, magnesium stearate, magnesium oxide, sodium and calcium salts of phosphoric and sulfuric acids, gelatin, acacia gum, trehalose, sodium alginate, polyvinylpyrrolidone, polyvinyl alcohol, or combinations thereof.

The therapeutic compositions of the present disclosure can be administered by various methods. For instance, in some embodiments, the administration occurs by methods that include, without limitation, intravenous administration, intramuscular administration, intradermal administration, intraperitoneal administration, subcutaneous administration, spray-based administration, aerosol-based administration, in ovo administration, oral administration, intraocular administration, intratracheal administration, intranasal administration, inhalational administration, or combinations thereof.

In some embodiments, the therapeutic compositions of the present disclosure are administered in a single dose. In some embodiments, the therapeutic compositions of the present disclosure are administered in multiple doses. In some cases, a method of treating or preventing a disease in a subject (e.g., an animal subject) can comprise administering 1, 2, 3, 4, 5, 6, 7, 8, 9, 10, 11, 12, 13, 14, 15, 15 to 20, 20 to 25, 25 to 30, 30 to 40, 40 to 50, or more than 50 doses. In some cases, a composition of the present disclosure may be administered to a subject (e.g., an animal subject) at a In some cases, a method of the present disclosure can comprise administering to a subject a first dose comprising a composition of the present disclosure and a second dose comprising a composition of the present disclosure. In some cases, the composition of the first dose is the same as the composition of the second dose. In some cases, the composition of the first dose is different than the composition of the second dose. In some cases, a method of the present disclosure can comprise administering the first dose and the second dose to a subject (e.g., an animal subject) at an interval of at least 6 hours, at least 12 hours, at least 24 hours, at least 36 hours, at least 2 days, at least 3 days, at least 4 days, at least 5 days, at least 6 days, at least 7 days, at least 14 days, or at least 28 days. In some cases, a method of the present disclosure can comprise administering the first dose and the second dose to a subject (e.g., an animal subject) at an interval of at most 6 hours, at most 12 hours, at most 24 hours, at most 36 hours, at most 2 days, at most 3 days, at most 4 days, at most 5 days, at most 6 days, at most 7 days, at most 14 days, or at most 28 days. In some cases, a method of the present disclosure can comprise administering the first dose and the second dose to a subject (e.g., an animal subject) at an interval of 6 hours, 12 hours, 24 hours, 36 hours, 2 days, 3 days, 4 days, 5 days, 6 days, 7 days, 14 days, or 28 days. In some cases, a method of the present disclosure can comprise administering each dose subsequent to a first dose of a plurality of doses (e.g., wherein each dose of the plurality of doses comprises one or more compositions of the present disclosure) to a subject (e.g., an animal subject) at least 6 hours, at least 12 hours, at least 24 hours, at least 36 hours, at least 2 days, at least 3 days, at least 4 days, at least 5 days, at least 6 days, at least 7 days, at least 14 days, or at least 28 days after administration of the previous dose. In some cases, a method of the present disclosure can comprise administering each dose subsequent to a first dose of a plurality of doses (e.g., wherein each dose of the plurality of doses comprises one or more compositions of the present disclosure) to a subject (e.g., an animal subject) at most 6 hours, at most 12 hours, at most 24 hours, at most 36 hours, at most 2 days, at most 3 days, at most 4 days, at most 5 days, at most 6 days, at most 7 days, at most 14 days, or at most 28 days. In some cases, a method of the present disclosure can comprise administering each dose subsequent to a first dose of a plurality of doses (e.g., wherein each dose of the plurality of doses comprises one or more compositions of the present disclosure) to a subject (e.g., an animal subject) 6 hours, 12 hours, 24 hours, 36 hours, 2 days, 3 days, 4 days, 5 days, 6 days, 7 days, 14 days, or 28 days.

In some cases, a compositions described herein (e.g., “NanoSTING” compositions comprising a lipid-based particle, a modulator, and an antigen) can be formulated and/or administered as a monotherapy (e.g., for treating a disease or condition in a subject, for example, which has resulted from exposure to a pathogen).

In some embodiments, the therapeutic compositions of the present disclosure are administered in combination with other therapeutic treatments. In some embodiments, the other therapeutic treatments include, without limitation, immunotherapy, virotherapy, targeted inhibition, radiotherapy, chemotherapy, or combinations thereof. In some cases, a composition described herein can comprise and/or can be administered in a treatment regimen (e.g., administered concurrently or non-concurrently) with an adjuvant (e.g., one or more antibodies, one or more antiviral treatments, one or more vaccines, one or more small molecules, one or more nucleic acids, and/or one or more peptides or proteins, such as an interleukin (e.g., IL-21)).

### Subjects

The therapeutic compositions of the present disclosure may be administered to subjects in need thereof. For instance, in some embodiments, the subject is a mammal (e.g., a human). In some embodiments, the subject is a domesticated animal. For example, the subject can be a dog or a cat. In some cases, a subject can be a cow, a horse, a non-human primate, a mouse, a rat, a rabbit, a guinea pig, a goat, a sheep, a giraffe, a zebra, a lion, a tiger, or a bear. In some embodiments, the subject is a human being. In some embodiments, the subject is vulnerable to or suffering from a respiratory infection. In some embodiments, the subject is vulnerable to or suffering from a cancer. In some cases, a subject can be selected for treatment as a result of exhibiting one or more symptoms of infection from a pathogen or contraction of a disease (e.g., infection by a respiratory pathogen or contraction of a respiratory disease). For example, a subject may be selected by for treatment based on having one or more symptoms, including persistent coughing, elevated body temperature (e.g., greater than 100.4° C. by forehead skin measurement), body chills, achy joints, difficulty breathing or catching one's breath, fluid in the lungs, fatigue, headache, loss of taste or smell, or a positive diagnostic test, such as a PCR test. In some cases, a subject may be selected for treatment with a composition disclosed herein based on a demographic risk factor, such as obesity, advanced age (e.g., 65 years old or older), immune impairment, or pregnancy. In some cases, a subject may be selected for treatment based on a risk of infection by a pathogen or contraction of a disease (e.g., infection by a respiratory pathogen or contraction of a respiratory disease), for instance, if the subject has an occupation involving close interaction with customers, frequent interaction with at-risk populations, handling of biological samples, or close contact with potentially infected individuals.

### Treatment or Prevention of Diseases

Pathogens and cancers have caused significant health and economic concerns. For instance, viral infections caused by severe acute respiratory syndrome-related coronavirus 2 (SARS-CoV-2) have resulted in the COVID-19 pandemic, which is currently the most urgent health and economic crisis in the world. Moreover, cancers such as lung cancer have high fatality rates.

Additionally, very limited therapeutic options exist for effectively treating and preventing cancers, and infections caused by pathogens. For instance, no medicine is currently available for treating or preventing viral infections caused by SARS-CoV-2.

Additionally, many vaccines that aim to prevent infections caused by pathogens are intramuscular vaccines that are designed to elicit systemic immunity without conferring innate immunity, such as mucosal immunity or broad T-cell immunity. Such intramuscular vaccines present limitations because the nasal compartment is the first barrier that needs to be breached by pathogens before dissemination to the lung.

Furthermore, a high level of unpredictability exists in the effectiveness of vaccine formulations. For vaccines that make it to Phase I trials, only ˜⅓ are able to obtain eventual FDA approval. This rate of success implies that, even with existing technologies to prime the immune system, the development of effective immunity to every pathogen requires design and optimization of the correct antigen and the adjuvant.

Even achieving FDA approval does not imply that a vaccine is efficacious. Developing vaccines is a lengthy process and, even in the context of the COVID19 pandemic, there are a number of challenges.

Even though the majority of platforms target the spike protein as the antigen, optimizing the antigen design is desirable to ensure the efficacy of the vaccine. The difficulty in picking the appropriate vaccine design has been highlighted with HIV-1, Hepatitis C and malaria, wherein, despite decades of efforts and multiple candidates, vaccines remains elusive.

Moreover, with respect to respiratory pathogens, the nasal compartment is the first barrier that needs to be breached by the pathogens before dissemination to the lung. However, current intramuscular vaccines are designed to elicit systemic immunity without conferring innate immunity, such as mucosal immunity.

Accordingly, safe and durable therapeutic compositions are urgently needed to treat and prevent various diseases, such as infections caused by pathogens and various types of cancer. For instance, safe and durable therapeutic compositions are urgently needed to treat or prevent pandemics caused by respiratory pathogens (e.g., viral infections caused by coronaviruses, such as SARS-CoV-2). Numerous embodiments of the present disclosure address the aforementioned needs.

The therapeutic compositions and methods of the present disclosure can be utilized to treat or prevent a disease or condition (e.g., a disease or condition associated with a pathogen). For instance, in some embodiments, the disease can be an infection caused by a pathogen. In some cases, the disease can be a respiratory disease. In some embodiments, the pathogen can be a respiratory pathogen. In some embodiments, the respiratory pathogen can be a virus. In some embodiments, the virus can be an influenza virus, an influenza A virus, an influenza B virus, a parainfluenza virus, an adenovirus, an enterovirus, a coronavirus, a respiratory syncytial virus, a rhinovirus, a DNA virus, an RNA virus, or a combination thereof.

In some embodiments, the pathogen is a coronavirus. In some embodiments, the coronavirus includes, without limitation, severe acute respiratory syndrome coronavirus (SARS-CoV), severe acute respiratory syndrome-related coronavirus (SARSr-CoV), human coronavirus 229E (HCoV-229E), human coronavirus NL63 (HCoV-NL63), human coronavirus OC43 (HCoV-OC43), human coronavirus HKU1 (HCoV-HKU1), Middle East respiratory syndrome-related coronavirus (MERS-CoV), severe acute respiratory syndrome-related coronavirus 2 (SARS-CoV-2), variants of SARS-CoV-2 (e.g., 20A.EU1, or spike variant D614G), or combinations thereof.

In some embodiments, the disease to be treated or prevented in a subject is a cancer. In some embodiments, the cancer can be tracheal cancer, lung cancer, bronchial cancer, epithelial cancer, blood cancer, breast cancer, melanoma, ovarian cancer, gynecological cancer, a leukemia, a lymphoma, prostate cancer, bladder cancer, colon cancer, a glioma, a sarcoma, glioblastoma, or a combination thereof. In some cases, the cancer can be lung cancer.

### EXAMPLES

### Example 1. Preparation and Characterization of Lipid-Based Particles Comprising

This example describes the preparation and characterization of lipid-based particles, comprising a STING agonist modulator and a spike protein antigen, useful in compositions for treating or preventing a disease (e.g., a respiratory disease) in a subject. To facilitate efficient priming of the immune system within the respiratory compartment, the STING agonist was encapsulated within negatively charged liposomes to yield compositions comprising a liposome, a STING agonist modulator, and an antigen associated with the surface of the liposome (“NanoSTING”) (see, FIG. 1).

The liposomes were composed of a molar ratio of 10:1:1:1 of DPPC, DPPG, cholesterol, and DPPE-PEG2000. 1,2-dipalmitoyl-sn-glycero-3-phosphocholine (DPPC), 1,2-dipalmitoyl-sn-glycero-3-phospho-(1′-rac-glycerol) (DPPG), and 1,2-dipalmitoyl-sn-glycero-3-phosphoethanolamine-N-[methoxy(polyethylene glycol)-2000] (DPPE-PEG2000) were obtained from Avanti Polar Lipids (Alabaster, Ala.). Cholesterol was obtained from Sigma Aldrich (St. Louis, Mo.). To prepare the liposomes, the lipids were mixed in chloroform (CHCl₃) and methanol (CH₃OH), and the solution was evaporated by a vacuum rotary evaporator for approximately 80 minutes (min) at 45° C. The resulting lipid thin films were dried until all organic solvent was evaporated. The lipid film was hydrated by adding a pre-warmed cGAMP solution (0.3 mg/ml cGAMP in PBS buffer at pH 7.4). The STING agonist, 2′-3″cyclic guanosine monophosphate adenosine monophosphate (cGAMP) was obtained from Chemietek (Indianapolis, Ind.).

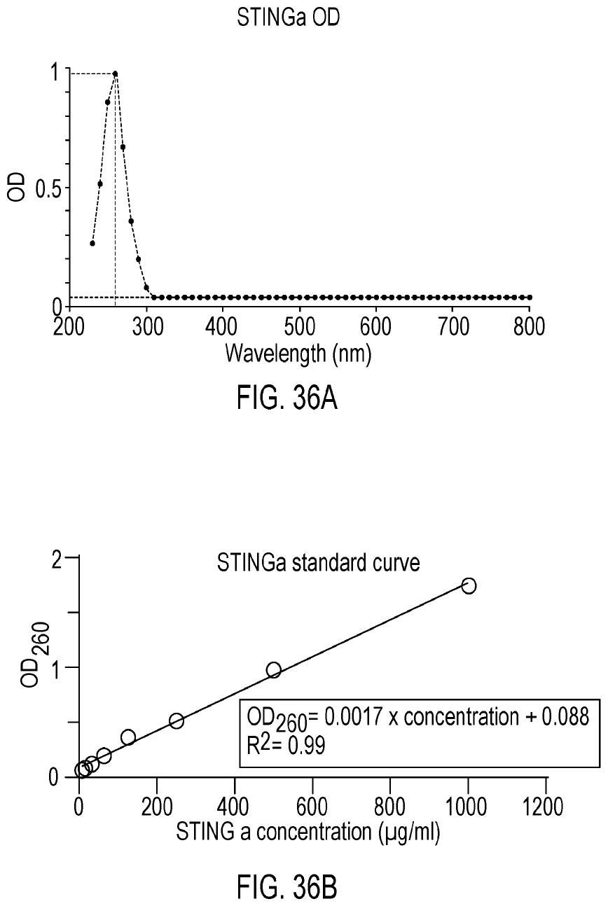

The hydrated lipids were mixed at elevated temperature 65° C. for an additional 30 min, then subjected to freeze-thaw cycles. The mixture was sonicated at 40 kHz for 60 min using a Branson Sonicator. The free untrapped cGAMP was removed by Amicon Ultrafiltration units using a molecular weight cut off of 10 kDa. The cGAMP-liposomes were washed three times using PBS buffer. The cGAMP concentration in the filtrates was measured by Take3 Micro-Volume absorbance analyzer of Cytation 5 (BioTek) against a calibration curve of cGAMP at 260 nm (FIG. 36A). The final concentration of liposomal encapsulated cGAMP and encapsulation efficiency were calculated by subtracting the concentration of free drug in the filtrate (standard curve shown in FIG. 36B).

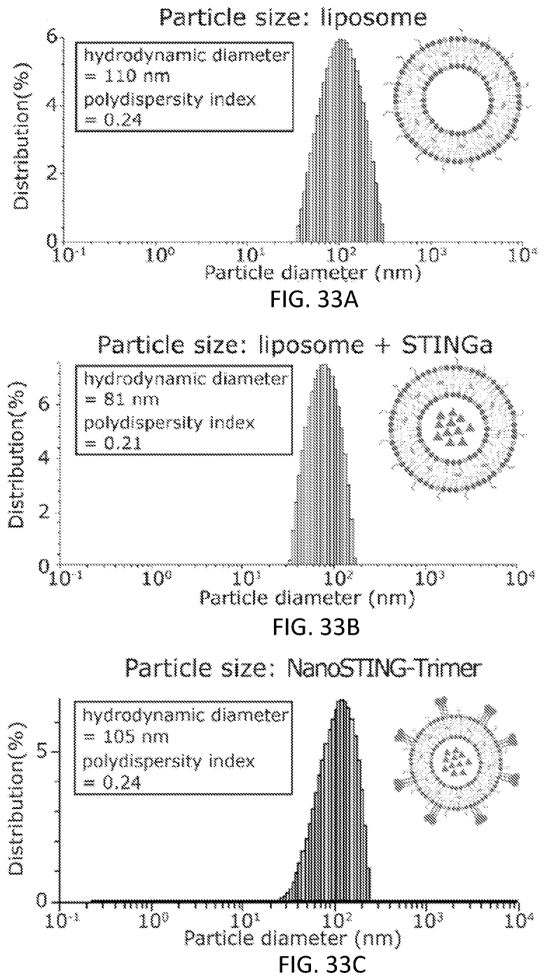

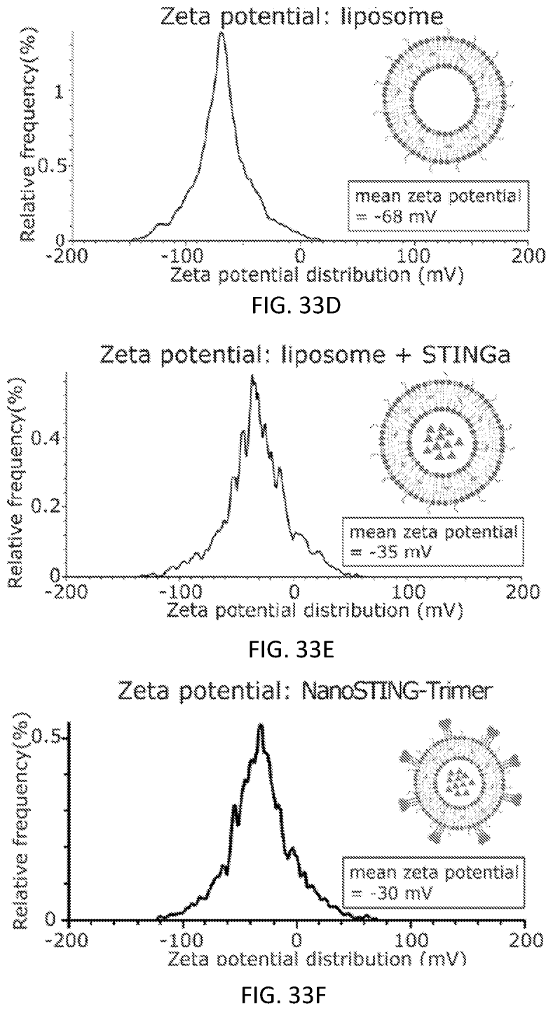

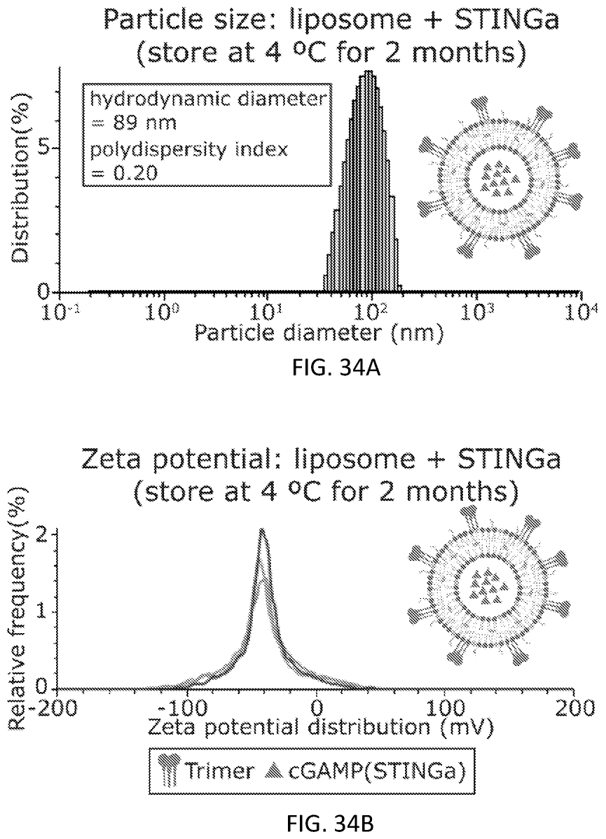

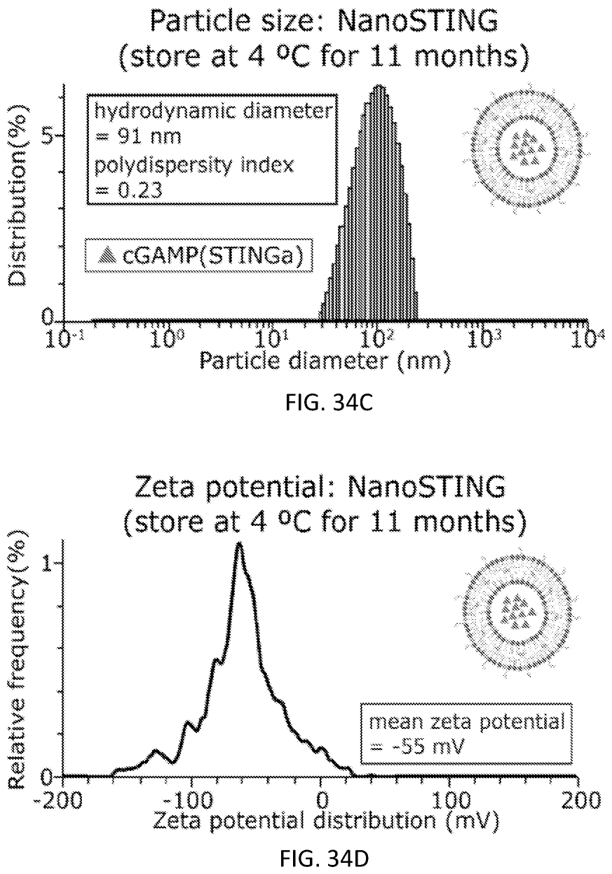

Freshly hydrated lyophilized form of SARS-CoV-2 spike protein (“S-protein”) monomer or, alternatively, trimer was mixed with the STINGa-liposomal suspensions at room temperature for 10 min to allow the adsorption of the protein onto the liposomes. The formulated composition was stored at 4° C. and used for up to 2 months. For stability determination, the STING agonist-liposomal suspensions were stored at 4° C. for 11 months. The average particle diameter, polydispersity index, and zeta potential were characterized by Litesizer 500 at room temperature. Dynamic light scattering (DLS) analysis showed that the mean particle diameter of NanoSTING compositions comprising a liposome and a STING agonist (“STINGa”) modulator, cGAMP, was 81 nm, with a polydispersity index of 0.21 (FIG. 33B), while the size of blank liposomes lacking STING agonist modulator or spike protein antigen was 110 nm (FIG. 33A). The mean particle diameter of NanoSTING compositions comprising a liposome encapsulating the STING agonist modulator and comprising S-protein trimer associated with the liposome's outer surface was 105 nm, with a polydispersity index of 0.24 (FIG. 33C). The mean zeta potential of liposomes was negative both with (−35 mV) (FIG. 33E) and without (−68 mV) (FIG. 33D) encapsulated STING agonist. For NanoSTING compositions comprising a liposome encapsulating the STING agonist modulator and comprising S-protein trimer associated with the liposome's outer surface. The mean zeta potential of the liposomes was −30 mV (FIG. 33F). The stability of the NanoSTING was tested, and data showed that they were stable after storage for 2 months at 4° C. (FIG. 34A) and for more than 11 months at 4° C., as evidenced by the conservation of particle sizes and the absence of aggregates (FIG. 34C). The surface charge of liposomes was also unaltered (−55 mV) after 2 months at 4° C. (FIG. 34B) and after 11 months at 4° C. (FIG. 34D). Collectively, these results established that the negatively charged liposomes had efficiently encapsulated the STINGa and had excellent stability.

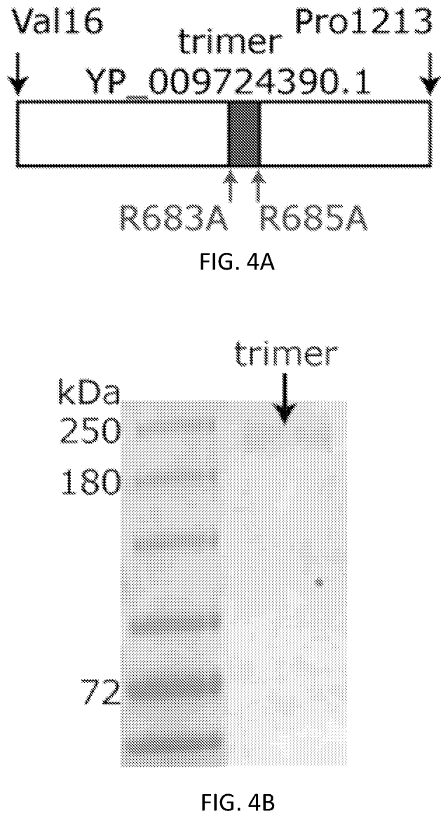

We used the lyophilized recombinant trimeric extracellular domain of the S-protein containing mutations to the Furin cleavage site as the immunogen (FIG. 4A). As expected by extensive glycosylation of the S-protein, SDS-PAGE under reducing conditions confirmed that the protein migrated between 180-250 kDa (FIG. 4B).

A single-step mix and immunize approach was used to prepare a NanoSTING trimeric S-protein composition (NanoSTING-Trimer) formulation comprising the trimeric S-protein and the liposome encapsulating the STING modulator. The NanoSTING suspension was gently mixed with the freshly rehydrated Trimeric S-protein with gentle agitation at room temperature to allow the adsorption of the protein on the liposomes. The adsorbed trimeric S protein (NanoSTING-Trimer) displayed a mean particle diameter of 105 nm (FIG. 33C) and a mean zeta potential of −30 mV (FIG. 33F), with a polydispersity index of 0.24, slightly bigger and less negative than the NanoSTING (81 nm, −35 mV). Collectively, these results indicated that the components of the composition are stable and are easily formulated in a stable nanoparticulate colloidal form.

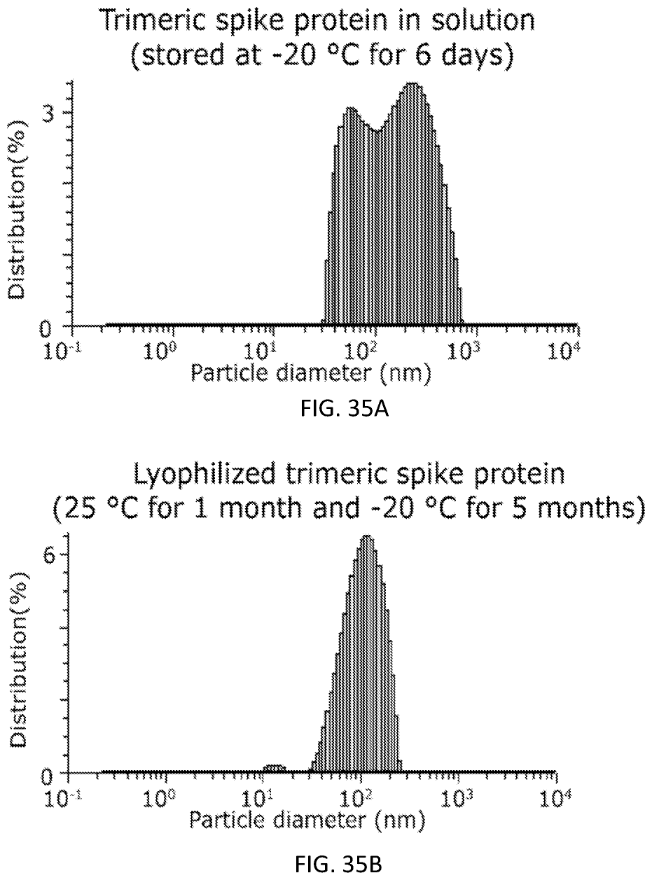

Because the trimeric form of the S-protein can aggregate in solution in some cases, two different formulations of the trimeric S-protein were tested: the lyophilized form and the solution form stored at −20° C. The lyophilized form even when stored for six months (25° C. for 1 month and −20° C. for 5 months) showed no evidence of aggregation once hydrated and measured immediately by DLS (FIG. 35B) whereas the solution form of the trimeric S-protein even after a week at −20° C. showed the presence of aggregated protein (presence of second particle peak >100 nm, polydispersity index=0.28) (FIG. 35A). These results illustrate that the lyophilized protein does not aggregate and can form the basis of a stable formulation.

### Example 2. Quantitative Enzyme-Linked Immunosorbent Assay (ELISA) Analysis of NanoSTING-S Compositions

This example shows the use of S-protein quantitative ELISA to measure S-protein adsorption on liposome particles of NanoSTING compositions.

Anti-S-protein antibody titers in serum or other biological fluids were determined using ELISA. Briefly, 1 μg/ml spike protein (Sino Biological, PA, USA) was coated onto ELISA plates (Corning, N.Y., USA) in PBS overnight at 4° C. or 2 hours at 37° C. The plate was blocked with PBS+1% BSA (Fisher Scientific, PA, USA)+0.1% Polysorbate 20 (Tween20™ from Sigma-Aldrich, MD, USA) for 2 hours at room temperature. After washing, samples prepared as in Example 1 were added at different dilutions. Captured antibodies were detected by horseradish peroxidase (HRP)-conjugated anti-mouse IgG (Jackson ImmunoResearch Laboratories, 1 in 5,000; PA, USA), anti-mouse IgA (Bethyl Laboratories, 1 in 10,000; TX, USA), and detection antibody against mouse IgA (1 in 250) from the mouse total IgA ELISA kit from Invitrogen (CA, USA). The positive control (anti-S IgG) was obtained from Abeomics (CA, USA).

To estimate the fraction of the protein adsorbed onto the liposomes, trimeric S-protein (10 μl, 1 μg/μl) was gently mixed with NanoSTING (20 μg), and incubated at room temperature for 10 min. The mixture was centrifuged at 20,000×g for 40 minutes to separate the pellet, and the supernatant was removed. The pellet was resuspended in PBS (25 μl) and centrifuged again to collect the pellet. All supernatants were combined, and volume was measured. The total S-protein, the pellet fraction of sedimented adjuvant with S-protein, and combined supernatant fraction were quantified using a quantitative S-protein ELISA. The S-protein quantitative ELISA results confirmed that 61.5% (average of two independent protein preparations) of the S-protein was adsorbed onto the liposomes.

### Example 3. In Vivo Toxicity Testing

This example shows analysis of effects on body weight, morbidity, and hyper-inflammatory symptoms in mice treated with compositions prepared as described in Example 1.

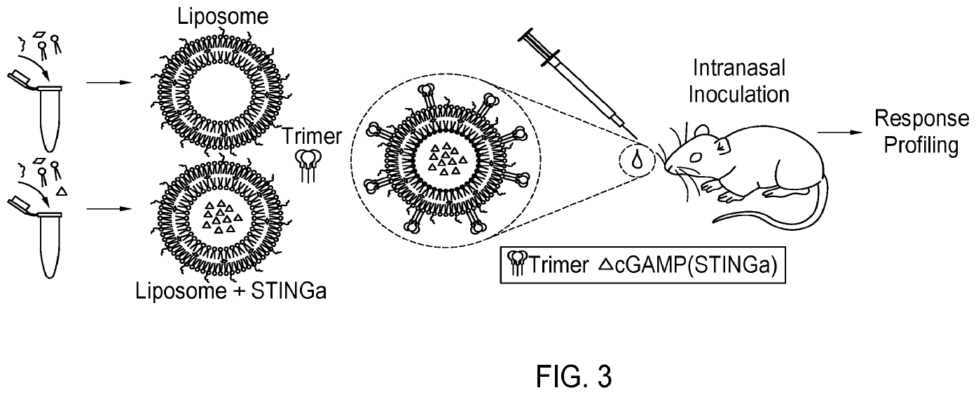

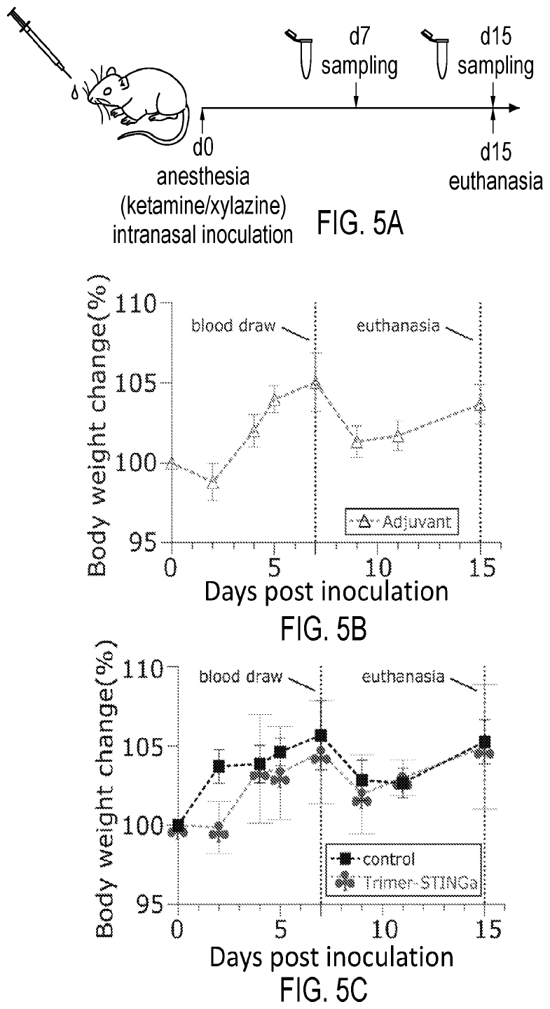

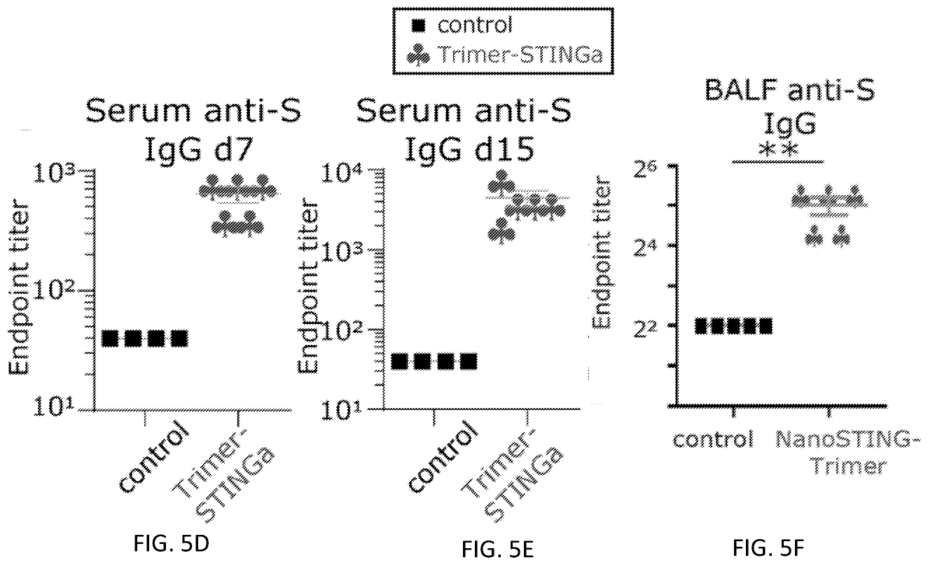

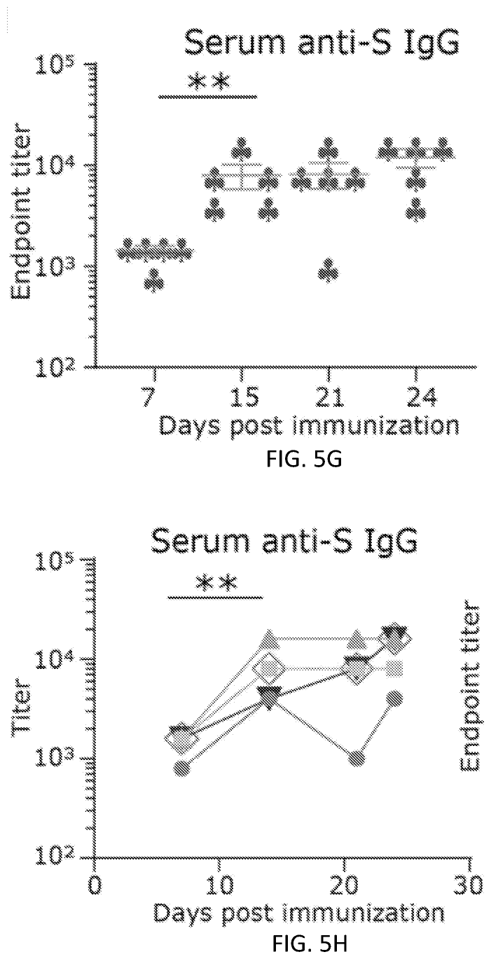

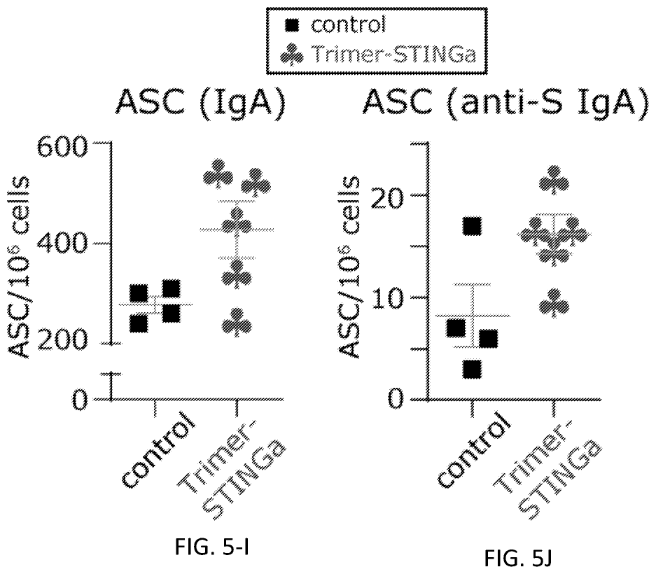

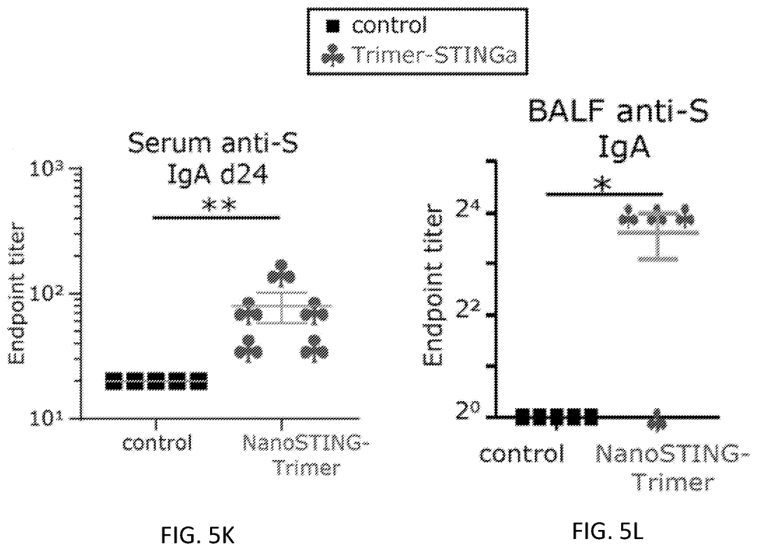

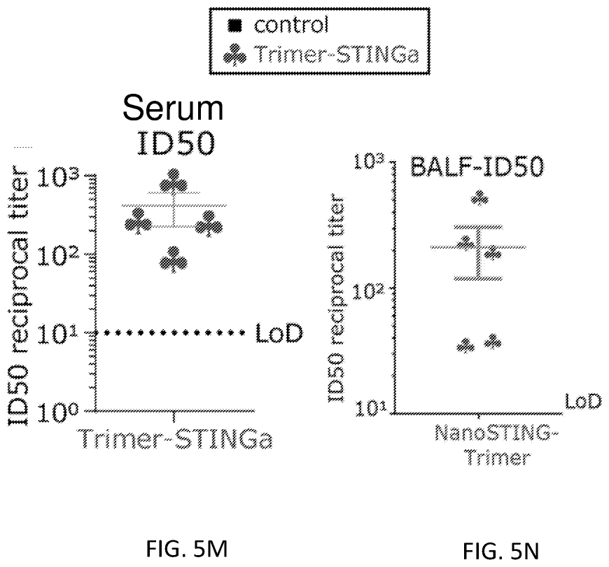

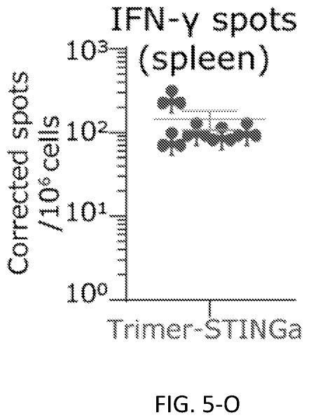

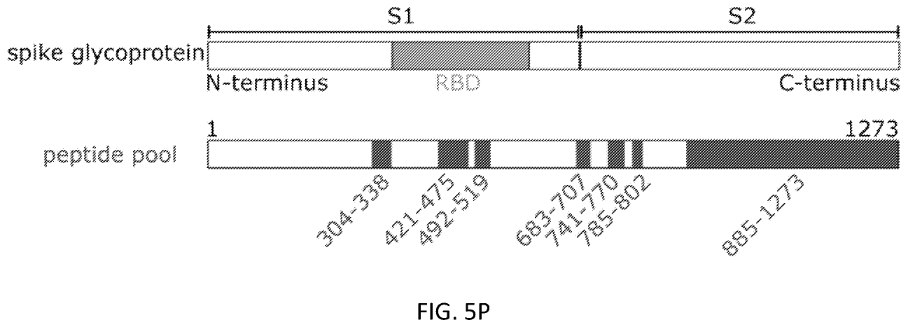

Female, 7-9 week-old BALB/c mice were purchased from Charles River Laboratories. Mice were anesthetized by intraperitoneal injection of ketamine and xylazine before administration of a single dose according to the following scheme: (1) the vehicle only group was administered with 20 μg liposome and modulator without spike protein antigen; (2) the control group was administered 10 μg trimeric spike protein (S-protein); (3) the NanoSTING-Trimer group was administered NanoSTING-Trimer composition comprising 20 μg trimeric spike protein associated with 20 μg of liposome encapsulating STING agonist cGAMP, formulated as described herein; and (4) the Monomer-STINGa group was administered NanoSTING-Monomer composition comprising 4 μg monomeric spike protein associated with 20 μg of liposome encapsulating STING agonist cGAMP, formulated as described herein (see FIG. 3 and FIG. 5A). No weight loss over 14 days was observed for mice administered with compositions comprising only the liposome and modulator without spike protein antigen (FIG. 5B), mice administered with the spike protein antigen alone (FIG. 5C, black boxes), or mice administered with compositions comprising the liposome with modulator and antigen (FIG. 5C, clover leaf data points).

### Example 4. In Vivo Response to Administration of NanoSTING-Trimer Compositions

This example shows in vivo IgG, IgA, and T-cell responses following administration of NanoSTING-Trimer compositions, as described herein.

Two groups of mice were treated by intranasal administration with either a combination of the spike protein trimer antigen adsorbed onto liposomes encapsulating a STING agonist modulator (NanoSTING-Trimer, as prepared according to Example 1) or the spike protein trimer antigen by itself (control), according to the method shown in FIG. 5A. None of the animals showed any clinical symptoms, including loss of weight. Seven days (d7) after immunization, 100% of the mice that received the NanoSTING-Trimer seroconverted and robust anti-S IgG levels with mean dilution titers of 1:640 were detected (FIG. 5D). By day 15 (d15), the serum concentration of the anti-S IgG antibodies increased, and mean dilution titers of 1:4,400 were detected (FIG. 5E). We confirmed that the serum anti-S antibodies were neutralizing with a mean 50% inhibitory dose (ID50) of 1:414 as measured by a GFP-reporter based pseudovirus neutralization assay (SARS-CoV-2, Wuhan-Hu-1 pseudotype) (FIG. 5M).

SARS-CoV-2 spike protein-expressing reporter virus were generated as follows. To determine the titer of neutralizing antibodies in the serum of immunized mice, SARS-CoV-2-S pseudotyped lentiviral system was used as a surrogate for SARS-CoV-2 infection. Pseudotyped viral (PsV) stocks were generated by co-transfecting stable ACE-2 and TMPRSS2 expressing 293 T cells with pLVX-AcGFP1-C1, pMDLg/pRRE, pRSV-Rev, and viral envelope protein expression plasmids pCAGGS containing the SARS-CoV-2, Wuhan-Hu-1 Spike Glycoprotein Gene or VSV-G envelope expressing plasmid pMD2.G to generate the PsV particles. 293T cells stably expressing SARS-CoV-2 receptor human angiotensin-converting enzyme II (ACE2) and plasma membrane-associated type II transmembrane serine protease, TMPRSS2 (293 T/ACE2-TMPRSS2) were cultured in Dulbecco's modified Eagle's medium (DMEM) supplemented with 10% fetal bovine serum. The expression plasmids for SARS-CoV-2 S-protein pCAGGS containing the SARS-CoV-2, Wuhan-Hu-1 Spike Glycoprotein Gene was obtained from BEI Resources (Manassas, Va., USA). Plasmids encoding GFP expressing Lentiviral vector, helper plasmids pMDLg/pRRE, pRSV-Rev, and VSV-G protein-encoding plasmid pMD2.G were obtained from Addgene (Watertown, Mass., USA). The PsV particles in supernatants were harvested 48 hours post-transfection, filtered and stored at −80° C. The dose titer of PsV was determined by infecting ACE-2 and TMPRSS2 expressing 293T cells for 48 h and using a Celigo imaging system for imaging and counting virus-infected fluorescent cells. The viral titers were expressed as fluorescent focus forming units (FFU)/well.

A neutralizing antibody (Nab) titration assay for SARS-CoV-2 was performed as follows. ACE2-TMPRSS2 expressing 293 T cells were cultured overnight in a half area 96-well plate compatible with Nexcelom Celigo imager at a concentration of 1×10⁴ cells per well in 100 μl of complete media. On the day of the assay, heat-inactivated serum from mice was thawed and diluted 1:20 to 1:640 in a six-point, two-fold series in serum-free DMEM. In a 96 well plate, 50 μl of diluted serum was mixed with 50 μl of GFP expressing SARS-CoV-2 spike expressing PsV (150-300 FFU/well) and incubated at 37° C. for 1 hour. After 1 hour, this mixture of added to ACE2-TMPRSS2 expressing 293 T cells and incubated for 48 hours. The infection of target cells was determined by imaging and counting FFU/well using the Celigo imaging system. Each sample was tested in triplicate wells. SARS-CoV-2 Spike 51 rabbit Mab (Clone #007, Sino Biological, Wayne, Pa.) was used as a positive control for neutralizing activity and VSV-G expressing PsV was used as a negative control for the specificity of neutralization function. The 50% inhibitory dose (ID50) titers of NAbs were calculated using nonlinear regression (GraphPad Prism, CA, USA).

Because IgA mediated protection can be a component of mucosal immunity for respiratory pathogens, the IgA responses in the antibody-secreting cells (ASCs) in the spleen at d15 were analyzed by ELISpot assays. IgA-secreting cells were detected using Mouse IgG/IgA Double-Color ELISpot from Immunospot (OH, USA) following the manufacture's instruction. For the total IgA-secreting cells in the spleen, the splenocytes were thawed and seeded to the capture antibody coated ELISPOT plate immediately. The cells were incubated at 37° C. for 16-18 hours, followed by the development. For the anti-S IgA producing cells, thawed splenocytes were cultured in complete media [RPMI-1640 (Corning, N.Y., USA)+10% fetal bovine serum (R&D System, MN, USA), 100 μg/ml Normocin™ (InvivoGen, CA, USA), 2 mM L-Glutamine, 1 mM sodium pyruvate, 10 mM HEPES], and B-Poly-S™ (1:1000 dilution) at 4 million cells/ml. The wells were coated with 10 μg/ml of the spike protein (Sino Biological, PA, USA) overnight at 4° C. The spleen cells were washed and seeded onto the plate at 37° C. for 16-18 hours. FIG. 5-I and FIG. 5J show quantifications of antibody secreting cells (ASC) secreting IgA and S-protein specific IgA, respectively, detected using ELISpot (enzyme-linked immune absorbent spot) assays. The mice treated with NanoSTING-Trimer showed an increase in the number of total IgA secreting (FIG. 5-I) and S-protein-specific IgA secreting (FIG. 5J) ASCs compared to the control group.

Because it is possible that T-cell responses may contribute to protection independent of antibody responses, we evaluated T-cell responses in the treated mice using a pool of 15mers that target highly conserved regions of the S-protein between SARS-CoV and SARS-CoV-2 (FIG. 5P). IFNγ ELISpot assay was performed using Mouse IFN-γ ELISpot Basic kit (ALP), following the manufacturer's instructions (Mabtech, VA, USA). Frozen splenocytes or lung cells were thawed and seeded on the ELISPOT plate (1×10⁵ to 3×10⁵ cells per well) without further culturing. The splenocytes were incubated with the spike protein peptide pool at 1.5 μg/ml/peptide (Miltenyi Biotec, Germany) at 37° C. for 16-18 hours. The ELISpot plates were read using an ImmunoSpot® S6 MICRO analyzer. At d15, all five animals immunized with the NanoSTING-Trimer showed splenic T cell responses with a mean of 144 IFNγ spots/106 cells (FIG. 5-O). Collectively these results show that a single intranasal administration using the NanoSTING-Trimer elicited robust serum neutralizing antibodies and T-cell responses.

These experiments were repeated with the trimeric S-protein to track the kinetics of the immune response over time. We vaccinated groups of mice with either the trimeric version of the S-protein or the adjuvant alone and sampled the sera every seven days for 24 days. Consistent with our previous experiment, we observed early seroconversion with anti-S IgG levels with mean dilution titers of 1:1440. The anti-S IgG responses was 1:8000 at day 14 and changed only slightly at day 24 (1:12,000) (FIG. 5G and FIG. 5H). The IgG concentration in the Bronchoalveolar lavage fluid (BALF) was also elevated in the immunized animals at day 24 (median 1:40) (FIG. 5F).

IgA concentrations were evaluated in the blood and the lung. It was observed that serum anti-S IgA concentrations (dimeric in mice) at mean dilution titers of 1:80 (FIG. 5K). The concentration of IgA was also elevated in the BALF (FIG. 5L). Lastly, it was confirmed that the antibodies in the lung were also neutralizing with a mean 50% inhibitory dose (ID50) of 1:213 (FIG. 5N). Collectively these results established that the NanoSTING-Trimer elicits comprehensive cellular, systemic and mucosal immunity.

### Example 5. Neutralizing Antibody (Nab) Titration Assay for SARS-CoV-2

This example shows experiments investigating local inductive responses within the respiratory tract leading to durable local immunity against inhaled pathogens.

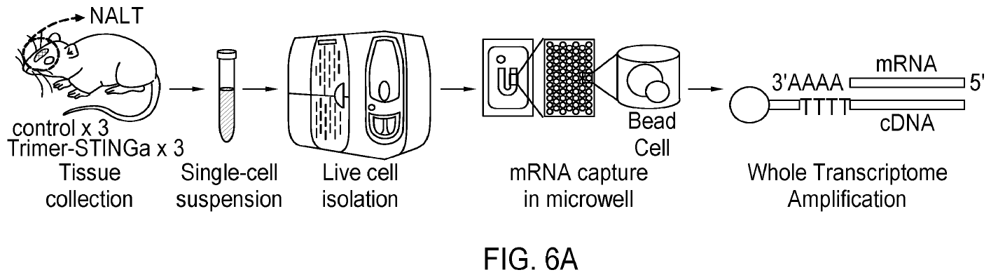

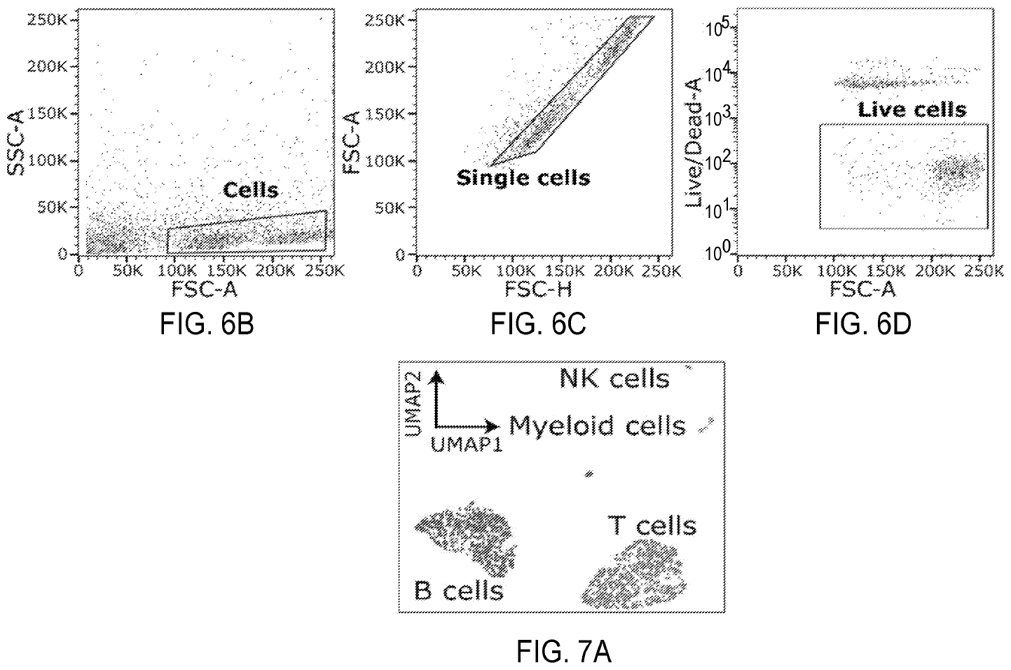

Nasal-associated lymphoid tissue (NALT) was harvested at the time of euthanasia from the animals treated with NanoSTING-Trimer (“Trimer-STINGa”) and animals treated with just the spike protein trimer. Nasal-associated lymphoid tissue (NALT) was isolated from mice after euthanasia, and red blood cells were lysed by incubating the cells in ACK lysis buffer (Thermo Fisher Scientific, Waltham, Mass.) for 3 minutes. NALT samples were converted into single-cell suspensions, sorted into live, single-cell populations using flow cytometry, and subjected to scRNA-seq analysis (FIG. 6A). The single-cell suspensions were subsequently washed with PBS, resuspended in PBS+2% FBS, and exposed to 50 nM Helix NP Blue (BioLegend, San Diego, Calif.) for the detection of live/dead cells.

A BD FACSMelody cell sorter (BD Biosciences, San Jose, Calif.) was used to sort live cells. The flow cytometry gating strategy utilized in this method is shown in FIG. 6B, FIG. 6C, and FIG. 6D. Each group of NALT cells was labeled separately with Sample-Tags from the BD Mouse Immune Single-Cell Multiplexing Kit (BD Biosciences, San Jose, Calif.), according to protocol. Then library preparation was begun with a mixture of ˜6000 cells (3000 cells from each group). Whole transcriptome was prepared using the BD Rhapsody System following the BD “mRNA Whole Transcriptome Analysis (WTA) and Sample Tag Library Preparation Protocol”. The quality and quantity of the final library was assessed by Agilent 4200 TapeStation system using the Agilent High Sensitivity D5000 ScreenTape (Agilent Technologies, Santa Clara, Calif.) and a Qubit Fluorometer using the Qubit dsDNA HS Assay, respectively. The final library was diluted to 3 nM concentration and a HiSeq PE150 sequencer (Illumina, San Diego, Calif.) was used to perform the sequencing. After filtering, a total of 1,398 scRNA-seq profiles were obtained.

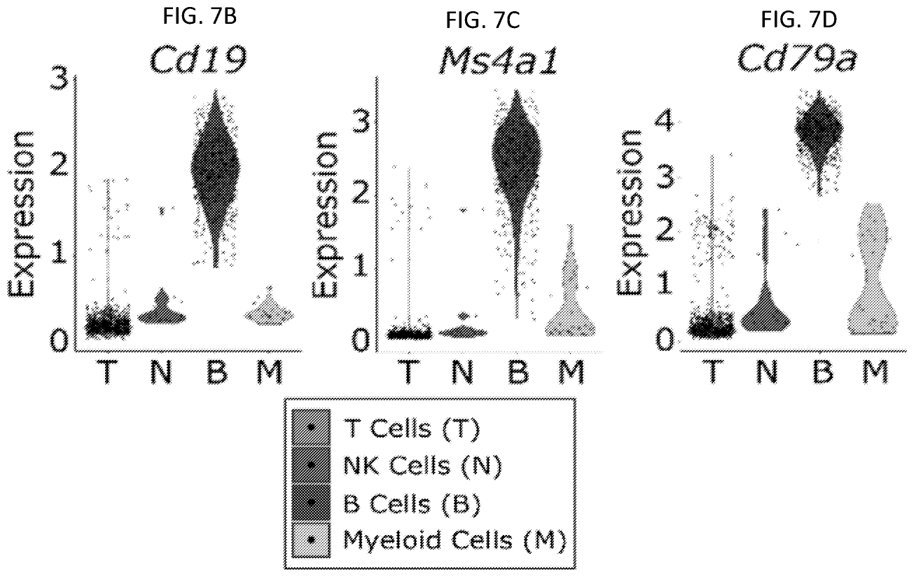

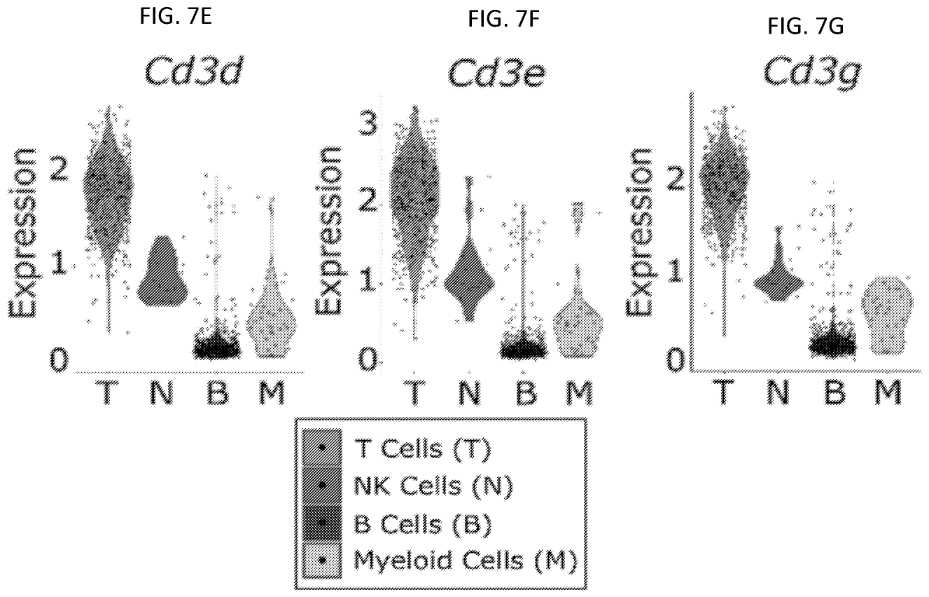

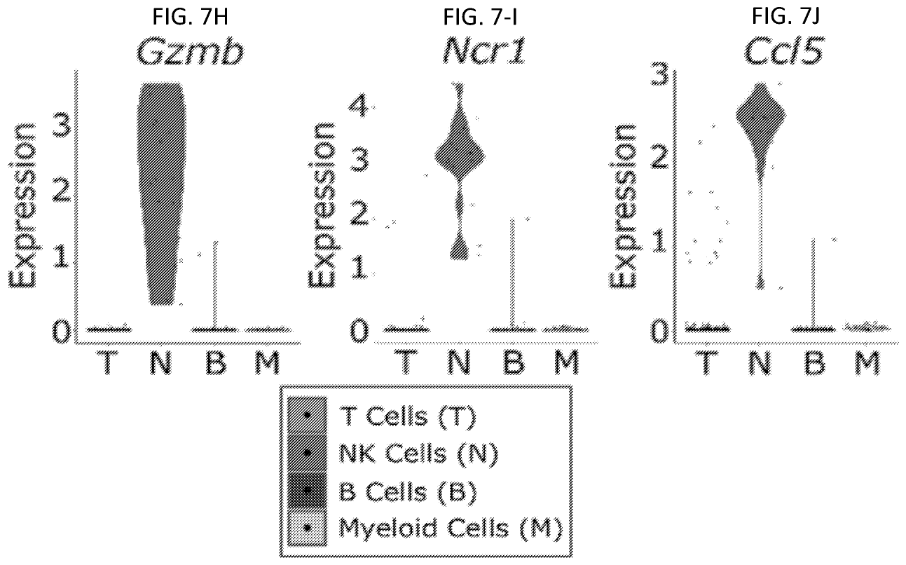

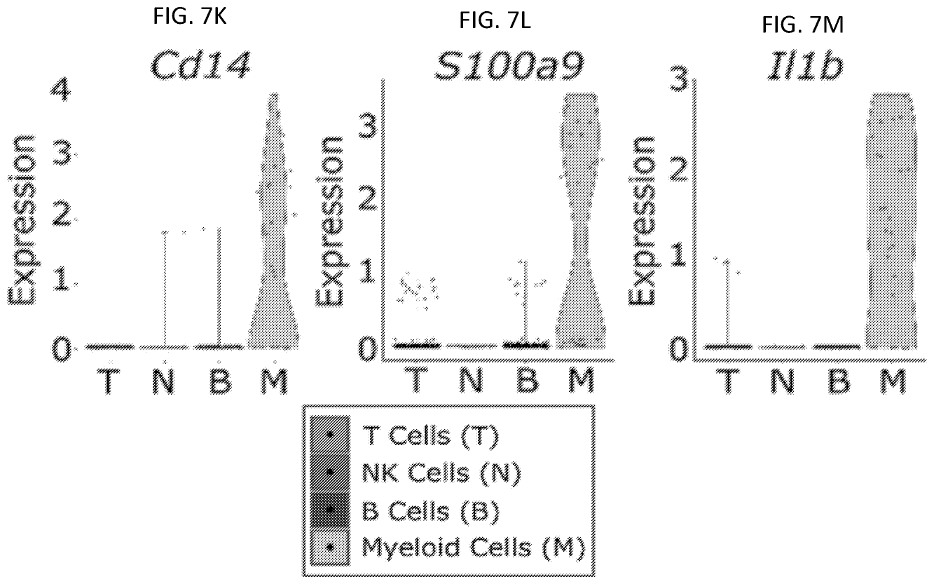

By utilizing uniform manifold approximation and projection (UMAP), B cell, T cell, NK cell and myeloid subpopulations were identified (FIG. 7A). Subpopulations were confirmed using established lineage markers for the B-cell subpopulation (FIG. 7B, FIG. 7C, FIG. 7D), the T-cell subpopulation (FIG. 7E, FIG. 7F, FIG. 7G), the NK-cell subpopulation (FIG. 7H, FIG. 7-I, FIG. 7J), and the myeloid subpopulation (FIG. 7K, FIG. 7L, FIG. 7M). Greater than 95% of the scRNA-seq results in both control and NanoSTING-Trimer groups corresponded to T and B cells. Detailed analyses were performed on these subsets of immune cells.

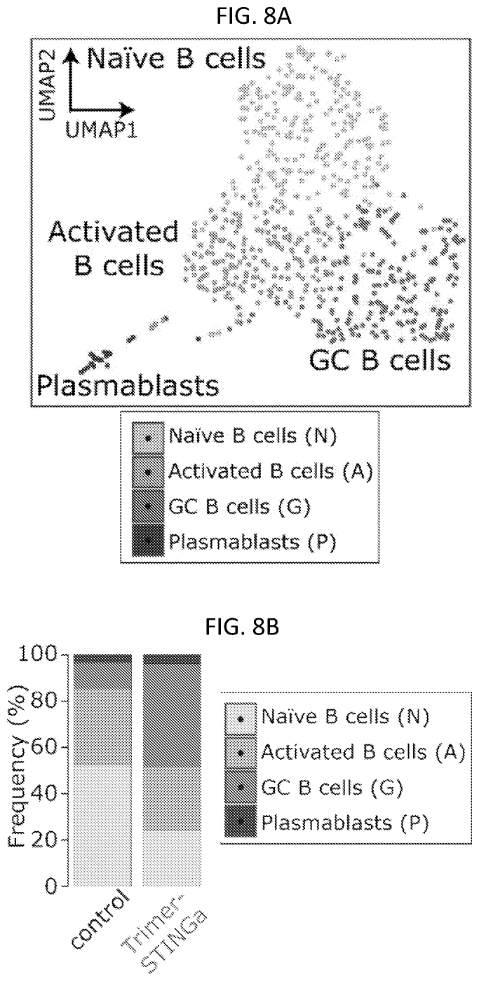

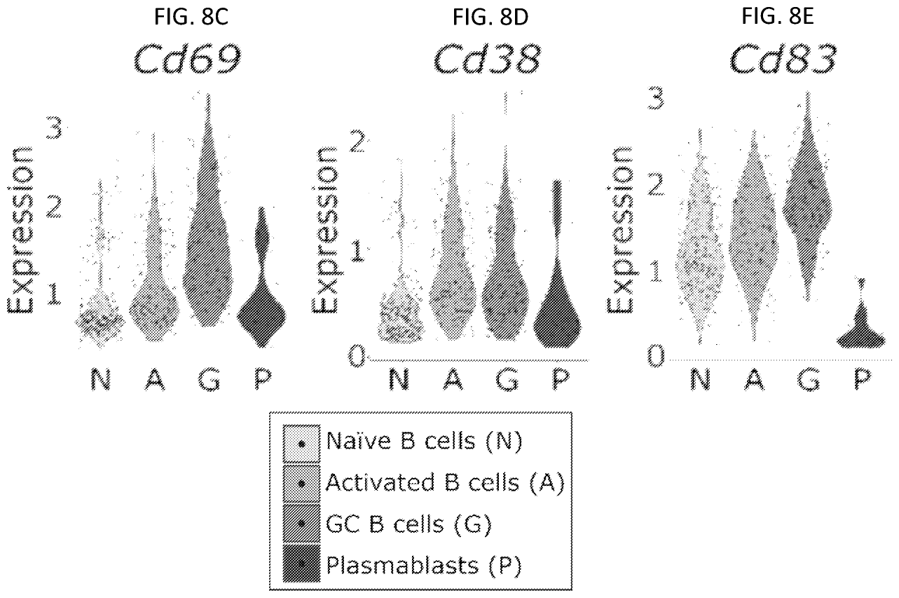

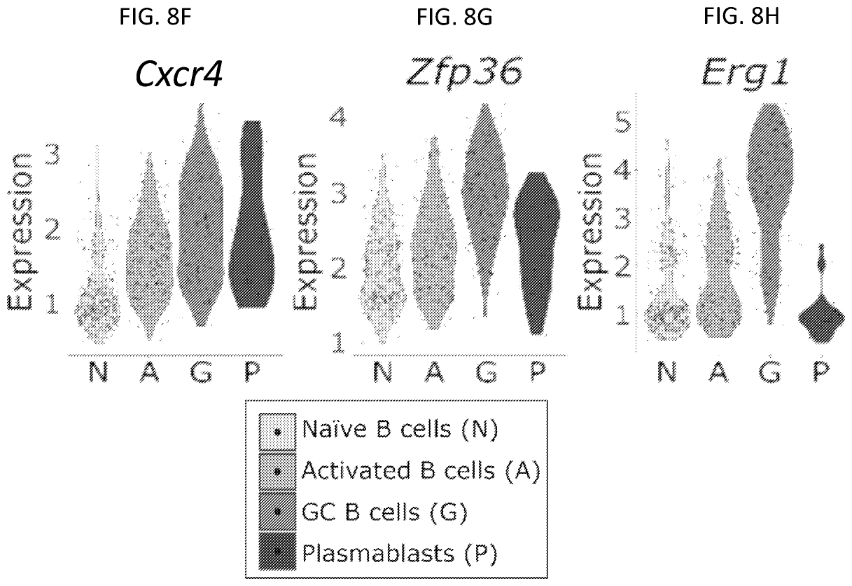

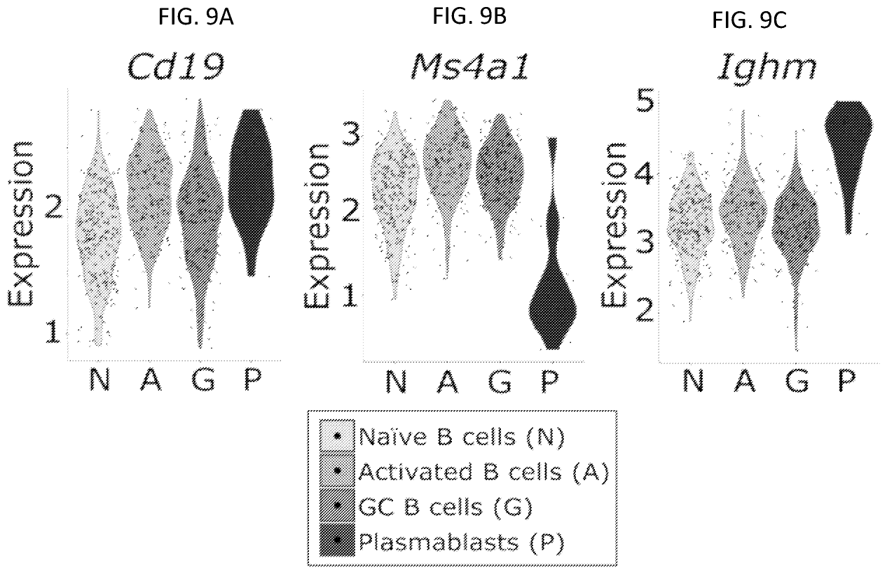

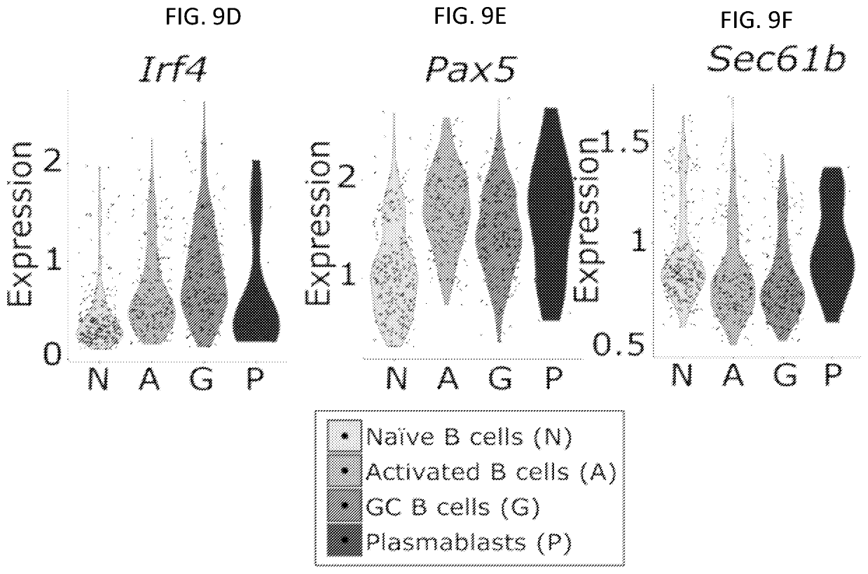

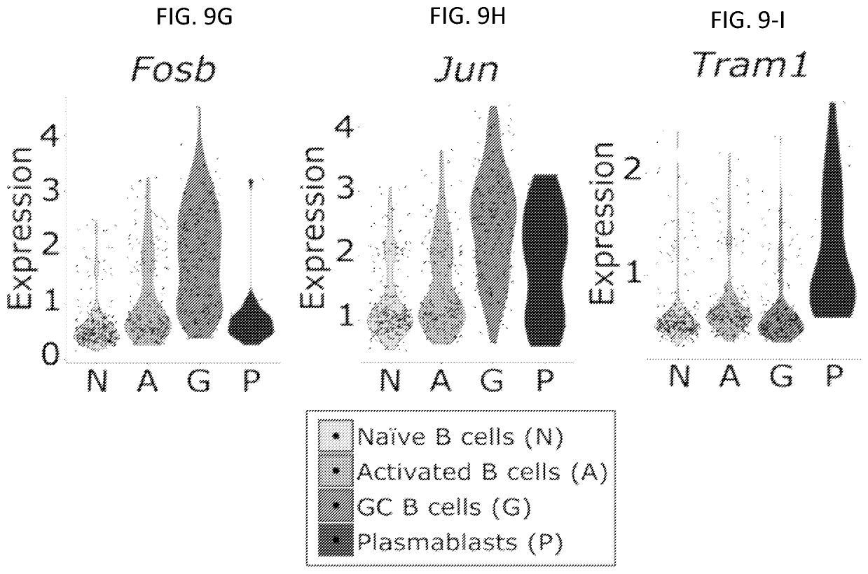

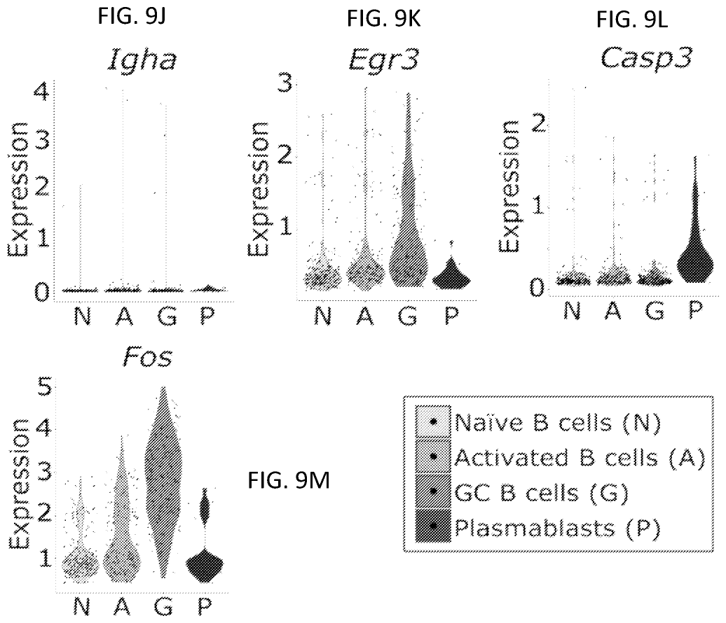

As shown in FIG. 8A, FIG. 8B, and FIGS. 8C-FIG. 9M, four B cell clusters were identified: naïve B cells expressing Cd19 (see FIG. 9A), Ms4a1 (CD20) (see FIG. 9B), Ighm (see FIG. 9C); germinal center B cells (GC B cells) expressing Cd69 (see FIG. 8C), Cd38 (see FIG. 8D), Cd83 (see FIG. 8E), Cxcr4 (see FIG. 8F), Zfp36 (see FIG. 8G), Erg1 (see FIG. 8H), Egr3 (see FIG. 9K), Irf4 (see FIG. 9D), Fosb (see FIG. 9G), Jun (see FIG. 9H), and Fos (see FIG. 9M); an intermediate B cell cluster comprising of activated B cells expressing Cd38 (see FIG. 8D); and ASC (plasmablasts) expressing Sec61b (see FIG. 9F), Casp3 (see FIG. 9L), and Tram1 (see FIG. 9-I) but lacking expression of Ms4a1 (CD20) (see FIG. 9B). Consistent with the role of the NALT as an inductive but not an effector site, we detected only a very small subpopulation of IgA (Igha) expressing cells (see FIG. 9J) at day 15 after administration. Comparisons of the control and NanoSTING-Trimer groups showed a robust increase in the frequency of GC B cells with a concomitant decrease in naïve B cells (FIG. 8B). These results suggested that successful intranasal vaccination promoted differentiation of GC like B cells.

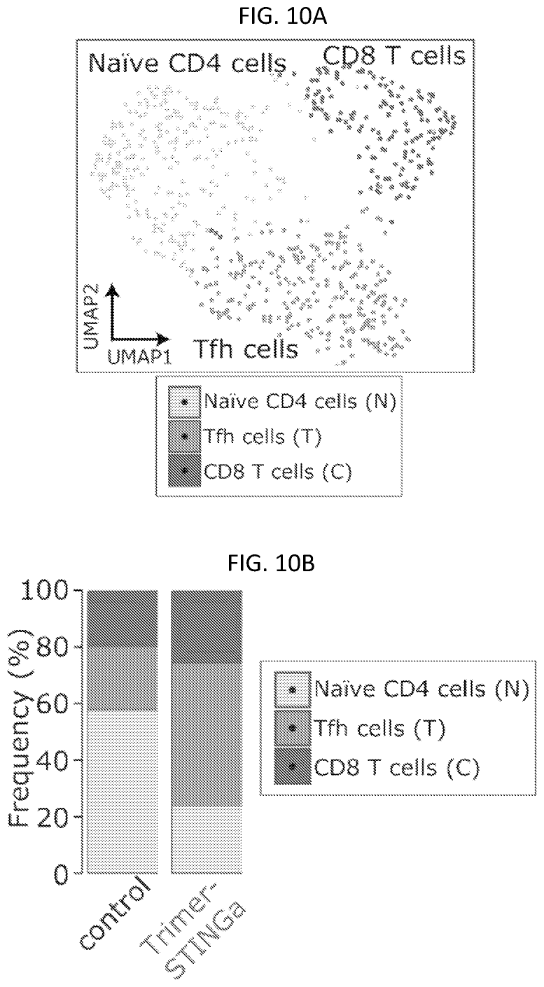

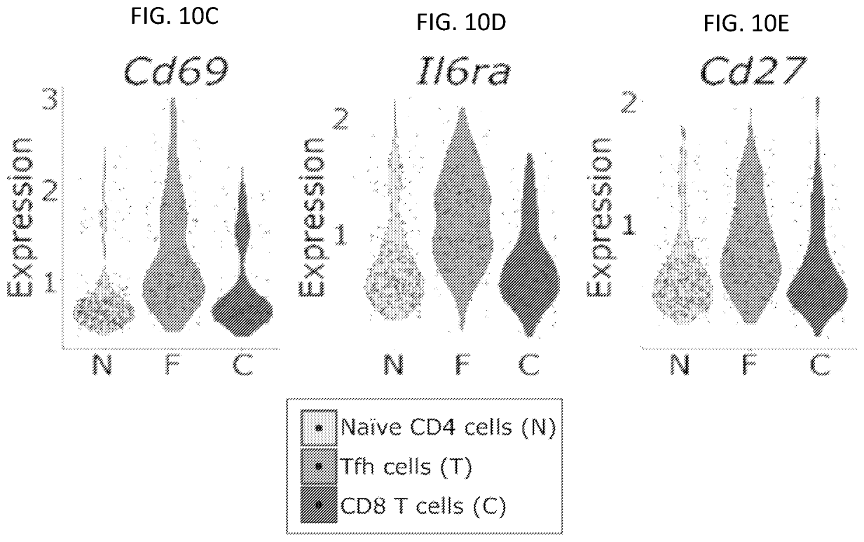

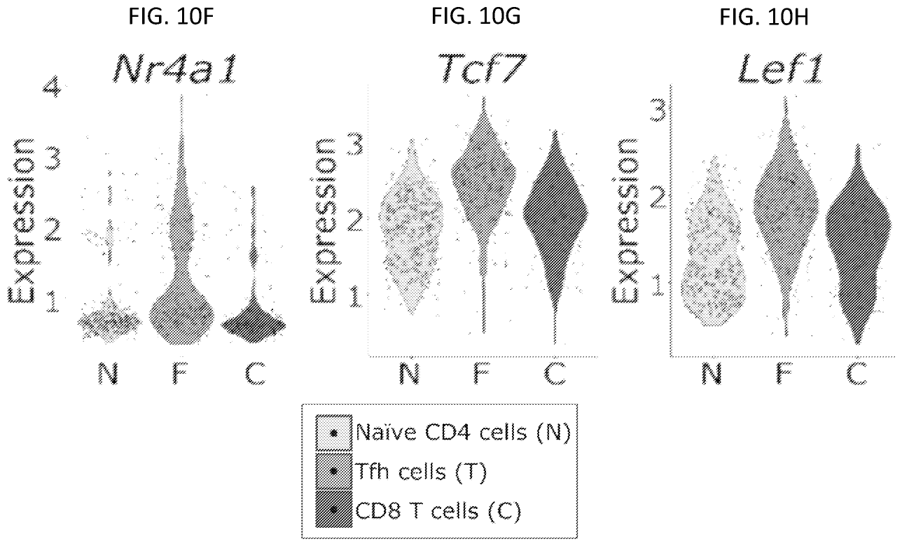

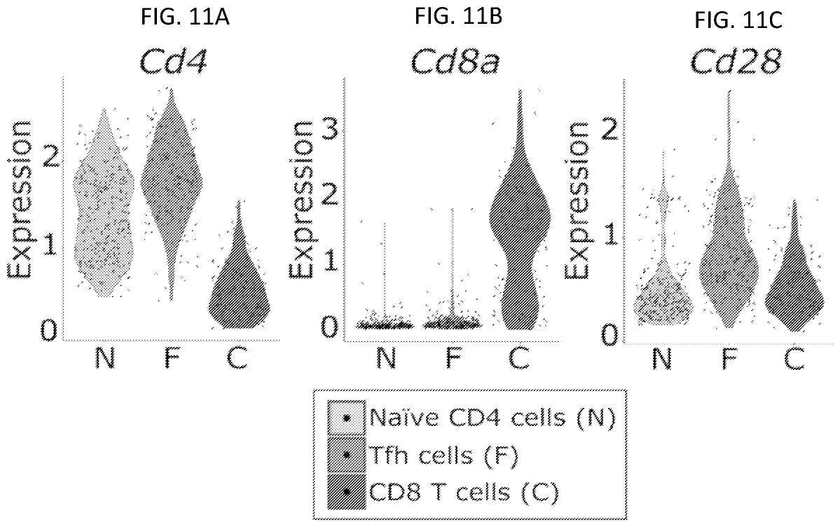

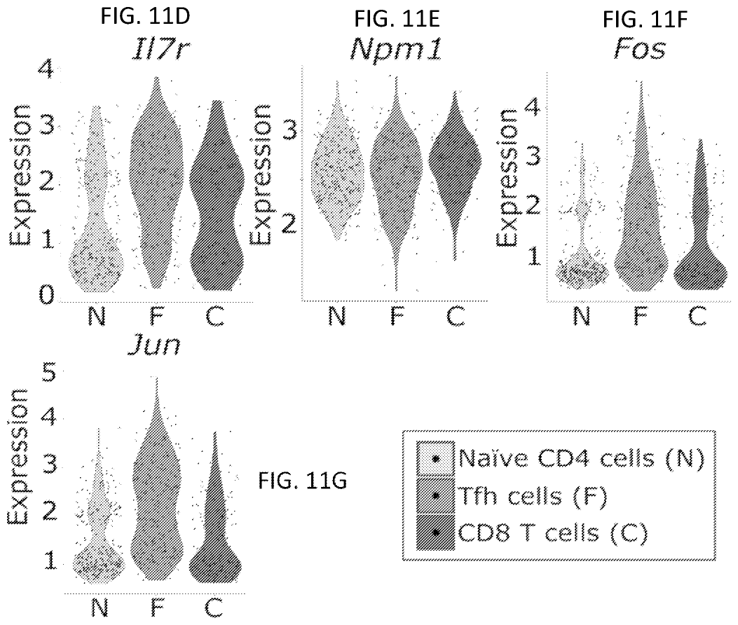

Next, it was investigated if T cells within the NALT supported the B-cell differentiation. We identified three clusters within the T cells: one CD8+ T cell subpopulation expressing Cd8a (see FIG. 11B), and two CD4+ T cell subpopulations (See FIG. 10A). The CD4+ T cells were classified as naïve T cells (naïve) expressing Cd4 (see FIG. 11A) and Npm1 (see FIG. 11E); and T follicular helper like (Tfh) expressing Cd69 (see FIG. 10C), Il6ra (see FIG. 10D), Nr4a1 (Nur77) (see FIG. 10F), Tcf7 (TCF1) (see FIG. 10G), Lef1 (see FIG. 10H), Il7r (see FIG. 11D), Fos (see FIG. 11F), and Jun (see FIG. 11G), and also the memory markers Cd27 (see FIG. 10E) and Cd28 (see FIG. 11C). The prominent difference in the control and the NanoSTING-Trimer groups was an increase in the ratio of Tfh/naïve CD4+ T cells (FIG. 10B).

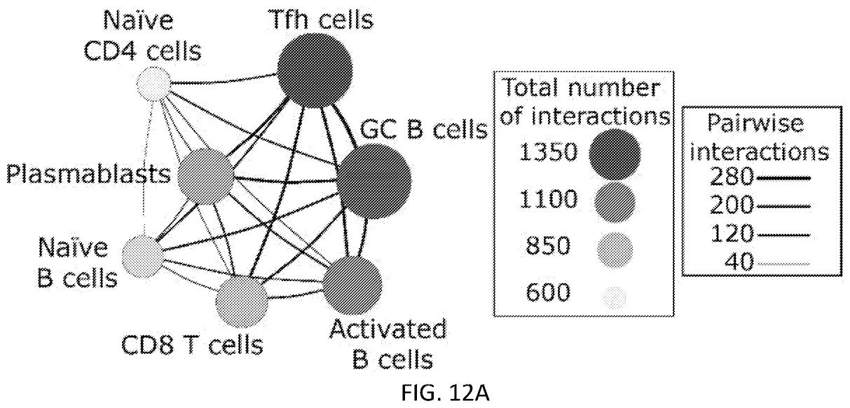

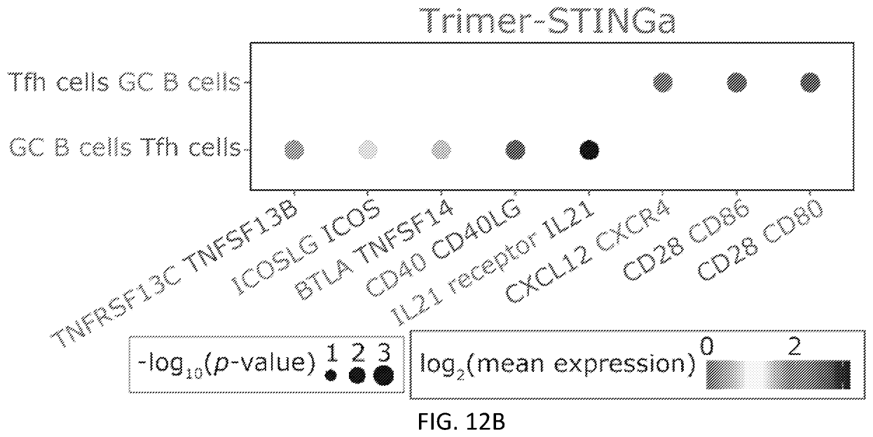

The nature of interactions between these cells was investigated in greater detail since GC B cells and Tfh cells represented the dominant clusters in the NanoSTING-Trimer NALT. First, cell-cell interaction between the different B and T cell clusters in the NALT was visualized. Tfh cells were the dominant interacting cell type and interacted strongly with the GC B cell cluster (FIG. 12A). To investigate interactions at the molecular level, the mice genes were converted to their human counterparts and interacting protein pairs were interrogated using CellPhoneDB. Briefly, T cells and B cells were categorized by their subpopulations and group (treated or Trimer-STINGa) to 14 cell types. According to statistical tests calculated CellPhoneDB, the ligand-receptor pairs with p values >0.05 were filtered out, and the relationship between different cell types with the significant pairs was evaluated. Several well-documented receptor-ligand pairs known to promote interaction between GC B cells and Tfh cells, BAFFR (TNFRSF13C)-BAFF(TNFSF13B); ICOSLG-ICOS; CD40-CD40L; and IL21-IL21R were detected reciprocally on the B cells and the Tfh cells within the NALT (FIG. 12B). These results showed that upon immunization with the NanoSTING-Trimer, the NALT promoted a GC-like T-cell dependent activation and differentiation of B cells, which in turn can promote long-lasting immunity in the respiratory compartment.

These results show that NanoSTING compositions comprising antigens, which can comprise an intranasal subunit vaccine, can induce systemic neutralizing antibodies, mucosal IgA responses in the lung and nasal compartments, and Th1 T-cell responses in the lung of mice. Single-cell RNA-sequencing confirms the concomitant activation of T and B cell responses in germinal center like manner within the nasal-associated lymphoid tissues (NALT), and confirms its role as an inductive site for immunity. The ability to elicit immunity in the respiratory tract has the potential to prevent initial establishment of infection in individuals and to prevent disease transmission across humans.

### Example 6. Responses in the Lung and Nasal Compartments to NanoSTING-S Administration

This example shows T-cell responses in the lung compartment and IgA responses in the nasal compartment in response to in vivo administration of NanoSTING compositions disclosed herein.

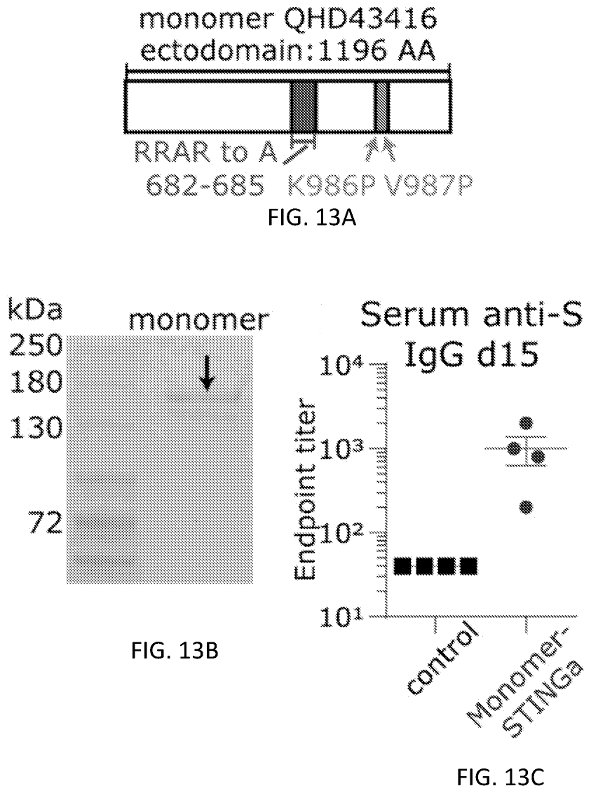

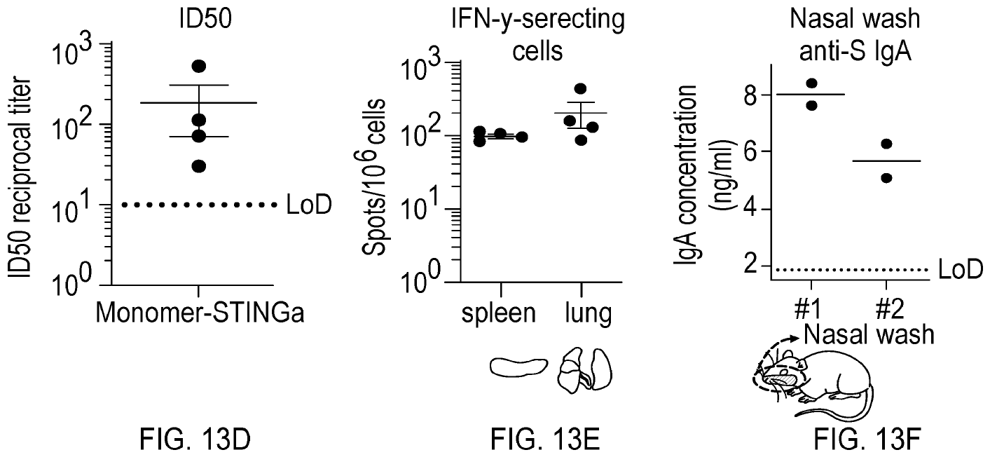

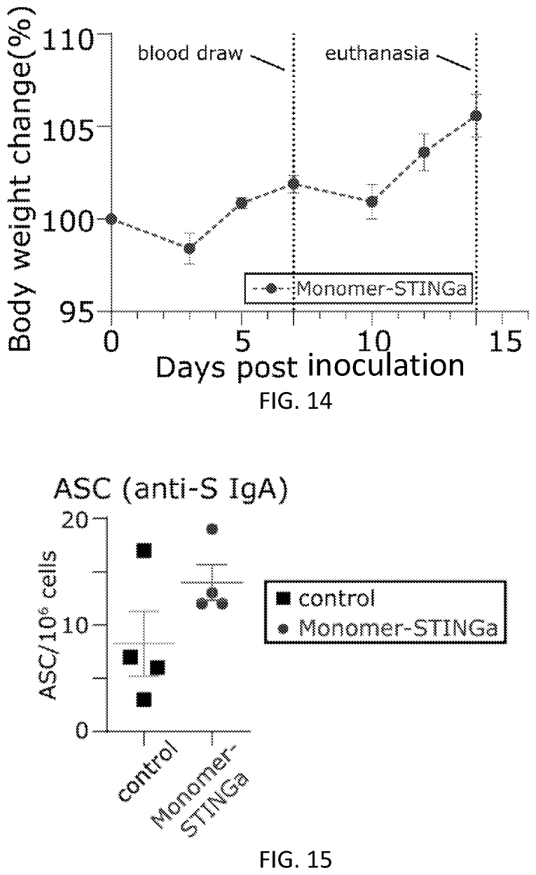

To investigate whether the monomeric spike protein (S-protein) could also elicit a comprehensive immune response, a monomeric version of the S-protein containing mutations to the Furin binding site and a pair of stabilizing mutations (Lys986Pro and Val987Pro) was used (FIG. 13A). SDS-PAGE of the monomeric protein showed a band between 130-180 kDa (FIG. 13B). Each of four mice were treated with the monomeric spike protein (S-protein) or a NanoSTING-Monomer composition comprising a monomeric spike protein associated with a liposome encapsulating the STING agonist cGAMP. NanoSTING monomer compositions were formulated using the same protocol as that described in Example 1, using a monomeric spike protein instead of a trimeric spike protein. No weight loss was observed in any of the animals (FIG. 14). At d15, 100% of the animals seroconverted, and the mean serum concentration of the anti-S-protein (“Anti-S”) IgG antibodies was 1:1000 (FIG. 13C). We confirmed that the serum anti-S-protein (“anti-S”) antibodies were neutralizing with a mean ID50 of 1:188 (FIG. 13D).

The antibody responses in the nasal compartment, and total IgA in the nasal wash from two animals was measured by ELISA. Both of the nasal washes also had detectable anti-S IgA antibodies at a mean concentration of 7 ng/ml (FIG. 13F). Consistent with the ability of the Monomer-STINGa to elicit mucosal immune responses, we also confirmed the detection of 5-specific IgA secreting ASCs in the spleen of these mice (FIG. 15). These results established that vaccination with the Monomer-STINGa elicits systemic immunity, T-cell responses in the lung and spleen, and mucosal IgA responses.

Animal models have shown that T cells in the lung are necessary for protection against pulmonary infection by respiratory pathogens. Accordingly, we evaluated S-protein specific T-cell responses in the lung of the vaccinated animals with the conserved pool of peptides (FIG. 5P). T-cell responses were detected in the spleen at a mean of 100 IFNγ spots/10⁶ cells, and in the lung on day 15 following treatment at a mean of 206 IFNγ spots/106 cells (FIG. 13E).

Collectively, these results established that intranasal administration using the NanoSTING-monomer also elicited robust serum neutralizing antibodies and T-cell responses in both the lung and the spleen.

### Example 7: Preparation and Characterization of NanoSTING-N Composition

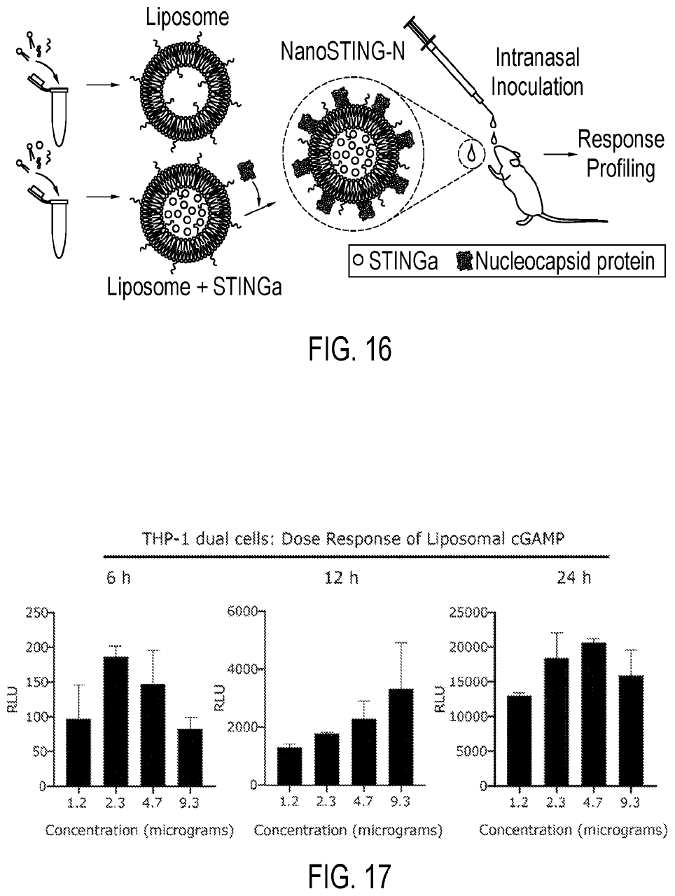

This example describes the preparation and characterization of lipid-based particles, comprising a STING agonist modulator and a nucleocapsid antigen, useful in compositions for treating or preventing a disease (e.g., a respiratory disease) in a subject. To facilitate efficient priming of the immune system within the respiratory compartment, the STING agonist was encapsulated within negatively charged liposomes to yield compositions comprising a liposome, a modulator, and a nucleocapsid antigen associated with the surface of the liposome (“NanoSTING-N”) (see, FIG. 16).

The liposomes were composed of a molar ratio of 10:1:1:1 of DPPC, DPPG, cholesterol, and DPPE-PEG2000. 1,2-dipalmitoyl-sn-glycero-3-phosphocholine (DPPC), 1,2-dipalmitoyl-sn-glycero-3-phospho-(1′-rac-glycerol) (DPPG), and 1,2-dipalmitoyl-sn-glycero-3-phosphoethanolamine-N-[methoxy(polyethylene glycol)-2000] (DPPE-PEG2000) were obtained from Avanti Polar Lipids (Alabaster, Ala.). Cholesterol was obtained from Sigma Aldrich (St. Louis, Mo.). To prepare the liposomes, the lipids were mixed in chloroform (CHCl₃) and methanol (CH₃OH), and the solution was evaporated by a vacuum rotary evaporator for approximately 80 minutes (min) at 45° C. The resulting lipid thin films were dried until all organic solvent was evaporated. The lipid film was hydrated by adding a pre-warmed cGAMP solution (0.3 mg/ml cGAMP in PBS buffer at pH 7.4). The STING agonist, 2′-3″cyclic guanosine monophosphate adenosine monophosphate (cGAMP) was obtained from Chemietek (Indianapolis, Ind.).

The hydrated lipids were mixed at elevated temperature 65° C. for an additional 30 min, then subjected to freeze-thaw cycles. The mixture was sonicated at 40 kHz for 60 min using a Branson Sonicator. SARS-CoV-2 (2019-nCoV) nucleocapsid recombinant protein (N-protein) was purchased from BEI Resources (VA, USA #NR-53797) and tested at two different doses (10 μg and 20 μg) in combination with the STING-liposomal suspensions. Recombinant N-protein was mixed with NanoSTING liposomes encapsulating cGAMP to allow the adsorption of the protein on the liposomes. The free untrapped cGAMP was removed by Amicon Ultrafiltration units using a molecular weight cut off of 10 kDa. The cGAMP-liposomes were washed three times using PBS buffer. The cGAMP concentration in the filtrates was measured by Take3 Micro-Volume absorbance analyzer of Cytation 5 (BioTek) against a calibration curve of cGAMP at 260 nm (FIG. 36A). The final concentration of liposomal encapsulated cGAMP and encapsulation efficiency were calculated by subtracting the concentration of free drug in the filtrate (standard curve shown in FIG. 36B). NanoSTING-N compositions were kept at room temperature to allow the adsorption of the protein onto the liposomes. The formulated NanoSTING-N composition was stored at 4° C. and used for up to 2 months. These results suggested that formulated N-protein STING-liposomes vaccine exists in a nanoparticulate colloidal form.

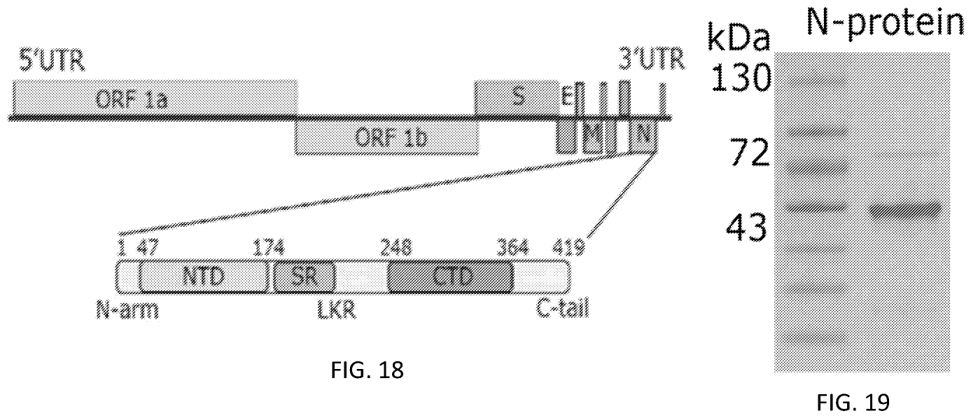

FIG. 18 shows a schematic representation of SARS-CoV-2 N-protein, showing it to be comprised of a nucleic acid (RNA) binding domain, a C-terminal dimerization domain and three intrinsically disordered domains that can promote phase separation with nucleic acids (FIG. 18). Insect-cell derived recombinant N-protein was used as the immunogen and SDS-PAGE confirmed a dominant band with a size of 47 kD (FIG. 19).

### Example 8: Effects of NanoSTING Composition on Interferon Responses In Vitro

This example shows experimental confirmation that NanoSTING induces interferon regulatory factor (IRF) responses using the THP-1 monocytic cells that stably express secreted luciferase downstream of an IRF responsive promoter.

THP-1 DUAL cell line cells (Invivogen) were cultured in a humidified incubator at 37° C. and 5% CO2 and grown in RPMI/10% FCS (Corning, N.Y., USA). The THP-1 DUAL cell line cultures were supplemented with the respective selection agents (100 μg/ml zeocin+10 μg/ml blasticidin) and the corresponding selection cytostatics from Invivogen.

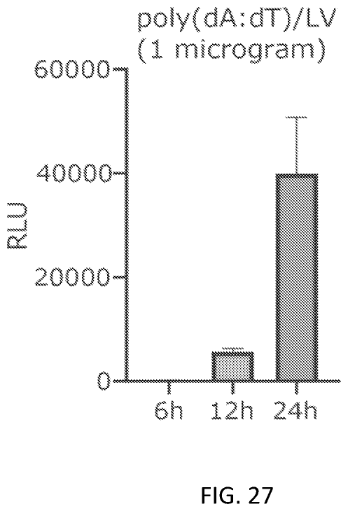

THP-1 DUAL cells were cultured in 96 well plate at 1×10⁵ cells/well in 180 μl growth media at the outset of cell stimulation experiments comprising luciferase reporter enzyme detection. Serial dilutions of NanoSTING-N were made in growth medium before cells were incubated at 37° C. for 24 h in the presence of NanoSTING-N at concentrations of 1.2 μg (micrograms), 2.3 μg, 4.7 μg, and 9.3 μg. For detection of IRF activity, 10 μl of culture supernatant was collected per well at time points of 6 h, 12 h, and 24 h and transferred to a white (opaque) 96 well plate. 50 μl QUANTI-Luc™ (Invivogen) substrate solution was added per well followed immediately by luminescence measurement (in relative light units (RLU)) using a Cytation 7 instrument (Cytation 7, Bio-Tek Instruments, Inc.). FIG. 27 shows a quantification of interferon response factor (IRF) responses in THP-1 dual cells in response to type I interferon inducer poly(dA:dT) complexed with cationic lipid-based transfection reagent LyoVec™ (InvivoGen) (“poly(dA:dT)/LV”) at 6 hours, 12 hours, and 24 hours after exposure to 1 μg poly(dA:dT)/LV.

THP-1 dual cells were contacted and performed kinetic measurements for 24 hours by measuring the luciferase activity in the supernatant (FIG. 27). THP-1 secretion of luciferase was observed only at low levels at 6 h, increasing more than an order of magnitude at all concentrations of NanoSTING-N at 12 hours, and reaching highest observed levels at 24 h (FIG. 17). At 24 hours, all concentrations of NanoSTING-N showed similar THP-1 luciferase secretion.

### Example 9: In Vitro Nucleocapsid DNA Binding Assay

This example shows DNA binding studies performed to determine N-protein nucleocapsid capacity for functional association with plasmid DNA.

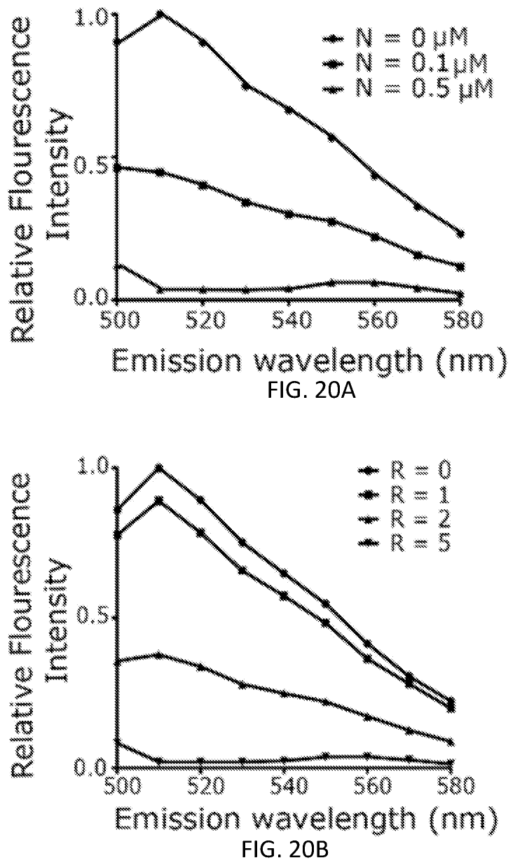

Branched-chain PEI was used as a positive control (Sigma Chemical Co., St. Louis, Mo. #408727). DiYO-1 (AAT Biorequest #17579) and Plasmid (pMB57.6)-DNA complexes were mixed in equal volumes of DNA and DiYO-1, a bis-intercalating fluorophore whose quantum yield increases by several orders of magnitude upon binding to ds-DNA, (in 20 mM HEPES, 100 mM NaCl, pH=7.4) to achieve a final concentration of 400 nM and 8 nM, respectively. The solution was left at RT for 5 h before use. Next, PEI was added at different concentrations (R=0, 1, 2, 5 where R is the molar ratio of PEI Nitrogen to DNA phosphate) to DNA-DiYO-1 solution, vortexed for 1 minute left for 2 h to equilibrate. We measured the fluorescence intensity of the solution at excitation and emission wavelength of 470 nm and 510 nm, respectively (FIG. 20B). The procedure was repeated with SARS-CoV2 nucleocapsid (N-protein) instead of PEI (FIG. 20A). In these experiments, the nucleocapsid (N-protein) was added at concentrations of 0.1 and 0.5 μM to the DNA-DiYO-1 solution.

The N-protein was able to quench the fluorescence of the DNA-DiYO-1 complex in a concentration-dependent manner (FIG. 20A). At a concentration of 0.5 μM the N-protein decreased the fluorescence intensity to the same extent (e.g., 96.3%) as the known synthetic polycation DNA condensation agent, polyethylenimine (PEI) R=5 (FIG. 20B). These results confirmed that the recombinant N-protein is potent to binder of dsDNA.

### Example 10: Induction of Humoral Responses Using NanoSTING-N Compositions

This example shows in vivo testing of NanoSTING-N composition effects on humoral responses in mice.

Female, 7-9-week-old BALB/c mice were purchased from Charles River Laboratories. Before administration of compositions, mice were anesthetized by intraperitoneal injection of ketamine and xylazine. Animals were treated via a single intranasal administration with one of two different concentrations of the NanoSTING-N composition comprising 20 μg of the liposome-STING adjuvant for immunization and either 10 μg or 20 μg nucleocapsid protein (NanoSTING-N10 and NanoSTING-N20, respectively).

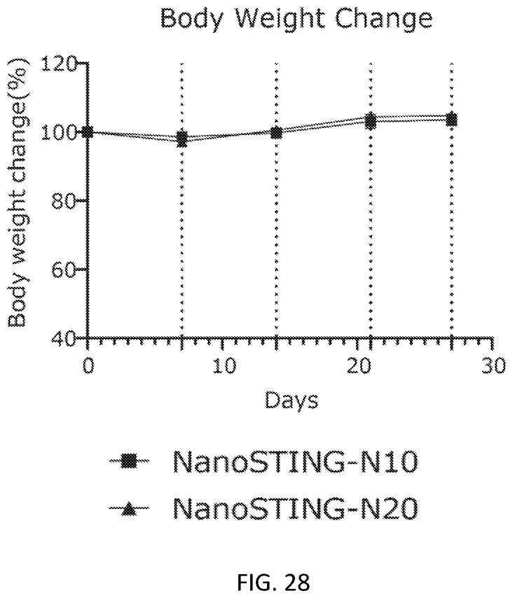

Bodyweight of the animals was measured every 7 days for four weeks after administration of NanoSTING-N composition. No animals treated with NanoSTING-N10 or NanoSTING-N20 were observed to undergo weight loss or gross abnormalities over 27 days (FIG. 28).

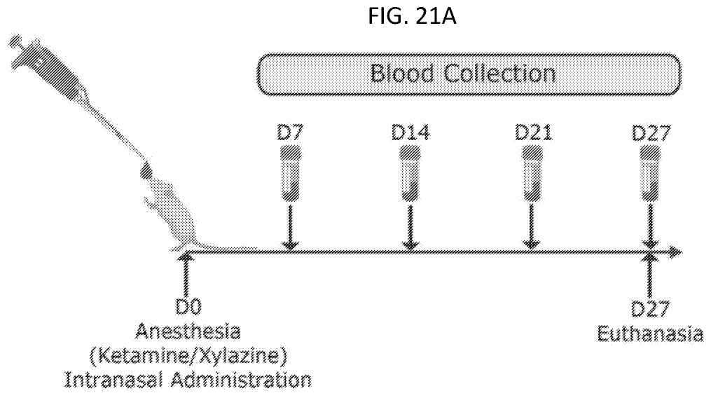

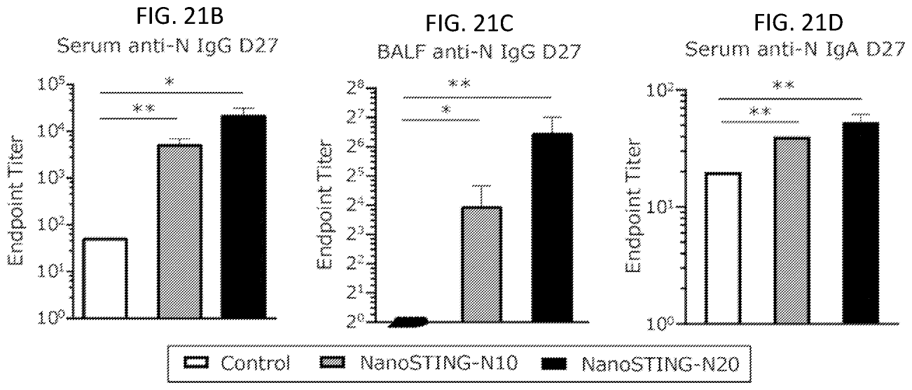

Sera was collected from the subjects at the 7th, 14th, 21st, and 27th days post-administration for detection of the humoral immune response (FIG. 21A). Blood was maintained at room temperature for 10 minutes to facilitate clotting before serum samples were centrifuged for 5 minutes at 2000 g. Serum was collected from the centrifuged samples and stored it at −80° C. and was later used for ELISA assays. Bronchoalveolar lavage fluid (BALF), lung, and spleen were harvested from the subjects at 27 days after the intranasal administration. Sera and other collected biological samples were maintained at −80° C. in the presence of protease inhibitors for long-term storage. After dissociation, the splenocytes and lung lymphocytes were frozen in FBS+10% DMSO and stored in the liquid nitrogen vapor phase until further use.

### Example 11: Induction of Humoral Responses Using NanoSTING-N Compositions

This example shows nucleocapsid-specific IgG and IgA responses induced in subjects by treatment with NanoSTING-N compositions.

Anti-N-protein antibody titers were determined in serum or other biological fluids using ELISA. Briefly, 1 μg/ml N-protein (Sino Biological, PA, USA) was coated onto ELISA plates (Corning, N.Y., USA) in PBS overnight at 4° C. for 2 hours at 37° C. The plate was then blocked with PBS+1% BSA (Fisher Scientific, PA, USA)+0.1% Tween20™ (Sigma-Aldrich, MD, USA) for 2 hours at RT. After washing, we added the samples at different dilutions. We detected the captured antibodies by HRP-conjugated anti-mouse IgG (Jackson ImmunoResearch Laboratories, 1 in 5,000; PA, USA), anti-mouse IgA (Bethyl Laboratories, 1:10,000; TX, USA), and detection antibody against mouse IgA (1:250) from the mouse total IgA ELISA kit from Invitrogen (CA, USA). We obtained the positive control (anti-N IgG) from Abeomics (CA, USA).

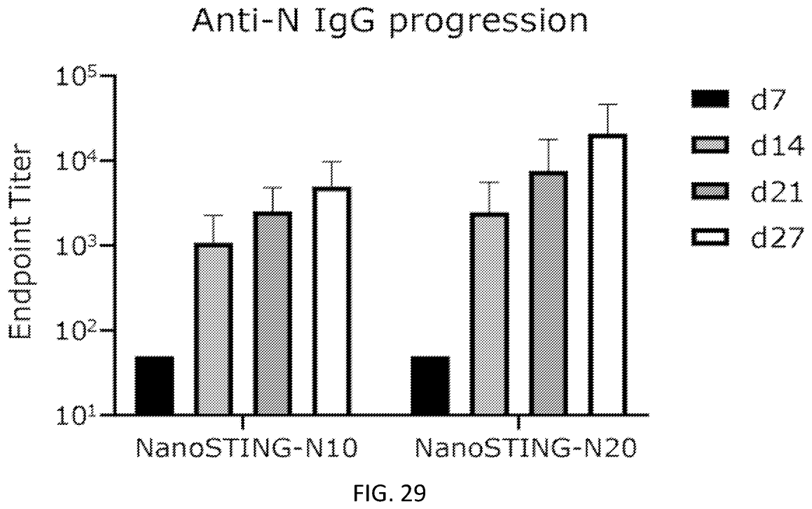

Fourteen days (D14) after immunization, 100% of the mice that received the NanoSTING-N seroconverted and robust anti-N IgG levels with mean dilution titers of 1:640 were detected (FIG. 29). By day 27 (D27) after treatment, the serum concentration of the anti-N IgG antibodies increased in serum samples, and mean dilution titers of 1:4,400 were detected (FIG. 21B). The IgG responses at both doses was similar at all of the timepoints tested and although the IgG titers elicited by the NanoSTING-N20 were higher than NanoSTING-N10, the difference was not significant (FIG. 21B). In contrast to vaccination with the trimeric NanoSTING-S (early response at day 7), the kinetics of IgG responses were delayed, and responses were only observed at day 14. We also assessed the SARS-CoV-2-specific antibody responses in bronchoalveolar lavage fluid (BALF) of immunized mice. The BALF samples from NanoSTING-N10 and NanoSTING-N20 showed mean IgG titers of 1:15 and 1:86 respectively (FIG. 21C).

IgA-mediated protection is an essential component of mucosal immunity for respiratory pathogens. IgA responses in the serum were tested. NanoSTING-N10 vaccinated mice yielded serum IgA levels with mean dilution titers of 1:40 and Nano-STING-N20 vaccinated animals yielded serum IgA levels with mean dilution titers of 1:53 (FIG. 21D). Collectively these results established that immunization with the N-protein yielded robust humoral immunity in the serum and lung.

### Example 12: Processing of Spleen and Lungs for ELISPOT and Flow Cytometry

This example illustrates methods for harvesting and processing spleen and lung tissue from subjects treated with NanoSTING compositions.

For isolation of lung lymphocytes, the lung vasculature was perfused with 5 ml of 1 mM EDTA in PBS without Ca²⁺ or Mg²⁺ injected into the right cardiac ventricle. Each lung was cut into 100-300 mm² pieces using a scalpel. Minced tissue was transferred to a tube containing 5 ml of Digestion Buffer containing Collagenase D (2 mg/ml, Roche #11088858001) and DNase (0.125 mg/ml, Sigma #DN25) in 5 ml of RPMI for 1 h and 30 minutes at 37° C. in a water bath with vortexing performed after every 10 min. The remaining intact tissue was disrupted by passage (6-8 times) through a 21-gauge needle. Cells were incubated for 90 min and 500 μl of ice cold stopping Buffer (1×PBS, 0.1M EDTA) added to stop the reaction. Tissue fragments and dead cells were removed using a 40 μm disposable cell strainer (Falcon), and cells were collected after centrifugation. Red blood cells were lysed by resuspending the cell pellet in 3 ml of ACK Lysing Buffer (Invitrogen) and incubated for 3 minutes at room temperature (RT), followed by centrifugation. Supernatants were discarded and cell pellets were resuspended in 5 ml of complete RPMI medium (Corning, N.Y., USA). Next, the spleens were collected in RPMI medium and homogenized through a 40 μm cell strainer using the hard end of a syringe plunger. After that, splenocytes were incubated in 3 ml of ACK lysis buffer for 3 minutes at RT to remove red blood cells (RBCs), then passed through a 40 μm strainer to obtain a single-cell suspension. lung lymphocytes and splenocytes were counted by the trypan blue exclusion method.

### Example 13: Effects of SARS-CoV-2 Nucleocapsid Protein on T-Cell Responses In Vivo

This example shows evaluation of effects of intranasal administration of SARS-CoV-2 nucleocapsid protein on systemic T cell responses.

One of the advantages of administering N-protein based compositions disclosed herein (e.g., NanoSTING-N compositions) to a subject is the ability to elicit T cell responses. A Th1/Tc1 polarized response (e.g., comprising IFN-γ secretion) can be indicative of protective responses whereas a Th2 polarized response (e.g., comprising IL-4 secretion) can have the potential to cause antibody dependent enhancement (ADE) for viral infection. To assess the vaccine-induced N-specific T cell responses, splenocytes and lung lymphocytes were harvested and processed as described in Example 12 from subjects 27 days after treatment with SARS-CoV-2 nucleocapsid protein (N-protein).

T cells derived from spleen and lung were stimulated with a pool of 15-mer peptides and quantified antigen-specific T cells using IFN-γ and IL-4 ELISPOT assays. IFN-γ and IL-4 ELISpot assay was performed using a Mouse IFN-γ ELISPOT Basic kit (ALP) and a Mouse IL-4 ELISPOT Basic Kit. For cell activation control, cultures were treated with 10 ng/ml phorbol 12-myristate 13-acetate PMA (Sigma, St. Louis, Mich., USA) and 1 μg/ml of ionomycin (Sigma, St. Louis, Mich., USA). Complete medium (RPMI supplemented with 10% FBS) was used as a negative control. Splenocytes and lung lymphocytes (3×10⁵ cells) were stimulated in vitro with a pool of peptides, consisting mainly of 15-mer sequences with 11 amino acids overlap, covering the complete sequence of the nucleocapsid phosphoprotein (“N”) of SARS-Coronavirus 2 at a concentration of 1.5 μg/ml/peptide (Miltenyi Biotec; 130-126-699, Germany) at 37° C. for 16-18 h in precoated ELISpot plates (MSIPS4W10 from Millipore) coated with AN18 IFN-γ (1 μg/ml, Mabtech #3321-3-250) and 11B11 IL-4 (1 μg/ml, Mabtech #3311-3-250) coating antibody. The next day, cells were washed off and plates were developed using biotinylated R4-6A2 anti-IFNγ (Mabtech #3321-6-250) and BVD6-24G2 anti-IL-4 (Mabtech #3311-6-250) detection antibody, respectively. Wells were washed and treated for 1 h at RT with 1:30,000 diluted Extravidin-ALP Antibody (Sigma, St. Louis, Mich., USA). After washing, spots were developed by adding 70 μL/well of BCIP/NBT-plus substrate (Mabtech #3650-10) to the wells. Plates were incubated for 20-30 minutes for color development and subsequently washed with water. Spots were quantified using Cytation 7 (Bio-Tek Instruments, Inc.). Each spot corresponds to an individual cytokine-secreting cell. Values are shown as the background-subtracted average of measured triplicates.

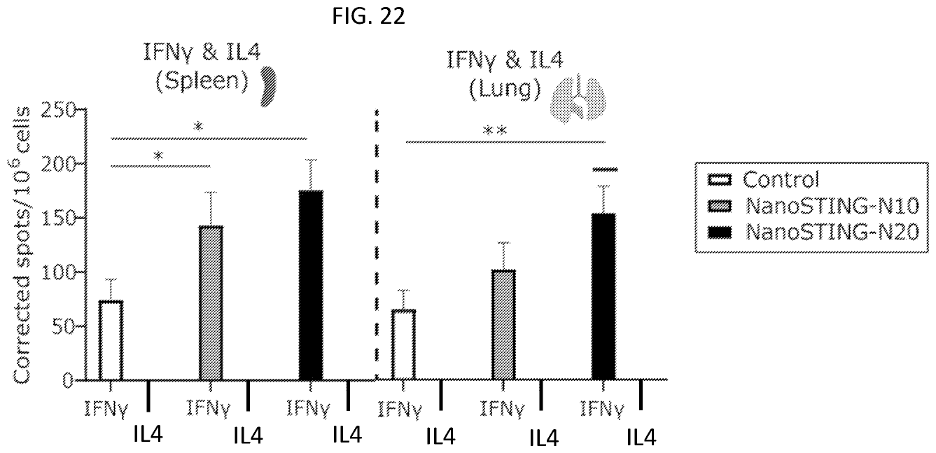

Nano-STING-N10 and NanoSTING-N20 immunized mice showed robust and significant splenic T cell responses with a mean of 143 and 176 IFN-γ spots/10⁶ cells, respectively (FIG. 22, gray and black bars to left of dotted line). Animals immunized with NanoSTING-N10 and NanoSTING-N20 showed elevated T cell responses in the lung with a mean of 102 and 154 IFN-γ spots/10⁶ respectively (FIG. 22, gray and black bars to right of dotted line). In contrast to the IFN-γ (Th1/Tc1) responses, no measurable IL-4 (Th2) responses were observed upon treatment with NanoSTING-N10 and NanoSTING-N20 for either splenic or lung cells (FIG. 22). Collectively, these results established that intranasal vaccination elicited strong Th1 responses with no evidence of Th2 responses.

### Example 14: Determination of Nucleocapsid-Specific Memory in T Cells Treated with NanoSTING-N Compositions

This example shows cell surface and intracellular cytokine staining of splenocytes and lung lymphocytes from subjects treated with NanoSTING-N compositions for subsequent flow cytometry analysis.

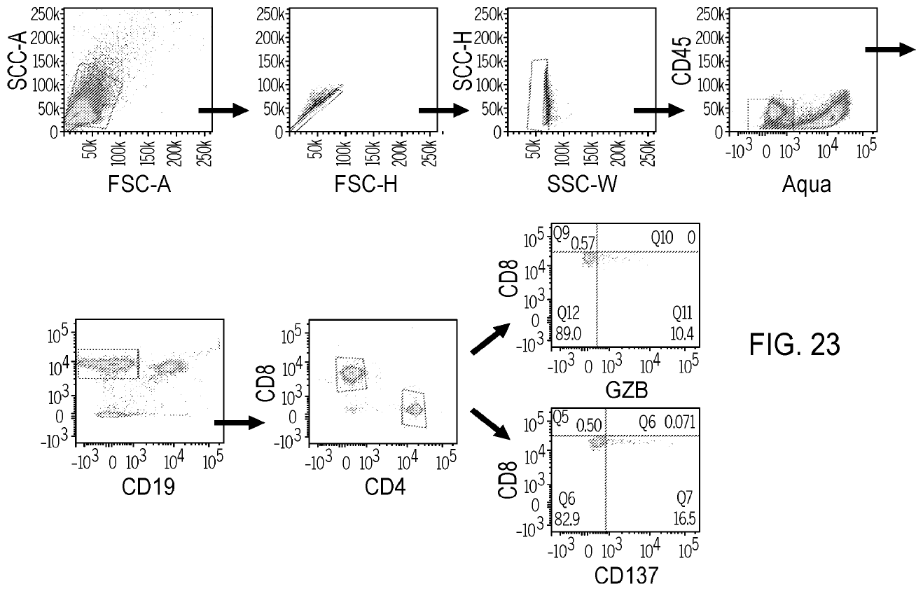

CD8+ T-cell responses can complement antibody mediated responses and can offer protection independent of antibody responses. Activation and function of N-protein specific memory CD8+ T cells in the lung airways, lung parenchyma, and spleen was investigated in these experiments. Spleen and lung lymphocytes from NanoSTING-N-treated and control-treated animals were stimulated with nucleocapsid protein-peptide pool at a concentration of 1.5 μg/ml/peptide (Miltenyi Biotec; 130-126-699, Germany) at 37° C. for 16-18 hours to determine nucleocapsid protein-specific CD8+ T cell responses at 18 hours after exposure to NanoSTING-N compositions. Brefeldin A (5 μg/ml BD Biosciences #BD 555029) was added for the last 5 hours of the incubation. 10 ng/ml PMA (Sigma, St. Louis, Mich., USA) and 1 μg/ml ionomycin (Sigma, St. Louis, Mich., USA) were used as a positive control. Stimulation without nucleocapsid peptides served as background control. Cells were collected and stained with Live/Dead Aqua (Thermo Fisher #L34965) in PBS, followed by Fc-receptor blockade with anti-CD16/CD32 (Thermo Fisher #14-0161-85), and then stained for 30 minutes on ice with the following antibodies in flow cytometry staining buffer (FACS): anti-CD4 AF589 (clone GK1.5; Biolegend #100446), anti-CD8b (clone YTS156.7.7; Biolegend #126609), anti-CD69 (clone H1.2F3; Biolegend #104537), anti-CD137 (clone 1AH2; BD; #740364), anti-CCR7 (clone 4B12; Biolegend #120124), anti-CD45 (clone 30-F11; BD; #564279). Cells were washed twice with the FACS buffer and then fixed with 100 μl IC fixation buffer (eBioscience) for 30 minutes at RT. Cells were permeabilized for 10 minutes with 200 μl permeabilization buffer (BD Cytofix solution kit). Intracellular staining was performed using the antibodies Alexa Fluor 488 interferon (IFN) gamma (clone XMG1.2; BD; #557735) and Granzyme B (clone GB11; Biolegend; #515407) overnight at 4° C. Next, cells were washed with FACS buffer and analyzed on LSR-Fortessa flow cytometer (BD Bioscience), using FlowJo™ software version 10.8 (Tree Star Inc, Ashland, Oreg., USA) and the flow cytometry gating strategy illustrated in FIG. 23. Results were calculated as the total number of cytokine-positive cells with background subtracted. Antibody concentration was optimized by titration.

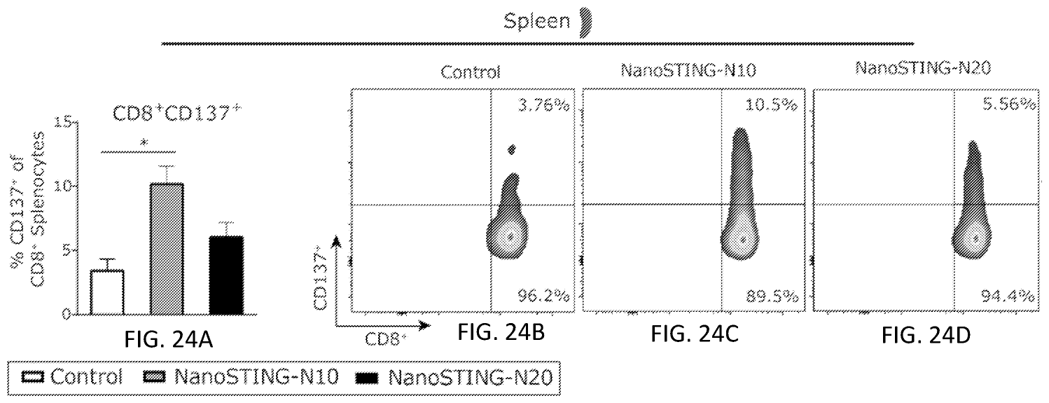

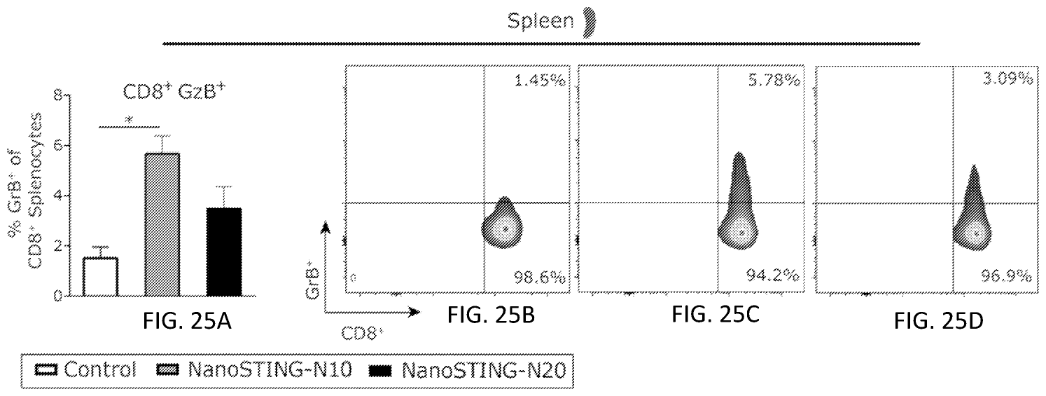

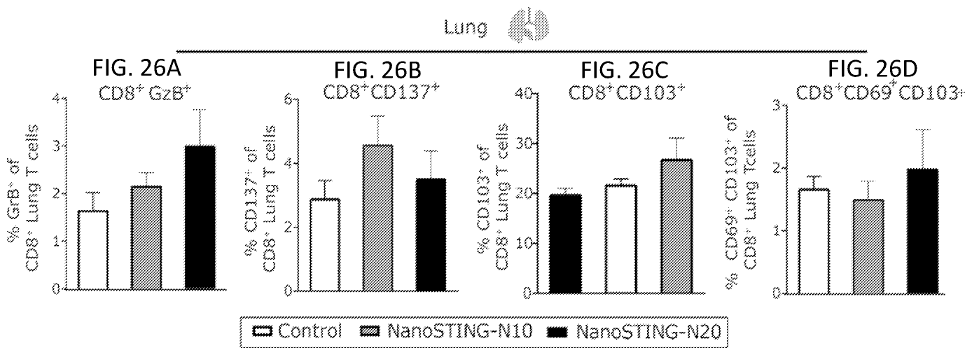

The effector molecule granzyme B (GzB) and the activation-induced marker CD137 were utilized to identify N-protein-reactive CD8+ T cells in the spleen and lungs. Restimulation ex vivo with a pool of N peptides as described above resulted in a large increase in the frequency of activated (CD8+CD137+) and cytotoxic (CD8+GzB+) T cells in the spleen, and to a lesser extent in the lung, of both the NanoSTING-N10 and NanoSTING-N20 vaccinated mice (FIG. 22). Percentages of CD137-positive CD8⁺ cells were increased in splenocytes treated with NanoSTING-N10, as compared to control treated-cells (see, FIG. 24A, FIG. 24B, FIG. 24C, and FIG. 24D). Percentages of GzB-positive CD8⁺ cells were also increased in splenocytes treated with NanoSTING-N10, as compared to control treated-cells (see, FIG. 25A, FIG. 25B, FIG. 25C, and FIG. 25D). The overall frequencies of the lung-resident CD103⁺CD69⁺CD8⁺ T cells was no different between the immunized animals and the control group (see, FIG. 26A, FIG. 26B, FIG. 26C, and FIG. 26D). Taken together, these results established that intranasal immunization cytotoxic T-cell responses in the lung and spleen.

### Example 15: Induction of Immune Response Using NanoSTING Compositions Comprising Chimeric Spike Proteins or RSV Antigen Proteins

This example illustrates comprehensive immunity conferred upon subjects treated with NanoSTING compositions comprising a chimeric spike protein antigen (NanoSTING-ChimS) and subjects treated with NanoSTING compositions comprising an RSV antigen (NanoSTING-RSV).

A recombinant form of the fusion glycoprotein from respiratory syncytial virus B1 containing a C-terminal histidine tag was produced by baculovirus infection of Trichoplusia in insect larvae and purified by chromatographic methods.

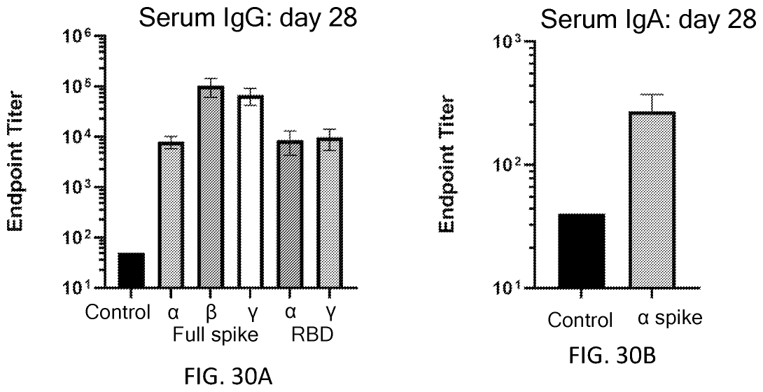

Serum was collected from subjects treated with NanoSTING compositions (NanoSTING-ChimS) comprising a liposome encapsulating cGAMP modulator and an alpha variant spike protein trimer, (2) a beta variant spike protein trimer, (3) a gamma variant spike protein trimer (“NanoSTING-varS”), (4) a receptor binding domain (RBD) portion of an alpha variant spike protein, or (5) an RBD portion of a gamma variant spike protein. Samples obtained from subjects at day 28 following treatment with compositions of each of groups (1) through (5) show increased antigen-specific IgG titers as compared to treatment with control group animals immunized with antigen but without adjuvant (FIG. 30A). FIG. 30B shows ELISA quantification of serum-derived IgA reactivity to the alpha variant of the chimeric spike protein trimer using serum samples obtained from subjects at day 28 following treatment with NanoSTING-ChimS compositions comprising a liposome, the alpha variant spike protein trimer antigen, and a STING agonist versus treatment with control compositions described herein comprising a liposome lacking the spike protein trimer antigen and the STING agonist. IgA titers are increased in samples from subjects treated with the NanoSTING-ChimS composition compared to samples from control-treated subjects.

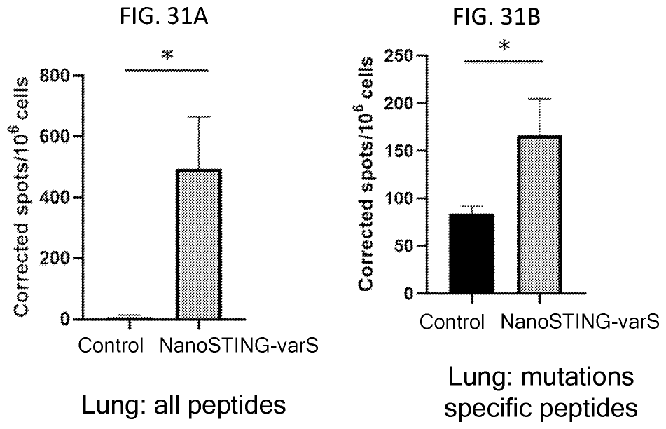

FIG. 31A shows interferon gamma (IFNγ) responses, as quantified using ELISpot assay, in lung samples of subjects treated with NanoSTING-ChimS compositions, each comprising a different spike protein (S-protein) variant of the peptide pool and together representing all of the S-protein-derived peptide pool versus treatment with a control composition comprising a liposome and lacking a chimeric spike protein. Interferon gamma levels were significantly increased in NanoSTING-treated animals' lung samples as compared to samples from the lungs of control-treated animals, showing the immunostimulatory effect of NanoSTING compositions is robust and broadly effective across all antigen variants. FIG. 31B shows interferon gamma responses, as quantified using ELISpot assay, in lung samples of subjects treated with NanoSTING-ChimS composition comprising a liposome, a gamma variant chimeric spike protein trimer antigen, and a STING agonist versus samples of subjects treated with a control composition comprising a liposome lacking a chimeric spike protein. Interferon gamma levels were significantly increased in NanoSTING-treated animals' lung samples versus samples from the lungs of control-treated animals, illustrating that the effect of NanoSTING compositions on subject immune response is also specific for individual antigen variants.

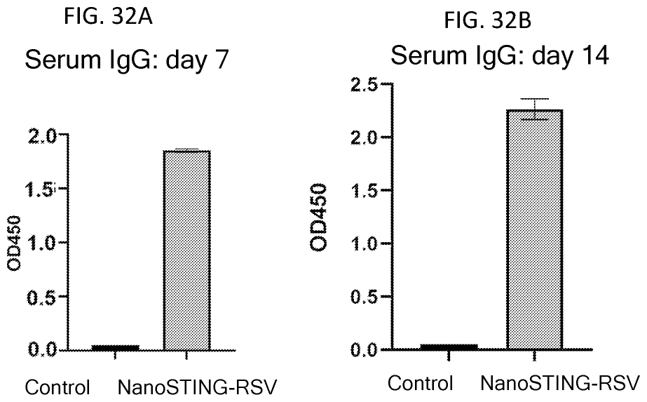

FIG. 32A and FIG. 32B show reactivity of IgG to B1 strain respiratory syncytial virus F protein (RSV-F) in ELISA assays performed using serum obtained from subjects at day 7 and day 14, respectively, following treatment with compositions described herein comprising a liposome, an RSV-F-derived antigen, and a STING agonist (“NanoSTING-RSV”) versus treatment with a control composition described herein comprising a liposome lacking a chimeric spike protein. RSV antigen-specific IgG levels are increased at both Day 7 and Day 14 compared to samples from control-treated animals, indicating comprehensive immunity conferred upon subjects after treatment with NanoSTING compositions comprising an antigen.

Unless otherwise defined, all technical terms used herein have the same meaning as commonly understood by one of ordinary skill in the art to which this subject matter belongs. It is to be understood that both the foregoing general description and the following detailed description are illustrative and explanatory, and are not restrictive of the subject matter, as claimed.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

As used herein, the singular forms “a,” “an,” and “the” are intended to include the plural forms as well, unless the context clearly indicates otherwise. For example, the word “a” or “an” means “at least one”, and the use of “or” means “and/or”, unless specifically stated otherwise. As used herein, the term “and/or” includes any and all combinations of one or more of the associated listed items. It will be further understood that the terms “comprises” and/or “comprising,” or “includes” and/or “including,” when used in this specification, specify the presence of stated features, regions, integers, steps, operations, elements and/or components, but do not preclude the presence of addition of one or more other features, regions, integers, steps, operations, elements, components, and/or groups thereof. Also, terms such as “element” or “component” encompass both elements or components comprising one unit and elements or components that comprise more than one unit unless specifically stated otherwise.

As used herein A and/or B encompasses one or more of A or B, and combinations thereof such as A and B. It will be understood that although the terms “first,” “second,” “third,” etc. may be used herein to describe various elements, components, regions and/or sections, these elements, components, regions and/or sections should not necessarily be limited by these terms. These terms may be used merely to distinguish one element, component, region or section from another element, component, region, or section. Thus, a first element, component, region or section discussed herein could be termed a second element, component, region or section without departing from the teachings of the present disclosure, in some cases.

The term “subject” as used herein includes mammals. Mammals include rats, mice, non-human primates, and primates, including humans.

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the invention, subject to any specifically excluded limit in the stated range. Where the stated range includes one or both of the limits, ranges excluding either or both of those included limits are also included in the invention, unless the context clearly dictates otherwise

As used in this specification and the claims, unless otherwise stated, the term “about,” and “approximately,” or “substantially” refers to variations of less than or equal to +/−0.1%, +/−1%, +/−2%, +/−3%, +/−4%, +/−5%, +/−6%, +/−7%, +/−8%, +/−9%, +/−10%, +/−11%, +/−12%, +/−14%, +/−15%, or +/−20%, including increments therein, of the numerical value depending on the embodiment. As a non-limiting example, about 100 nanometers (nm) represents a range of 95 nanometers to 105 nanometers (which is +/−5% of 100 nanometers), 90 to 110 nanometers (which is +/−10% of 100 nanometers), or 85 nanometers to 115 nanometers (which is +/−15% of 100 nanometers) depending on the embodiments.

Although certain embodiments and examples are provided in the foregoing description, the inventive subject matter extends beyond the specifically disclosed embodiments to other alternative embodiments and/or uses, and to modifications and equivalents thereof. In any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not necessarily be construed to imply that these operations are order dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A composition for use in treating or preventing a disease in an animal comprising:
a lipid-based particle comprising an antigen and a modulator,
wherein the modulator is a pattern recognition receptor agonist.

2. (canceled)

3. The composition of claim 1, wherein the lipid-based particle is a liposome.

4. The composition of claim 1, wherein the lipid-based particle comprises DPPG (1,2-dipalmitoyl-sn-glycero-3-phospho-(1′-rac-glycerol)), DPPE (1,2-bis(diphenylphosphino)ethane), DPPC (1,2-dipalmitoyl-sn-glycero-3-phosphocholine), DPPE-PEG2000 (1,2-dipalmitoyl-sn-glycero-3-phosphoethanolamine-N-[methoxy(polyethylene glycol)-2000]), cholesterol, or a combination thereof.

5. The composition of claim 1, wherein the lipid-based particle comprises DPPC, DPPG, cholesterol, and DPPE-PEG2000.

6. The composition of claim 5, wherein the lipid-based particle comprises the DPPC, the DPPG, the cholesterol, and the DPPE-PEG2000 in a 10:1:1:1 ratio, respectively.

7. The composition of claim 1, wherein the antigen is associated with an outer surface of the lipid-based particle.

8. The composition of claim 1, wherein at least a portion of the antigen is integrated into a membrane of the lipid-based particle.

9. The composition of claim 1, wherein at least a portion of the antigen is encapsulated within the lipid-based particle.

10. The composition of claim 1, wherein the antigen elicits an immune response against the disease.

11. The composition of claim 1, wherein the antigen comprises a spike protein or portion thereof.

12. The composition of claim 1, wherein the antigen comprises a nucleocapsid protein or portion thereof.

13. The composition of claim 1, wherein the modulator is capable of activating or inhibiting of the stimulator of interferon genes (STING) pathway in the subject.

14. The composition of claim 13, wherein the modulator is an agonist of the STING pathway.

15. (canceled)

16. (canceled)

17. (canceled)

18. (canceled)

19. (canceled)

20. (canceled)

21. (canceled)

22. (canceled)

23. The composition of claim 1, wherein the composition is lyophilized.

24. The composition of claim 1, wherein the composition is formulated in unit dose formulation as a monotherapy.

25. The composition of any one of claim 1, wherein the composition is suitable for developing immunity in the subject against the disease.

26. The composition of claim 1, wherein the composition elicits an innate immune response in the subject against the disease.

27. (canceled)

28. (canceled)

29. A method of treating or preventing a disease in a subject, said method comprising: administering to the subject a composition comprising a lipid-based particle, wherein the lipid-based particle comprises an antigen and a modulator, wherein the modulator is a pattern recognition receptor agonist.

30. (canceled)

31. The method of claim 29, wherein the composition is suitable for developing immunity in the subject against the disease.

32. The method of claim 29, wherein administering the composition to the subject elicits an immune response in the subject against the disease.

33. The method of claim 32, wherein administering the composition elicits an innate immune response in the subject against the disease.

34. The method of claim 33, wherein the innate immune response leads to the activation of interferon regulator factors (IRFs) in the subject.

35. The method of claim 33, wherein the innate immune response leads to the activation of nuclear factor κB (NF-κB).

36. The method of claim 33, wherein the innate immune response leads to the synthesis and secretion of type-I and type-III interferons and the subsequent upregulation of IFN-stimulated genes (ISGs).

37. The method of claim 33, wherein the innate immune response leads to associated adaptive immunity.

38. The method of claim 29, wherein the disease is caused by a pathogen.

39. The method of claim 38, wherein the pathogen is a respiratory pathogen.

40. The method of claim 39, wherein the respiratory pathogen is a virus.

41. The method of claim 40, wherein the virus is selected from a severe acute respiratory syndrome coronavirus (SARS-CoV), a severe acute respiratory syndrome-related coronavirus (SARSr-CoV), a human coronavirus 229E (HCoV-229E), a human coronavirus NL63 (HCoV-NL63), a human coronavirus OC43 (HCoV-OC43), a human coronavirus HKU1 (HCoV-HKU1), a Middle East respiratory syndrome-related coronavirus (MERS-CoV), a severe acute respiratory syndrome-related coronavirus 2 (SARS-CoV-2), a variant of SARS-CoV-2, or combinations thereof.

42. The method of claim 29, wherein the composition is administered to the subject by intravenous administration, intramuscular administration, intradermal administration, intraperitoneal administration, subcutaneous administration, spray-based administration, aerosol-based administration, in ovo administration, oral administration, intraocular administration, intratracheal administration, intranasal administration, inhalational administration, or combinations thereof.

43. The method of claim 29, wherein the composition is administered to the subject in single dose.

44. The method of claim 29, wherein the composition is administered to the subject in at least two doses, a first dose and a second dose of the at least two doses being administered at an interval of at least 6 hours, at least 12 hours, at least 24 hours, at least 36 hours, at least 2 days, at least 3 days, at least 4 days, at least 5 days, at least 6 days, at least 7 days, at least 14 days, or at least 28 days.

45. The method of claim 29, wherein the subject is a mammal.

46. The method of any one of claim 29, wherein the method is used to prevent the establishment of the disease in the subject, to prevent progression of the disease in the subject, to prevent the transmission of disease to a second subject, or combinations thereof.

47. (canceled)

48. (canceled)
